(19) United States
(12) Patent Application Publication
(10) Pub. No.: US 2013/0344607 A1
(43) Pub. Date: Dec. 26, 2013

(54) PI-CONJUGATED FLUOROIONOPHORES AND METHOD FOR DETERMINING AN ALKALI ION

(71) Applicants: Sandra Ast, Potsdam (DE); Hans-Juergen Holdt, Juelchendorf (DE)

(72) Inventors: Sandra Ast, Potsdam (DE); Hans-Juergen Holdt, Juelchendorf (DE)

(73) Assignee: UNIVERSITAET POTSDAM, Potsdam (DE)

(21) Appl. No.: 14/003,909

(22) PCT Filed: Mar. 9, 2012
(86) PCT No.: PCT/EP2012/054181

§ 371 Date: Sep. 9, 2013

(30) Foreign Application Priority Data
Mar. 18, 2011 (EP) 11158899.2

(51) Int. Cl. G01N 21/64; C07D 498/08; C07D 413/14

(52) U.S. Cl. CPC G01N 21/643; C07D 413/14; C07D 498/08
USPC 436/79; 540/467; 540/469

(57) ABSTRACT

The present invention relates to π-conjugated fluoroionophores, methods for their preparation, and their use and a method for determining an alkali ion.

Fluoroionophoric compounds of the general formula I are described

Ionophore-π-Linker-Fluorophore (I)

wherein
the Ionophore is an anilino containing crown ether or cryptand with one or more anilino donor moieties as electron donors, forming a stable complex with an alkali metal ion
the π-Linker is an aromatic or heteroaromatic conjugative linking moiety, and the Fluorophore is an electron acceptor moiety.

Variation of the ionophoric unit offers a broad spectrum of detectable K⁺ and Na⁺-concentrations, ranging from high concentration around 800 mM down to very low concentrations around 3 mM.

The fluoroionophores have great potential for application in fluorescent optode system based blood analyzing equipment for methods and kits for the determination of K⁺ and Na⁺ concentrations in biological systems, either in vitro or in vivo, using embodiments of the disclosed fluoroionophores.

The present invention relates to π-conjugated fluoroionophores, methods for their preparation, and their use and a method for determining an alkali ion.

### BACKGROUND OF THE INVENTION

EP 0 881 487 A2 discloses a method for the determination of an alkali ion in biological samples like blood. The determination is performed by using a compound having a luminophoric and an ionophoric moiety. WO 2007/0044866 A2 describes chromoionophoric compounds comprising a triazaeryptand (TAO) K⁺ ionophore conjugated to at least a first chromophoric moiety. These water-soluble fluorescent compounds are used for the detection of potassium. U.S. Pat. No. 6,211,359 B1 discloses a triaza-cryptand useful as a luminescence indicator for alkali ions. US 2007/0259443 A1 and US 2007/0259444 A1 disclose chromoionophores and a method of determining potassium ions.

Recently 1,2,3-triazol-1,4-diyl fluoroionophores for Zn²⁺,¹Ni²⁺,² Cu²⁺,³ Hg²⁺,⁴ Ag⁺,⁴ and Al³⁺,⁶ were generated by Cu(I) catalyzed reaction between an azide and an alkyne (CuAAC). In these fluoroionophores the 1,2,3-triazol-1,4-diyls serve in addition to the conventional function as covalent linkers as both, chelating ligand of the metal ions and as electronic transmitter of a coordination event to the fluorophore.^{1a} Only Zn²⁺ and Al³⁺ could be detected by fluorescent enhancement, whereas the other metal ions were analysed through fluorescence quenching. In these known 1,2,3-triazol-1,4-diyl fluoroionophores, no electronically conjugated signal transduction chain: ionophore-1,2,3-triazole-fluorophore, is used. Either receptor and 1,2,3-triazole are connected through a deconjugated linker^{1,2} or the triazole and the fluorophoric group are deconjugated.^{3,4,5}

In a systematic investigation Diederich et al. showed the capacity of the 1,2,3-triazol-diyls as active π-linkers in Charge Transfer (CT) chromophores.⁶ Such push-pull chromophores are only weak or even non fluorescent.

The compounds and methods known show several disadvantages when used for the determination of potassium especially in biological samples like blood. A disadvantage of the known methods is that the cations can only be measured within a small concentration range. Further these methods also lack sensitivity and selectivity especially in the presence of sodium.

Therefore, it is an object of the present invention to provide a method for the determination of alkali metal cations in biological samples within a broad concentration range, and with improved sensitivity and specificity.

### BRIEF DESCRIPTION OF THE INVENTION

The inventors found that in simple CuAAC-generated systems the electronic conjugation of an alkali metal ion selective receptor which could be a N-phenylaza-crown ether or a N-phenylaza-cryptand, and a fluorophore through a 1,2,3-triazol π-linker results in a perfect signal transduction chain for a simultaneous fluorescence quenching via both, an internal charge transfer (ICT) and a photoinduced electron transfer (PET). The fluorescence of the original fluorophore is basically nullified. Coordination of an alkali metal ion to the anilino-donor of the ionophoric unit interrupts this π-conjugation as well as the PET, resulting in a revival of the fluorescence, proportional to the metal ion concentration. This concentration dependant fluorescence enhancement allows the sensing of K⁺ and Na⁺ under simulated physiological conditions.

The problem of the invention is solved by the development of novel triazole-based fluoroionophores which show significant selectivity for Na⁺ and K⁺ in water though bearing a simple crown-ether-receptor or cryptand-receptor unit.

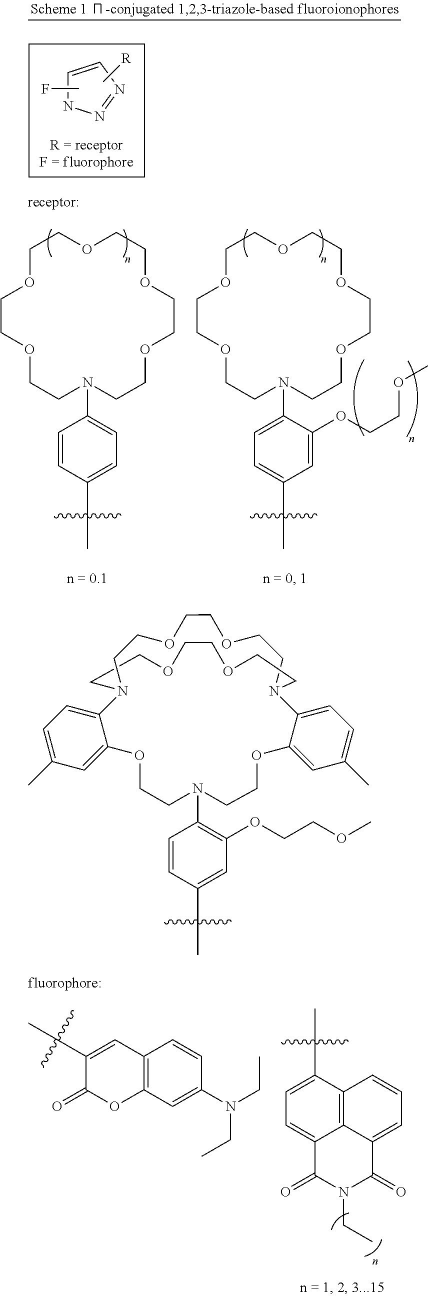

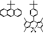

To the best of the inventors knowledge no 1,2,3-triazole-based linker between receptor and fluorophore, giving a conjugated system exists to now.

Variation of the ionophoric unit offers a broad spectrum of detectable K⁺ and Na⁺-concentrations, ranging from high concentration around 800 mM down to very low concentrations around 3 mM.

The new alkali metal ion 1,2,3-triazole-based fluoroionophores have great potential for application in fluorescent optode systems based blood analyzing equipment for methods and kits for the determination of K⁺ and Na⁺ concentrations in biological systems, either in vitro or in vivo, using embodiments of the fluoroionophores according to the present invention.

The problem is solved according to the invention by providing a fluoroionophoric compound of the general formula I

Ionophore-π-Linker-Fluorophore (I)

wherein
the Ionophore is an anilino containing crown ether or cryptand with one or more anilino donor moieties as electron donors, forming a stable complex with an alkali metal ion
the π-Linker is an aromatic or heteroaromatic conjugative linking moiety, and the Fluorophore is an electron acceptor moiety.

Preferred are compounds according to the invention, wherein the ionophore is selected from the group consisting of

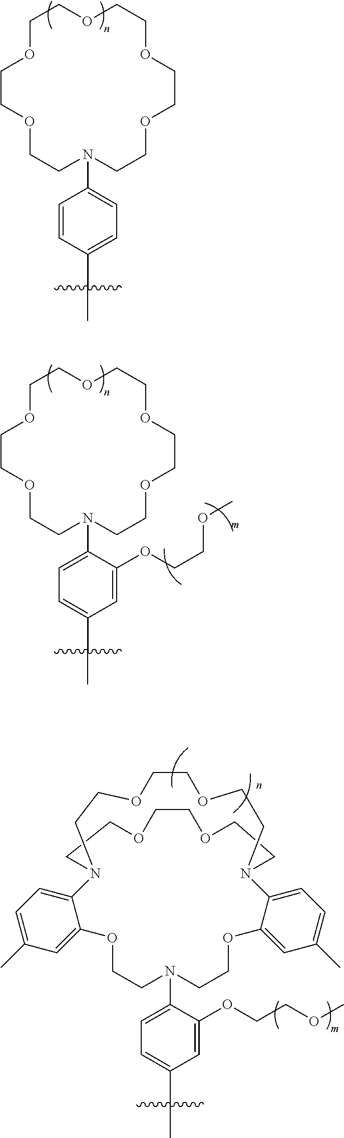

wherein
n is a number selected from 0 and 1,
m is a number selected from 0, 1, and 2, and
wherein the phenyl ring is optionally substituted with halogen, nitro, amino, hydroxyl, lower alkyl or lower alkoxy, wherein the lower alkyl or lower alkoxy are optionally substituted with halogen, nitro, amino, hydroxyl or lower alkyl or lower alkoxy, and
wherein the phenyl ring may optionally be a part of a condensed aromatic system, that is optionally substituted with halogen, nitro, amino, hydroxyl, or lower alkyl or lower alkoxy or phenyl, optionally substituted with halogen, nitro, amino, hydroxyl or lower alkyl or lower alkoxy.

Preferred are also compounds according to the invention, wherein the π-Linker is selected from the group consisting of an aromatic or heteroaromatic moiety, wherein the aromatic moiety refers to a 6- to 14-membered monocyclic, bicyclic or tricyclic aromatic hydrocarbon ring system that is unsubstituted or optionally substituted with one or more substituents, and wherein the heteroaromatic moiety refers to an aromatic heterocyclic ring of 5 to 14 members and having at least one heteroatom selected from nitrogen, oxygen and sulfur, and containing at least 1 carbon atom, including monocyclic, bicyclic, and tricyclic ring systems, that is unsubstituted or optionally substituted with one or more substituents.

Especially preferred are compounds, wherein the π-Linker is selected from the group consisting of phenyl and naphthyl, that are unsubstituted or optionally substituted with one or more substituents, and triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, pyrimidyl, azepinyl, oxepinyl, naphthothiazolyl, quinoxalinyl, that are unsubstituted or optionally substituted with one or more substituents.

Especially preferred are compounds according to the invention, wherein the π-Linker is selected from the group consisting of isomeric disubstituted 1,2,3-triazoles, preferably:

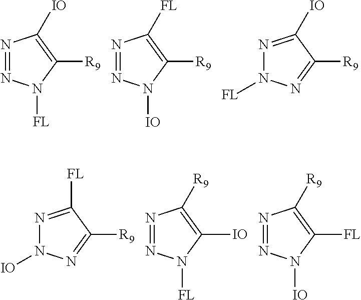

wherein
IO is the ionophore according to claim 1;
Fl is the fluorophore according to claim 1;
R₉ is selected from hydrogen, halogen, nitro, amino, hydroxyl, lower alkyl and lower alkoxy, optionally substituted with halogen, nitro, amino, hydroxyl or lower alkyl or lower alkoxy.

Very specially preferred are compounds according to the invention, wherein the π-Linker is selected from the group consisting of substituted 1,4-triazoles, namely

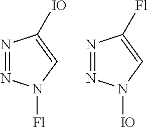

wherein IO is the ionophore and Fl is the fluorophore.

Preferred are compounds according to the invention, wherein the fluorophore moiety is represented by the formula

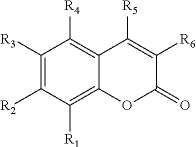

wherein

R¹, R⁴, R⁵═H, lower alkyl, CF₃, MeO, halogen, NO₂, CN, R₂, R₃═H, NH₂, N(lower alkyl)₂, lower alkyl, optionally substituted with carboxyl or carbonyl, diethyl amino

R⁶=alkinyl, azide.

Preferred are also compounds according to the invention, wherein the fluorophore moiety is selected from the group consisting of

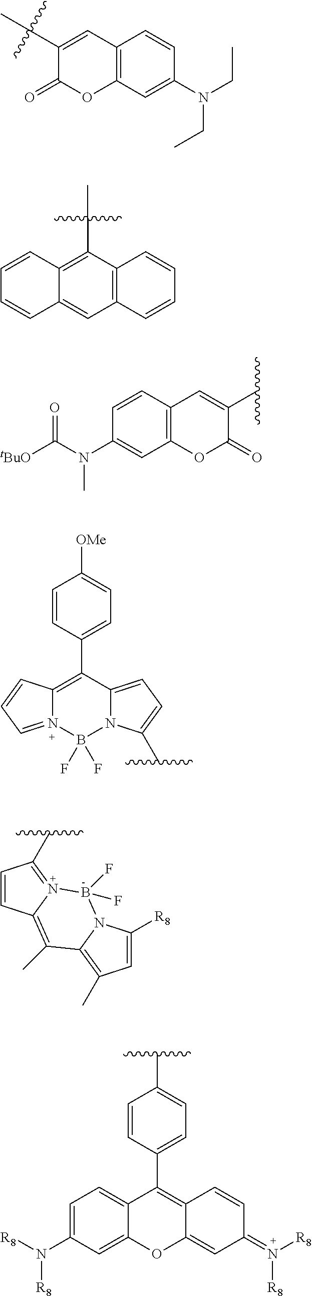

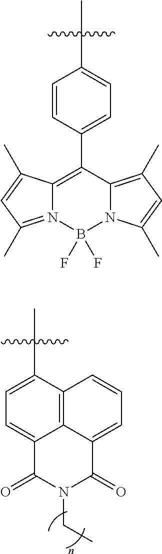

wherein
n is an integer ranging from 0 to 15;
R₈ is selected from hydrogen, lower alkyl or lower alkoxy;
and which are optionally substituted with halogen, nitro, amino, hydroxyl, lower alkyl or lower alkoxy.

Especially preferred are the following compounds of the invention according to general Formula (I), namely

### Coumarin Fluoroionophores

### Potassium Selective

### 3-{4-[4-(1,4,7,10,13-pentaoxa-16-azacyclooctadecan-16-yl)phenyl]-1H-1,2,3-triazol-1-yl}-7-(diethylamino)-2H-chromen-2-one

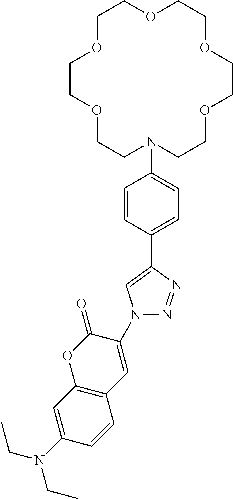 [SMILES: CCN(CC)c1ccc2cc(-n3cc(-c4ccc(N5CCOCCOCCOCCOCCOCC5)cc4)nn3)c(=O)oc2c1]

### 3-{1-[4-(1,4,7,10,13-pentaoxa-16-azacyclooctadecan-16-yl)phenyl]-1H-1,2,3-triazol-4-yl}-7-(diethylamino)-2H-chromen-2-one

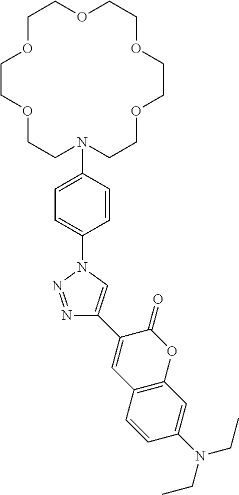 [SMILES: CCN(CC)c1ccc2cc(-c3cn(-c4ccc(N5CCOCCOCCOCCOCCOCC5)cc4)nn3)c(=O)oc2c1]

### 7-(diethylamino)-3-{1-[3-(2-methoxyethoxy)-4-(1,4,7,10,13-pentaoxa-16-azacyclooctadecan-16-yl)phenyl]-1H-1,2,3-triazol-4-yl}-2H-chromen-2-one

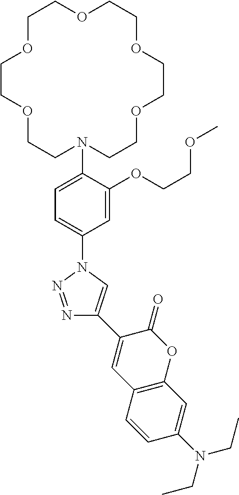 [SMILES: CCN(CC)c1ccc2cc(-c3cn(-c4ccc(N5CCOCCOCCOCCOCCOCC5)c(OCCOC)c4)nn3)c(=O)oc2c1]

### 7-(diethylamino)-3-{4-[3-(2-methoxyethoxy)-4-(1,4,7,10,13-pentaoxa-16-azacyclooctadecan-16-yl)phenyl]-1H-1,2,3-triazol-1-yl}-2H-chromen-2-one

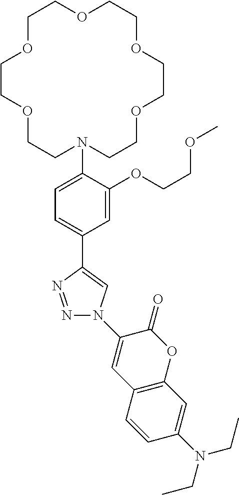 [SMILES: CCN(CC)c1ccc2cc(-n3cc(-c4ccc(N5CCOCCOCCOCCOCCOCC5)c(OCCOC)c4)nn3)c(=O)oc2c1]

### 7-(diethylamino)-3-{4-3-[2-methoxyethoxy)-4{bis(4-methylbenzo[5,6,17, 18](O,N,N′,O′)-]-1,7,16, -triaza-cryptand-[3,2,1]-phen-4-yl]-1H-1.2.3-triazol-1yl}-2H-chromen-2-one

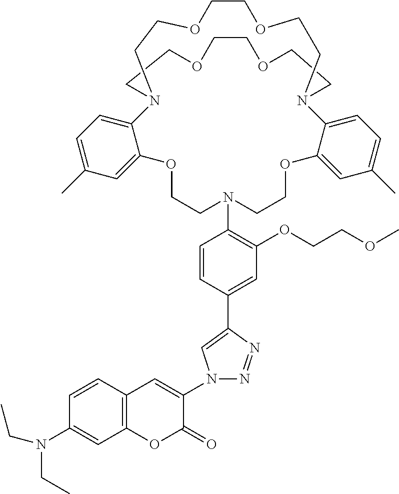 [SMILES: CCN(CC)c1ccc2cc(-n3cc(-c4ccc(N5CCOc6cc(C)ccc6N6CCOCCOCCN(CCOCCOCC6)c6ccc(C)cc6OCC5)c(OCCOC)c4)nn3)c(=O)oc2c1]

### 7-(diethylamino)-3-{4-[3-(2-methoxyethoxy]-4{bis(4-methylbenzo[5,6,17, 18](O,N,N′,O′)-]-1,7,16, -triaza-cryptand-[3,2,1]-phen-4-yl]-1H-1.2.3-triazol-1yl}-2H-chromen-2-one

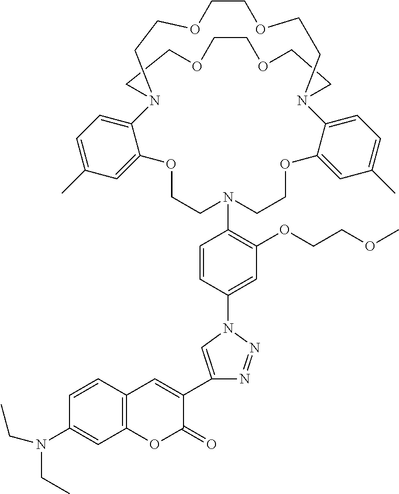 [SMILES: CCN(CC)c1ccc2cc(-c3cn(-c4ccc(N5CCOc6cc(C)ccc6N6CCOCCOCCN(CCOCCOCC6)c6ccc(C)cc6OCC5)c(OCCOC)c4)nn3)c(=O)oc2c1]

### Sodium Selective

### 3-{4-[4-(1,4,7,10-tetraoxa-13-azacyclopentadecan-13-yl)phenyl]-1H-1,2,3-triazol-1-yl}-7-(diethylamino)-2H-chromen-2-one

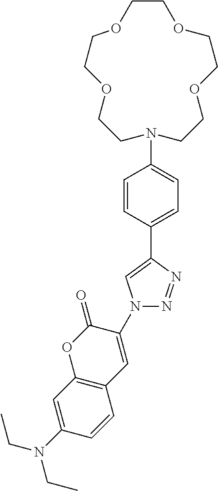 [SMILES: CCN(CC)c1ccc2cc(-n3cc(-c4ccc(N5CCOCCOCCOCCOCC5)cc4)nn3)c(=O)oc2c1]

### 3-{1-[4-(1,4,7,10-tetraoxa-13-azacyclopentadecan-13-yl)phenyl]-1H-1,2,3-triazol-4-yl}-7-(diethylamino)-2H-chromen-2-one

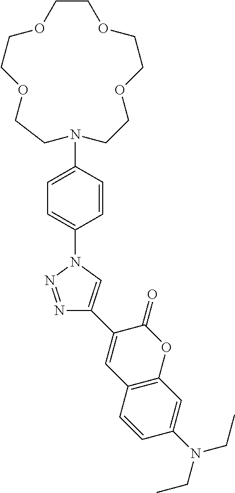 [SMILES: CCN(CC)c1ccc2cc(-c3cn(-c4ccc(N5CCOCCOCCOCCOCC5)cc4)nn3)c(=O)oc2c1]

### 7-(diethylamino)-3-{4-[3-methoxy-4-(1,4,7,10-tetraoxa-13-azacyclopentadecan-13-yl)phenyl]-1H-1,2,3-triazol-1-yl}-2H-chromen-2-one

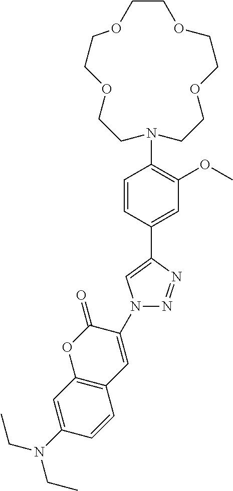 [SMILES: CCN(CC)c1ccc2cc(-n3cc(-c4ccc(N5CCOCCOCCOCCOCC5)c(OC)c4)nn3)c(=O)oc2c1]

### 7-(diethylamino)-3-{1-[3-methoxy-4-(1,4,7,10-tetraoxa-13-azacyclopentadecan-13-yl)phenyl]-1H-1,2,3-triazol-4-yl}-2H-chromen-2-one

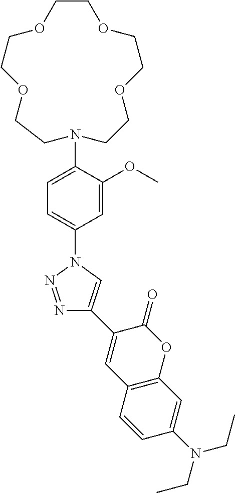 [SMILES: CCN(CC)c1ccc2cc(-c3cn(-c4ccc(N5CCOCCOCCOCCOCC5)c(OC)c4)nn3)c(=O)oc2c1]

### 7-(diethylamino)-3-{4-[3-methoxy-4{bis(4-methylbenzo[5,6,17,18](O,N,N′, O′)-]-1,7,16, -triaza-cryptand-[3,2,1]-phen-4-yl]-1H-1.2.3-triazol-1yl}-2H-chromen-2-one

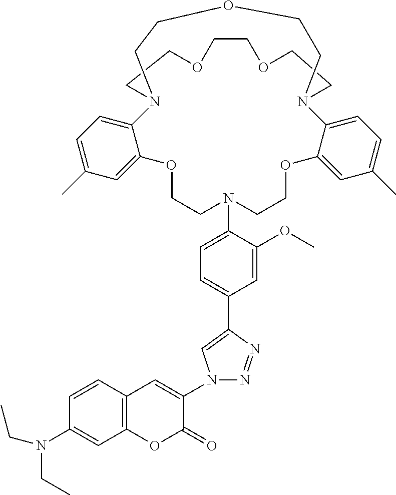 [SMILES: CCN(CC)c1ccc2cc(-n3cc(-c4ccc(N5CCOc6cc(C)ccc6N6CCOCCOCCN(CCOCC6)c6ccc(C)cc6OCC5)c(OC)c4)nn3)c(=O)oc2c1]

### 7-(diethylamino)-3-{1-[3-methoxy-4{bis(4-methylbenzo[5,6,17,18](O,N,N′, O′)-]-1,7,16, -triaza-cryptand-[3,2,1]-phen-1-yl]-1H-1.2.3-triazol-1yl}-2H-chromen-2-one

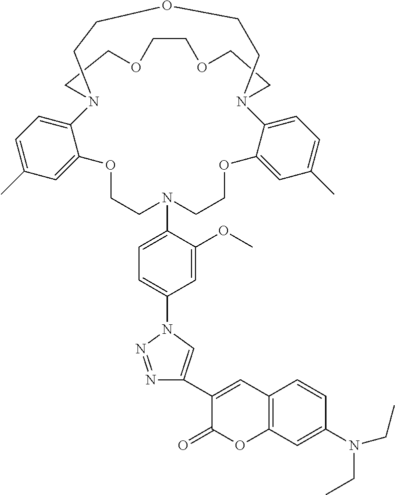 [SMILES: CCN(CC)c1ccc2cc(-c3cn(-c4ccc(N5CCOc6cc(C)ccc6N6CCOCCOCCN(CCOCC6)c6ccc(C)cc6OCC5)c(OC)c4)nn3)c(=O)oc2c1]

### Naphthalimid Fluoroionophores

### Potassium Selective

### 6-{4-[4-(1,4,7,10,13-pentaoxa-16-azacyclooctadecan-16-yl)phenyl]-1H-1,2,3-triazol-1-yl}-2-ethyl-1H-benzo[de]isoquinoline-1,3(2H)-dione

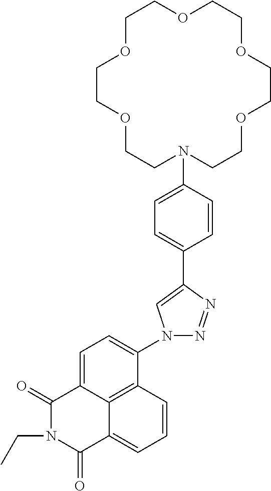 [SMILES: CCN1C(=O)c2cccc3c(-n4cc(-c5ccc(N6CCOCCOCCOCCOCCOCC6)cc5)nn4)ccc(c23)C1=O]

### 6-{1-[4-(1,4,7,10,13-pentaoxa-16-azacyclooctadecan-16-yl)phenyl]-1H-1,2,3-triazol-4-yl}-2-ethyl-1H-benzo[de]isoquinoline-1,3(2H)-dione

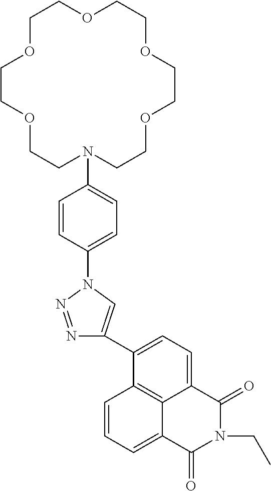 [SMILES: CCN1C(=O)c2cccc3c(-c4cn(-c5ccc(N6CCOCCOCCOCCOCCOCC6)cc5)nn4)ccc(c23)C1=O]

### 2-ethyl-6-{4-[3-(2-methoxyethoxy)-4-(1,4,7,10,13-pentaoxa-16-azacyclooctadecan-16-yl)phenyl]-1H-1,2,3-triazol-1-yl}-1H-benzo[de]isoquinoline-1,3(2H)-dione

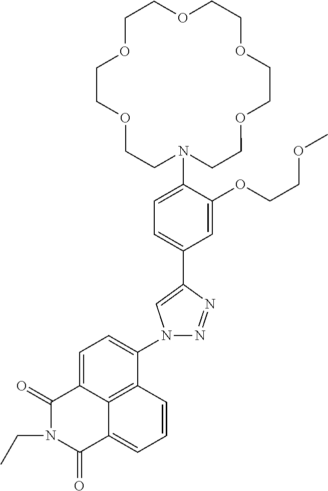 [SMILES: CCN1C(=O)c2cccc3c(-n4cc(-c5ccc(N6CCOCCOCCOCCOCCOCC6)c(OCCOC)c5)nn4)ccc(c23)C1=O]

### 2-ethyl-6-{1-[3-(2-methoxyethoxy)-4-(1,4,7,10,13-pentaoxa-16-azacyclooctadecan-16-yl)phenyl]-1H-1,2,3-triazol-4-yl}-1H-benzo[de]isoquinoline-1,3(2H)-dione

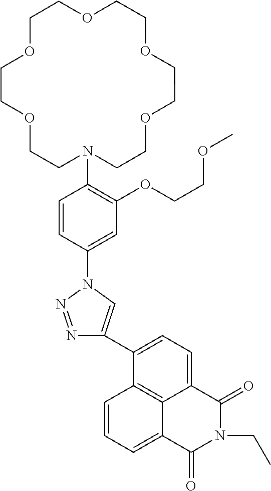 [SMILES: CCN1C(=O)c2cccc3c(-c4cn(-c5ccc(N6CCOCCOCCOCCOCCOCC6)c(OCCOC)c5)nn4)ccc(c23)C1=O]

### 2-ethyl-6-{4-[3-(2-methoxyethoxy)-4{bis(4-methylbenzo[5,6,17,18](O,N,N′, O′)-]-1,7,16, -triaza-cryptand-[3,2,2]-phen-1-yl]-1H-1.2.3-triazol 1-yl}-1H-benzo[de]isoquinoline-1,3(2H)-dione

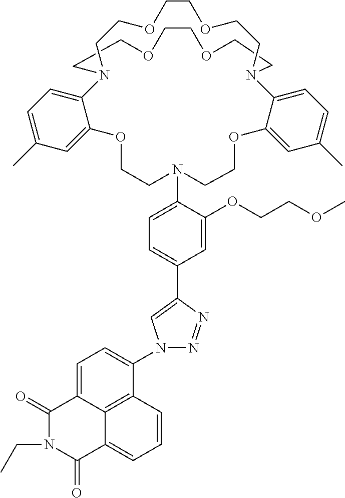 [SMILES: CCN1C(=O)c2cccc3c(-n4cc(-c5ccc(N6CCOc7cc(C)ccc7N7CCOCCOCCN(CCOCCOCC7)c7ccc(C)cc7OCC6)c(OCCOC)c5)nn4)ccc(c23)C1=O]

### 2-ethyl-6-{1-[3-(2-methoxyethoxy)-4{bis(4-methylbenzo[5,6,17,18](O,N,N′, O′)-]-1,7,16, -triaza-cryptand-[3,2,2]-phen-4-yl]-1H-1.2.3-triazol 1-yl}-1H-benzo[de]isoquinoline-1,3(2H)-dione

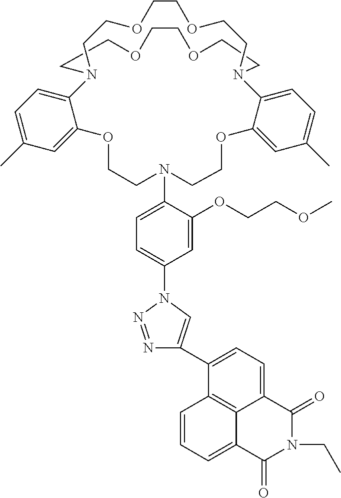 [SMILES: CCN1C(=O)c2cccc3c(-c4cn(-c5ccc(N6CCOc7cc(C)ccc7N7CCOCCOCCN(CCOCCOCC7)c7ccc(C)cc7OCC6)c(OCCOC)c5)nn4)ccc(c23)C1=O]

### Sodium Selective

### 6-{4-[4-(1,4,7,10-tetraoxa-13-azacyclopentadecan-13-yl)phenyl]-1H-1,2,3-triazol-1-yl}-2-ethyl-1H-benzo[de]isoquinoline-1,3(2H)-dione

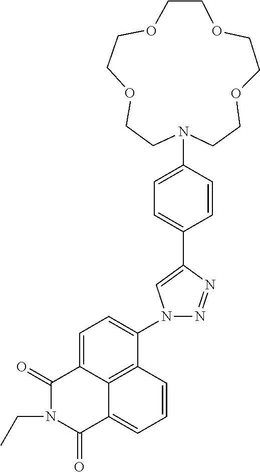 [SMILES: CCN1C(=O)c2cccc3c(-n4cc(-c5ccc(N6CCOCCOCCOCCOCC6)cc5)nn4)ccc(c23)C1=O]

### 6-{1-[4-(1,4,7,10-tetraoxa-13-azacyclopentadecan-13-yl)phenyl]-1H-1,2,3-triazol-4-yl}-2-ethyl-1H-benzo[de]isoquinoline-1,3(2H)-dione

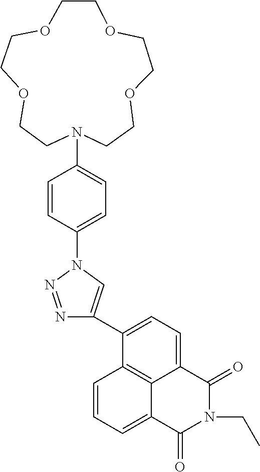 [SMILES: CCN1C(=O)c2cccc3c(-c4cn(-c5ccc(N6CCOCCOCCOCCOCC6)cc5)nn4)ccc(c23)C1=O]

### 2-ethyl-6-{4-[3-methoxy-4-(1,4,7,10-tetraoxa-13-azacyclopentadecan-13-yl)phenyl]-1H-1,2,3-triazol-1-yl}-1H-benzo[de]isoquinoline-1,3(2H)-dione

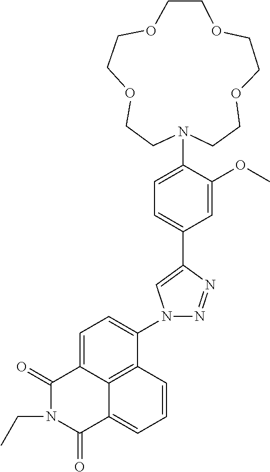 [SMILES: CCN1C(=O)c2cccc3c(-n4cc(-c5ccc(N6CCOCCOCCOCCOCC6)c(OC)c5)nn4)ccc(c23)C1=O]

### 2-ethyl-6-{1-[3-methoxy-4-(1,4,7,10-tetraoxa-13-azacyclopentadecan-13-yl)phenyl]-1H-1,2,3-triazol-4-yl}-1H-benzo[de]isoquinoline-1,3(2H)-dione

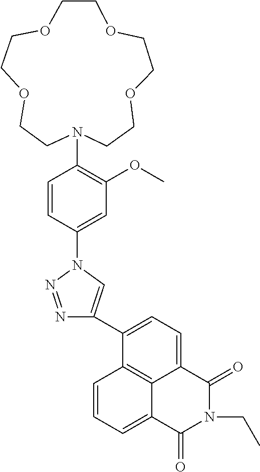 [SMILES: CCN1C(=O)c2cccc3c(-c4cn(-c5ccc(N6CCOCCOCCOCCOCC6)c(OC)c5)nn4)ccc(c23)C1=O]

### 2-ethyl-6-{4-[3-methoxy-4{bis(4-methylbenzo[5,6,17,18](O,N,N′,O′)-]-1,7, 16, -triaza-cryptand-[3,2,1]-phen-1-yl]-1H-1.2.3-triazol 1-yl}-1H-benzo[de]isoquinoline-1,3(2H)-dione

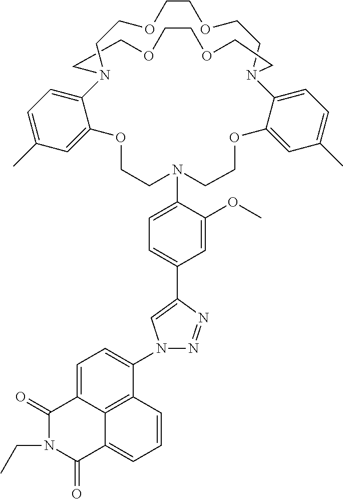 [SMILES: CCN1C(=O)c2cccc3c(-n4cc(-c5ccc(N6CCOc7cc(C)ccc7N7CCOCCOCCN(CCOCCOCC7)c7ccc(C)cc7OCC6)c(OC)c5)nn4)ccc(c23)C1=O]

### 2-ethyl-6-{1-[3-methoxy-4{bis(4-methylbenzo[5,6,17,18](O,N,N′,O′)-]-1,7, 16, -triaza-cryptand-[3,2,1]-phen-4-yl]-1H-1.2.3-triazol 1-yl}-1H-benzo[de]isoquinoline-1,3(2H)-dione

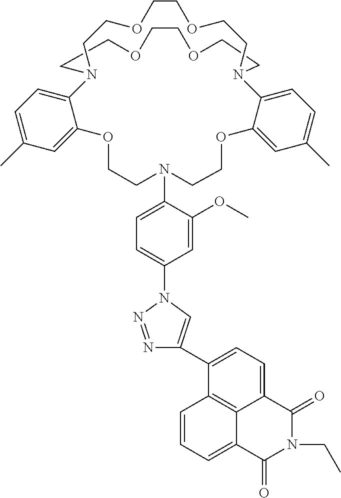 [SMILES: CCN1C(=O)c2cccc3c(-c4cn(-c5ccc(N6CCOc7cc(C)ccc7N7CCOCCOCCN(CCOCCOCC7)c7ccc(C)cc7OCC6)c(OC)c5)nn4)ccc(c23)C1=O]

### 7-BODIPY Fluoroionophores

### Potassium Selective

### 7-{4-[4-(1,4,7,10,13-pentaoxa-16-azacyclooctadecan-16-yl)phenyl]-1H-1,2,3-triazol-1-yl}-5,5-difluoro-1,3,9,10-tetramethyl-5H-dipyrrolo[1,2-c:1′,2′-f][1,3,2]diazaborinin-4-ium-5-uide

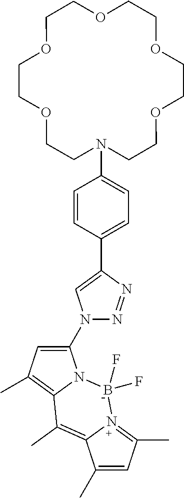

### 7-{1-[4-(1,4,7,10,13-pentaoxa-16-azacyclooctadecan-16-yl)phenyl]-1H-1,2,3-triazol-4-yl}-5,5-difluoro-1,3,9,10-tetramethyl-5H-dipyrrolo[1,2-c:1′,2′-f][1,3,2]diazaborinin-4-ium-5-uide

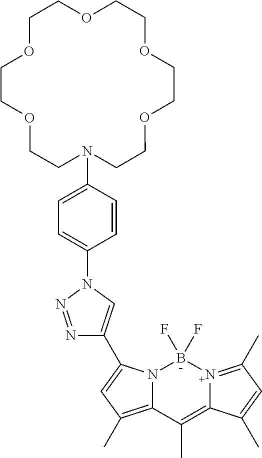

### 5,5-difluoro-7-{4-[3-(2-methoxyethoxy)-4-(1,4,7,10,13-pentaoxa-16-azacyclooctadecan-16-yl)phenyl]-1H-1,2,3-triazol-1-yl}-1,3,9,10-tetramethyl-5H-dipyrrolo[1,2-c:1′,2′f][1,3,2]diazaborinin-4-ium-5-uide

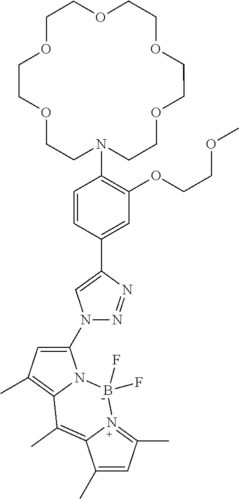

### 5,5-difluoro-7-{1-[3-(2-methoxyethoxy)-4-(1,4,7,10,13-pentaoxa-16-azacyclooctadecan-16-yl)phenyl]-1H-1,2,3-triazol-4-yl≡-1,3,9,10-tetramethyl-5H-dinvrrolo[1,2-c:1′,2′-f][1,3,2]diazaborinin-4-ium-5-uide

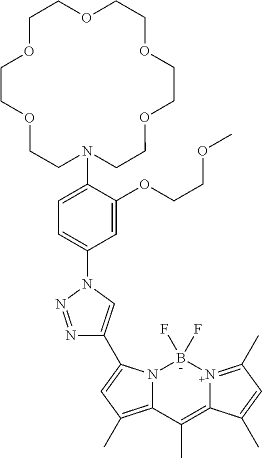

### 5,5-difluoro-7 {4-[3-(2-methoxyethoxy]-4(bis(4-methylbenzo[5,6,17,18](O, N,N′,O′)-]-1,7,16, -triaza-cryptand-[3,2,2]-phen-4-yl]-1H-1.2.3-triazol-1-yl}-4-1,3,9,10-tetramethyl-5H-dipyrrolo[1,2-c:1′,2′-f][1,3,2]diazaborinin-4-ium-5-uide

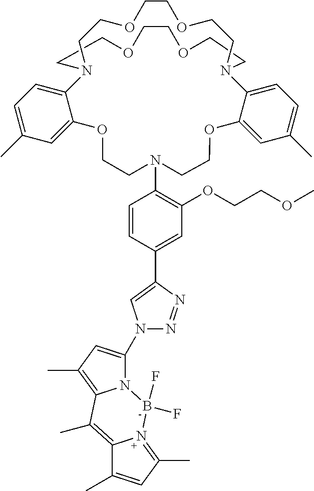

### 5,5-difluoro-7 {1-[3-(2-methoxyethoxy]-4(bis(4-methylbenzo[5,6,17,18](O, N,N′,O′)-]-1,7,16, -triaza-cryptand-[3,2,2]-phen-1-yl]-1H-1.2.3-triazol-1-yl}-4-1,3,9,10-tetramethyl-5H-dipyrrolo[1,2-c:1′,2′-f][1,3,2]diazaborinin-4-ium-5-uide

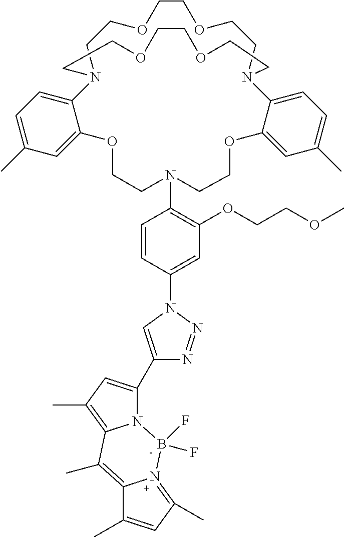

### Sodium Selective

### 7-{4-[4-(1,4,7,10-tetraoxa-13-azacyclopentadecan-13-yl)phenyl]-1H-1,2,3-triazol-1-yl}-5,5-difluoro-1,3,9,10-tetramethyl-5H-dipyrrolo[1,2-c:1′,2′-f][1,3,2]diazaborinin-4-ium-5-uide

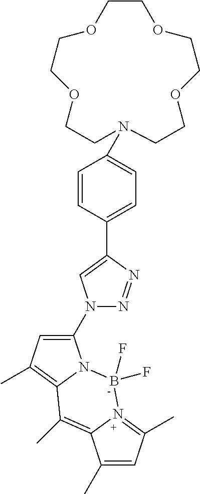

### 7-{1-[4-(1,4,7,10-tetraoxa-13-azacyclopentadecan-13-yl)phenyl]-1H-1,2,3-triazol-4-yl}-5,5-difluoro-1,3,9,10-tetramethyl-5H-dipyrrolo[1,2-c:1′,2′-f][1,3,2]diazaborinin-4-ium-5-uide

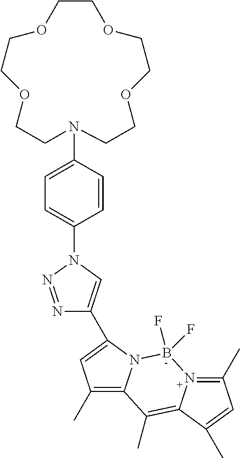

### 5,5-difluoro-7-{4-13-methoxy-4-(1,4,7,10-tetraoxa-13-azacyclopentadecan-13-yl)phenyl]-1H-1,2,3-triazol-1-yl}-1,3,9,10-tetramethyl-5H-dipyrrolo[1,2-c:1′,2′-f][1,3,2]diazaborinin-4-ium-5-uide

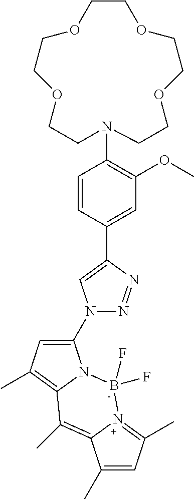

### 5,5-difluoro-7-{1-[3-methoxy-4-(1,4,7,10-tetraoxa-13-azacyclopentadecan-13-yl)phenyl]-1H-1,2,3-triazol-4-yl}-1,3,9,10-tetramethyl-5H-dipyrrolo[1,2-c:1′,2′-f][1,3,2]diazaborinin-4-ium-5-uide

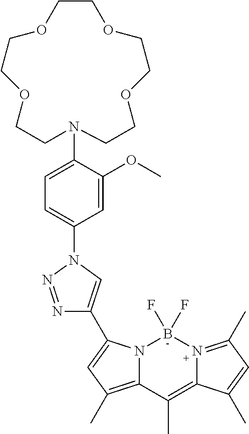

### 5,5-difluoro-7 {4-[3-methoxy-4(bis(4-methylbenzo[5,6,17,18](O,N,N′,O′)-]-1,7,16, -triaza-cryptand-[3,2,1]-phen-4-yl]-1H-1.2.3-triazol-1-yl}-4-1,3,9,10-tetramethyl-5H-dipyrrolo[1,2-c:1′,2′-f][1,3,2]diazaborinin-4-ium-5-uide

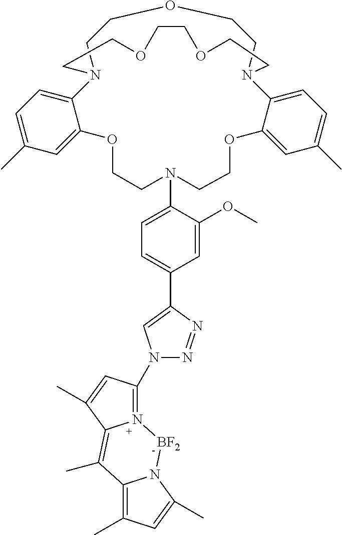

### 5,5-difluoro-7 {1-[3-methoxy-4(bis(4-methylbenzo[5,6,17,18](O,N,N′,O′)-]-1,7,16, -triaza-cryptand-[3,2,2]-phen-1-yl}-1H-1.2.3-triazol-1-yl}-4-1,3,9,10-tetramethyl-5H-dipyrrolo[1,2-c:1′,2′-f][1,3,2]diazaborinin-4-ium-5-uide

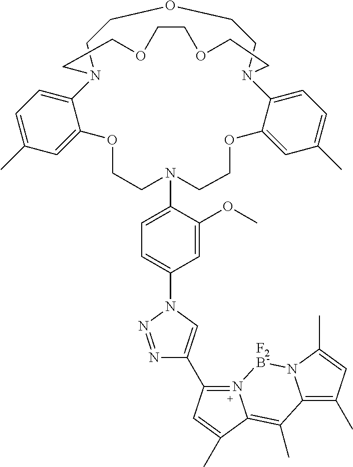

10-phenyl-BODIPY-fluoroionophores

### Potassium Selective

### 10-(4-{4-[4-(1,4,7,10,13-pentaoxa-16-azacyclooctadecan-16-yl)phenyl]-1H-1,2,3-triazol-1-yl}phenyl)-5,5-difluoro-1,3,7,9-tetramethyl-5H-dipyrrolo[1,2-c],2′-f][1,3,2]diazaborinin-4-ium-5-uide

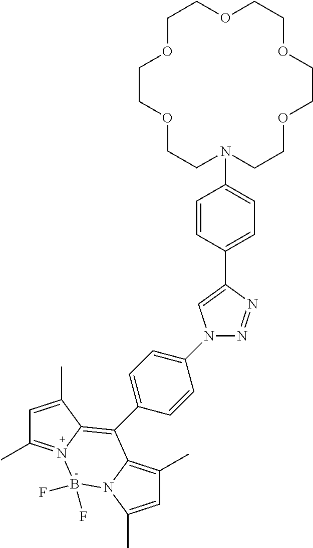

### 10-(4-{1-[4-(1,4,7,10,13-pentaoxa-16-azacyclooctadecan-16-yl)phenyl]-1H-1,2,3-triazol-4-yl}phenyl)-5,5-difluoro-1,3,7,9-tetramethyl-5H-dipyrrolo[1,2-c:1′,2′-f][1,3,2]diazaborinin-4-ium-5-uide

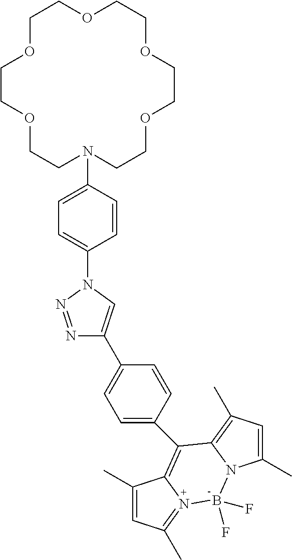

### 5,5-difluoro-10-(4-{4-[3-(2-methoxyethoxy)-4-(1,4,7,10,13-pentaoxa-16-azacyclooctadecan-16-ylphenyl]-1H-1,2,3-triazol-1-yl)phenyl\-1,3,7,9-tetramethyl-5H-dipyrrolo[1,2-c:1′,2′-f][1,3,2]diazaborinin-4-ium-5-uide

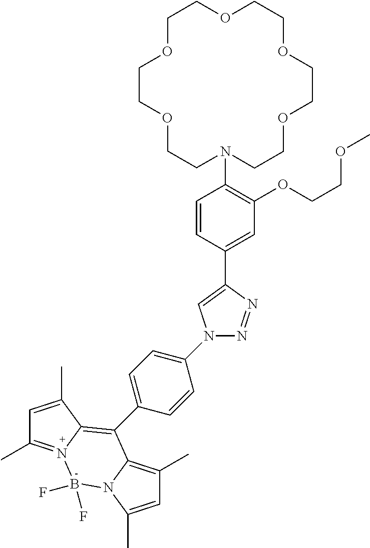

### 5,5-difluoro-10-(4-{1-[3-(2-methoxyethoxy)-4-(1,4,7,10,13-pentaoxa-16-azacyclooctadecan-16-yl)phenyl]-1H-1,2,3-triazol-4-yl}phenyl)-1,3,7,9-tetramethyl-5H-dipyrrolo[1,2-c:1′,2′-f][1,3,2]diazaborinin-4-ium-5-uide

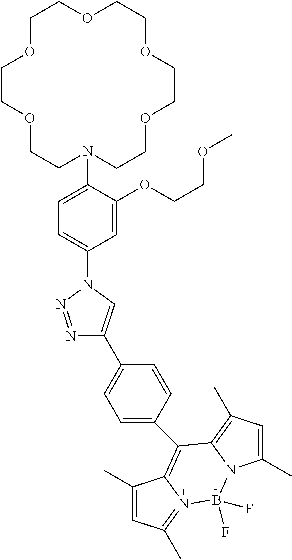

### 5,5-difluoro 10-(4-{4-[3-(2-methoxyethoxy)-4(bis(4-methylbenzo[5,6,17, 181(O,N,N′,O′)-]-1,7,16, -triaza-cryptand-[3,2,2]-phen-4-yl]-1H-1.2.3-triazol 1-yl}1,3,7,9-tetramethyl-5H-dipyrrolo)(1,2-c:1′,2′-f)(1,3,2)diazaborinin-4-ium-5-uide

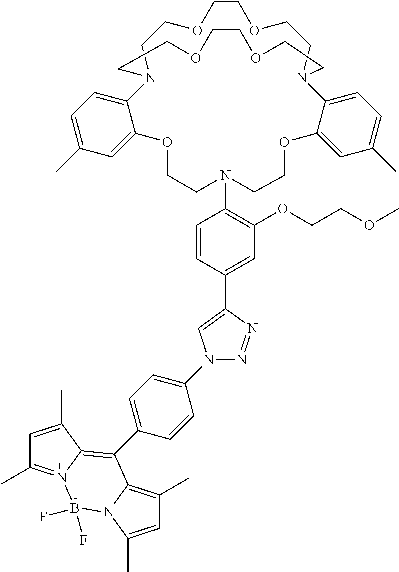

### 5,5-difluoro 10-(4-{1-[3-(2-methoxyethoxy)-4(bis(4-methylbenzo[5,6,17, 18](O,N,N′,O′)-]-1,7,16, -triaza-cryptand-[3,2,2]-phen-4-yl]-1H-1.2.3-triazol 4-yl}1,3,7,9-tetramethyl-5H-dipyrrolo)(1,2-c:1′,2′4)(1,3,2)diazaborinin-4-ium-5-uide

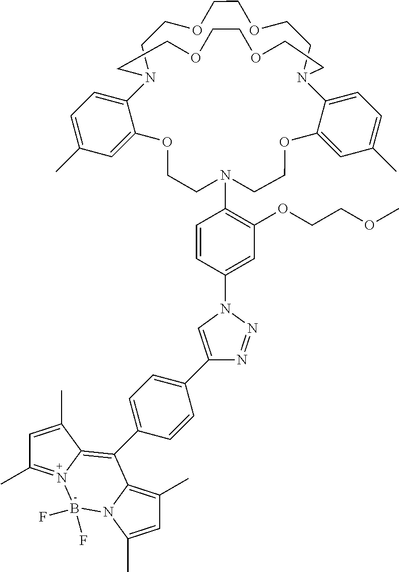

### Sodium Selective

### 10-(4-{4-[4-(1,4,7,10-tetraoxa-13-azacyclopentadecan-13-yl)phenyl]-1H-1,2,3-triazol-1-yl}phenyl)-5,5-difluoro-1,3,7,9-tetramethyl-5H-dipyrrolo[1,2-c:1′,2′-f][1,3,2]diazaborinin-4-ium-5-uide

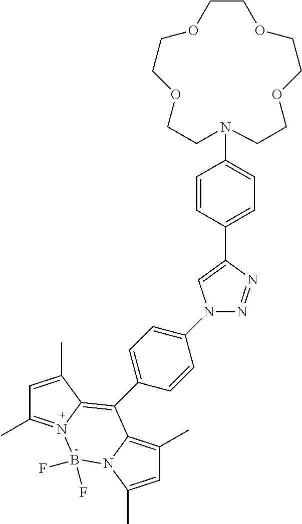

### 10-(4-{1-[4-(1,4,7,10-tetraoxa-13-azacyclopentadecan-13-yl)phenyl]-1H-1,2,3-triazol-4-yl}phenyl)-5,5-difluoro-1,3,7,9-tetramethyl-5H-dipyrrolo[1,2-c],2′-f][1,3,2]diazaborinin-4-ium-5-uide

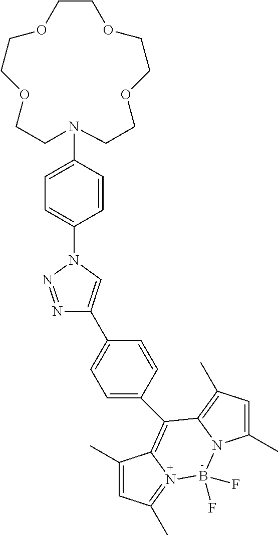

### 5,5-difluoro-10-(4-{4-[3-methoxy-4-(1,4,7,10-tetraoxa-13-azacyclopentadecan-13-yl)phenyl]-1H-1,2,3-triazol-1-yl}phenyl)-1,3,7,9-tetramethyl-5H-dipyrrolo[1,2-c:1′,2′-f][1,3,2]diazaborinin-4-ium-5-uide

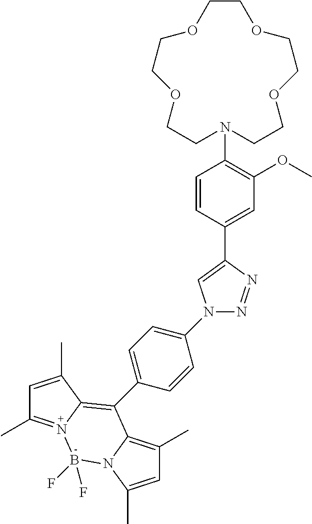

### 5,5-difluoro-10-(4-{1-[3-methoxy-4-(1,4,7,10-tetraoxa-13-azacyclopentadecan-13-yl)phenyl]-1H-1,2,3-triazol-4-yl}phenyl)-1,3,7,9-tetramethyl-5H-dipyrrolo[1,2-c:1′,2′-f][1,3,2]diazaborinin-4-ium-5-uide

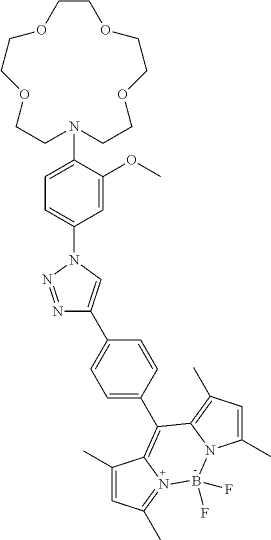

### 5,5-difluoro 10-(4-[3-methoxy-4{bis(4-methylbenzo[5,6,17,18](O,N,N′,O′)-1-1,7,16, -triaza-cryptand-[3,2,1]-phen-4-yl]-1H-1.2.3-triazol-1-yl}-(1,3,7,9-tetramethyl-5H-dipyrrolo)(1,2-c:1′,2′4)(1,3,2)diazaborinin-4-ium-5-uide

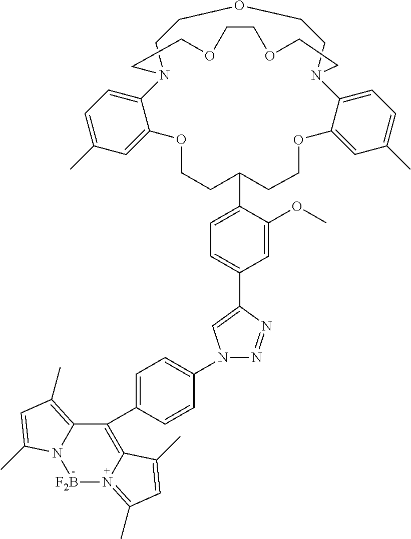

### 5,5-difluoro 10-(1-[3-methoxy-4{bis(4-methylbenzo[5,6,17,18](O,N,N′,O′)-]-1,7,16, -triaza-cryptand-[3,2,1]-phen-1-yl]-1H-1.2.3-triazol-1-yl}-(1,3,7,9-tetramethyl-5H-dipyrrolo)(1,2-c:1′,2′4)(1,3,2)diazaborinin-4-ium-5-uide

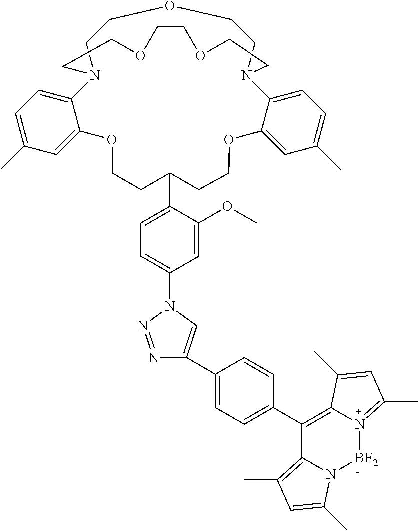

A further object of the present invention is a complex, consisting of a compound according to present invention as given in general Formula (I) and an alkali metal cation. The compounds of the present invention comprise an ionophore that has the capability to bind metal ions. This capability is given by the crown ether or the cryptand moiety of the compounds according to the invention.

According to the invention a complex is preferred, wherein the alkali metal cation is selected from the group consisting of Na⁺ and K⁺.

An object of the present invention is also a method for the determination of metal cations in a sample, comprising the steps of

a) contacting the metal cations with at least one compound according to the present invention,
b) forming a complex according to another object of the invention, whereupon fluorescence and/or luminescence appears or changes,
c) measuring the resulting fluorescence and/or luminescence.

According to the invention a method is preferred, wherein the metal cations are Na⁺ or K⁺, or a combination thereof.

According to the invention a method is especially preferred, wherein the at least one compound according to the invention selectively complexes the metal cations to be determined.

Lastly, another object of the invention is the use of a compound according to the invention in a method according to the invention for the quantitative determination of metal cations in a sample.

### BRIEF DESCRIPTION OF THE FIGURES

The figures show fluorescence spectra of preferred embodiments of the present invention.

FIG. 1 Fluorescence spectra of 1 in MeCN (bottom line) upon addition of 0-2.5 mM NaPF₆ (middle line) and 0-1.16 mM KPF₆ (top line), respectively.

FIG. 2 Fluorescence measurements of 1 (λₑₓ=424 nm) under simulated physiological conditions [180-0 mM choline chloride, 2 mM Ca²⁺, 2 mM Mg²⁺, pH=7.2 (10 mM Tris)]. a) fluorescence spectra in the presence of 0-160 mM Na⁺ (bottom lines) and K⁺(top lines), respectively and b) corresponding FEF with error bars in the presence of 0-160 mM K⁺ or Na⁺ after repeating the experiment with n=4.

FIG. 3 K⁺ Sensitivity of 1 (λₑₓ=424 nm) under simulated physiological conditions, 0-2000 mM KCl, saturation at 1000 mM.

FIG. 4 K⁺ Sensitivity of 5 (λₑₓ=424 nm) under simulated physiological conditions [180-0 mM choline chloride, 2 mM Ca²⁺, 2 mM Mg²⁺, pH=7.2 (10 mM Tris)] in the presence of 0-160 mM K.

FIG. 5 Fluorescence measurements of 5 (λₑₓ=421 nm) under simulated physiological conditions [180-0 mM choline chloride, 2 mM Ca²⁺, 2 mM Mg²⁺, pH=7.2 (10 mM Tris)]. a) fluorescence spectra in the presence of 0-160 mM KCl and 180-0 mM NaCl, respectively and b) corresponding FEF in the presence of 0-160 mM K⁺ or Na⁺.

FIG. 6 K⁺ Sensitivity of 6 (λₑₓ=367 nm) under simulated physiological conditions [180-0 mM choline chloride, 2 mM Ca²⁺, 2 mM Mg²⁺, pH=7.2 (10 mM Tris)] in the presence of 0-160 mM KCl.

FIG. 7 Fluorescence measurements of 6 (λₑₓ=367 nm) under simulated physiological conditions [180-0 mM choline chloride, 2 mM Ca²⁺, 2 mM Mg²⁺, pH=7.2 (10 mM Tris)]. a) fluorescence spectra in the presence of 0-160 mM KCl and 180-0 mM NaCl, respectively and b) corresponding FEF in the presence of 0-160 mM K⁺ or Na⁺.

FIG. 8 K⁺ Sensitivity of 14 (λₑₓ=493 nm) under simulated physiological conditions [180-0 mM choline chloride, 2 mM Ca²⁺, 2 mM Mg²⁺, pH=7.2 (10 mM Tris)] in the presence of 0-180 mM Na⁺.

FIG. 9 Fluorescence measurements of 14 (λₑₓ=430 nm) under simulated physiological conditions [180-0 mM choline chloride, 2 mM Ca²⁺, 2 mM Mg²⁺, pH=7.2 (10 mM Tris)], fluorescence spectra in the presence of 0-180 mM K⁺ plus Na⁺.

FIG. 10 K⁺ Sensitivity of 15 (λₑₓ=493 nm) under simulated physiological conditions [180-0 mM choline chloride, 2 mM Ca²⁺, 2 mM Mg²⁺, pH=7.2 (10 mM Tris)] in the presence of 0-180 mM Na⁺.

FIG. 11 Fluorescence measurements of 15 (λₑₓ=422 nm) under simulated physiological conditions [180-0 mM choline chloride, 2 mM Ca²⁺, 2 mM Mg²⁺, pH=7.2 (10 mM Tris)], fluorescence spectra in the presence of 0-180 mM K⁺ plus Na⁺.

### DETAILED DESCRIPTION OF THE INVENTION

As used herein, the terms have the following meaning:

The term “alkyl” or “lower alkyl” as used herein refers to a straight or branched chain, saturated hydrocarbon having the indicated number of carbon atoms. For example, (C1-C6) alkyl is meant to include, but is not limited to methyl, ethyl, propyl, isopropyl, butyl, sec-butyl, tert-butyl, pentyl, isopentyl, neopentyl, hexyl, isohexyl, and neohexyl. An alkyl or lower alkyl group can be unsubstituted or optionally substituted with one or more substituents.

The term “alkoxy” or “lower alkoxy” as used herein refers to an —O-alkyl group having the indicated number of carbon atoms. For example, a (C1-C6) alkoxy group includes —O-methyl, —O-ethyl, —O-propyl, —O-iospropyl, —O-butyl, —O-sec-butyl, —O-tert-butyl, —O-pentyl, —O-isopentyl, —O-neopentyl, —O-hexyl, —O-isohexyl, and —O-neohexyl.

The term “aromatic” as used herein refers to an aromatic or heteroaromatic moiety. An “aromatic” moiety refers to a 6- to 14-membered monocyclic, bicyclic or tricyclic aromatic hydrocarbon ring system. Examples of an aromatic group include phenyl and naphthyl. An aromatic group can be unsubstituted or optionally substituted with one or more substituents. The term “heteroaromatic” as used herein refers to an aromatic heterocyclic ring of 5 to 14 members and having at least one heteroatom selected from nitrogen, oxygen and sulfur, and containing at least 1 carbon atom, including monocyclic, bicyclic, and tricyclic ring systems. Representative heteroaromatics are triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, pyrimidyl, azepinyl, oxepinyl, naphthothiazolyl, quinoxalinyl. A heteroaromatic group can be unsubstituted or optionally substituted with one or more substituents.

The term “halogen” as used herein refers to —F, —Cl, —Br or —I.

As used herein, the term “heteroatom” is meant to include oxygen (O), nitrogen (N), and sulfur (S).

As used herein, the term “BODIPY” is an acronym for the compound class of boron-d ipyrromethanes.

The new π-conjugated 1,2,3-triazol-1,4-diylfluoroionophores according to the present invention generated via Cu(I) catalyzed [3+2] cycloaddition show high fluorescence enhancement factors in the presence of Na⁺ and K⁺ in MeCN and high selectivity towards Na⁺ and K⁺ under simulated physiological conditions.

Preferred embodiments of the present invention are compounds 1 and 2 as given in Scheme 1. The inventors found that in the simple CuAAC-generated fluoroionophore 1, the electronic conjugation of the N-phenylaza-18-crown-6 ether and the 7-diethylaminocoumarin fluorophore through a 1,2,3-triazol-1,4-diyl π-linker results in a perfect signal transduction chain for the sensing of Na⁺ and K⁺. In MeCN high cation-induced FEFs were obtained for 1 (FEF_{Na+}: 58; FEF_{K+}: 27). The signal transduction in 1 also works nicely under simulated physiological conditions. In the presence of 160 mM K⁺ a FEF of 2.5 was observed, whereas the same concentration range of Na⁺ resulted in an almost negligible fluorescence enhancement. The inventors assume, that compound 1 is a PET-fluoroionophore with a virtual spacer between the anilinotriazole electron donor unit and the coumarin electron acceptor moiety.

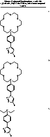

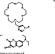

The CuAAC of the ethinyl-functionalized N-phenylaza-18-crown-6 ether⁷ with 3-azido-7-diethylaminocoumarin⁸ afforded 1,2,3-triazole-fluoroionophore 1. The triazole-isomer 2 was obtained by the reaction of the azido-functionalized N-phenylaza-18-crown-6 ether⁹ with 3-ethinyl-7-diethylamino-coumarin¹⁰. Dye 3, in which the crown has been replaced by a diethylamino group was synthesised in order to represent a reference compound for 1. A further reference compound for 1 is compound 4, in which the p-phenylen linker between aza-18-crown-6 and triazole ring is replaced by a deconjugated methylene group. Compound 4 was obtained by the reaction of N-propargylaza-18-crown-6 ether¹¹ with 3-azido-7-diethyl-aminocoumarin. The new 1,4-disubstituted 1,2,3-triazoles 1-4 are stable in solutions of MeCN and DMSO at room temperature for many weeks. UV-irradiation over a period of several hours did not show any decomposition of the fluoroionophores 1 and 2.

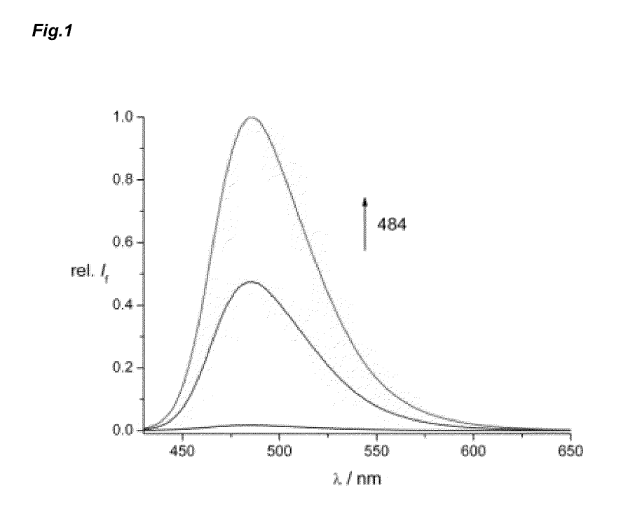

The fluorescence quantum yield of 1 in MeCN is extremely low (Φ_{f}(1)=0.008, Table 1). The fluorescence spectra of 1 in the presence of Na⁺ and K⁺, respectively (FIG. 1) show the impressive fluorescence enhancement upon coordination of the alkali metal ions. High FEFs could be determined for Na⁺ (FEF=58) and for K⁺ (FEF=27).¹²

TABLE 1
Photophysical properties of 1-4 in MeCN.

| ligand | 1 | 2 | 3 | 4 |
|---|---|---|---|---|
| λₑₘ/nm^{a} | 484 | 470 | 493 | 480 |
| λₑₓ/nm^{b} | 410 | 412 | 410 | 408 |
| Φ_{f}(ligand)^{c} | 0.008 | 0.03 | 0.017 | 0.56 |
| Φ_{f}(ligand + Na⁺)^{d} complex)^{d} | 0.62 | 0.79 | 0.018 | 0.55 |
| Φ_{f}(ligand + K⁺)^{d} | 0.23 | 0.40 | 0.016 | 0.58 |

^{a}Emission maxima,
^{b}excitation maxima,
^{c}fluorescence quantum yields (Φ_{f}),
^{d}Φ_{f} were determined in the presence of 40 mM of NaPF₆ or 40 mM KPF₆, respectively.

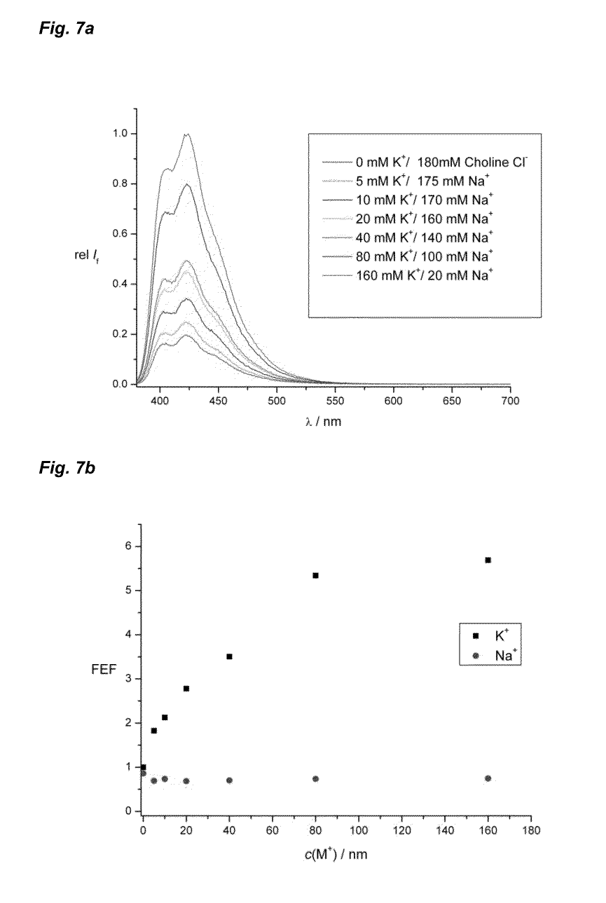

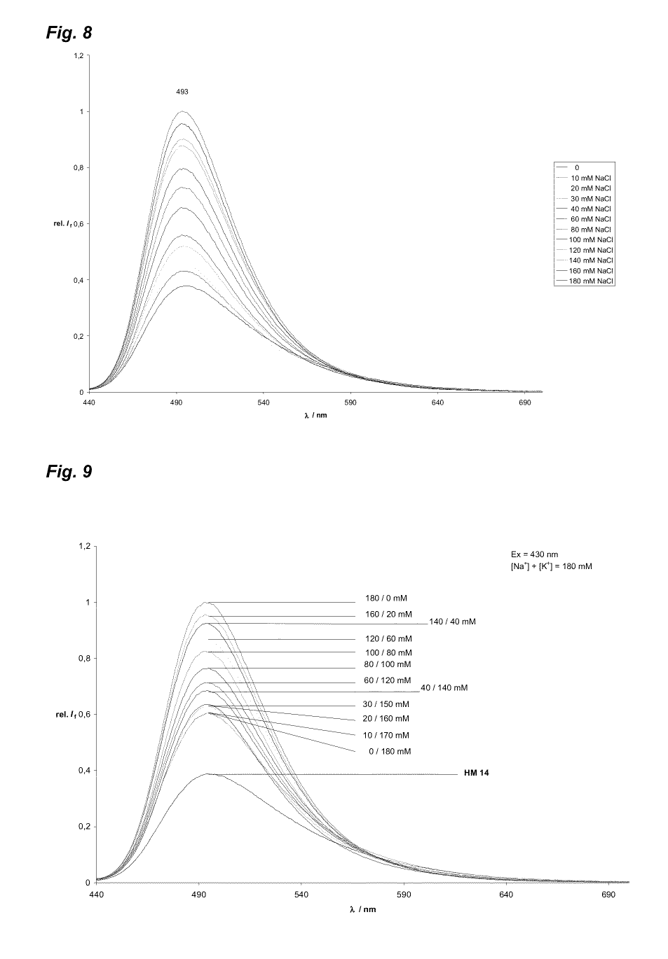

The fluorescence quantum yield of the constitutional isomer 2 in MeCN is higher than that of 1 (Φ_{f}(2)=0.03, Table 1). The fluorescence of 2 is also enhanced in the presence of increasing concentrations of Na⁺ and K⁺ (FIGS. 7 and 8, respectively). However the observed FEFs for Na⁺ (FEF=17) and K⁺ (FEF=13) are significantly lower than for the isomer 1.

The reference compound 4 consists of a poorer PET donor due to the aliphatic amine which is electronically separated from the triazole. Hence 4 has a high quantum yield (Φ_{f}(4)=0.56, Table 1) and is therefore less affected by the presence of Na⁺ and K⁺.

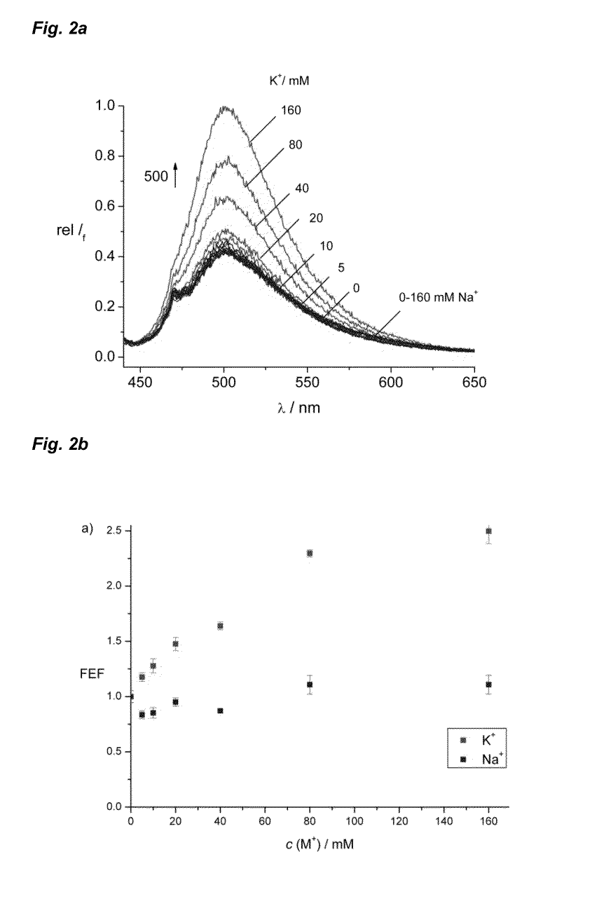

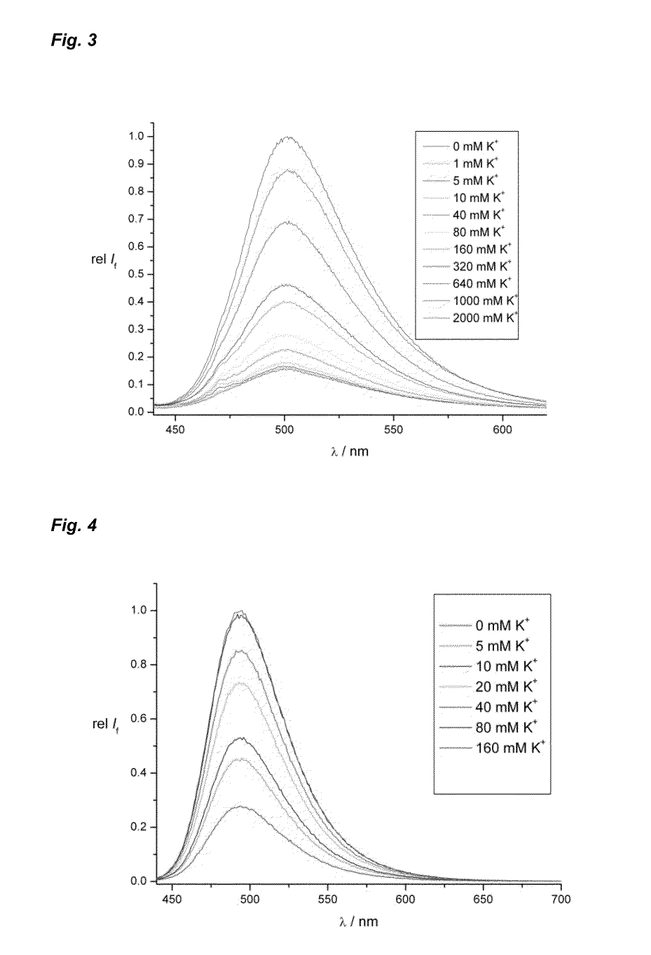

The inventors further investigated the influence of Na⁺ and K⁺ on the fluorescence of 1 under simulated physiological conditions. The ligand was exposed to aqueous solutions in the physiological interesting concentration range of 0-160 mM Na⁺ or K⁺, respectively. Additionally, the solutions contained 2 mM Ca²⁺ and 2 mM Mg²⁺. The pH was adjusted to 7.2 with 10 mM Tris and a constant ionic strength of 180 mM was maintained with choline chloride. Under these conditions, the fluorescence of 1 (λex=424 nm, λem=500 nm, Φ_{f}=0.07) is hardly increased in the presence of Na⁺ whereas increasing concentrations of K⁺ resulted in a modest fluorescence enhancement (FIG. 2a). In the presence of 160 mM K⁺ a FEF of 2.5 is observed. The fluorescence measurements were repeated four times showing only very little variations in the FEF values (FIG. 2b). It is noteworthy, that the presence of physiological important extracellular cations, such as Mg²⁺ and Ca²⁺ does not affect the signal response.

In comparison, the FEF of 1 for K⁺ in MeCN is clearly smaller than the FEF in water under simulated physiological conditions. This can be explained by the significantly smaller stability constants of the K⁺ complex with the N-phenylaza-18-crown-6 in water [IgK (H₂O)>0.5; IgK (MeCN)=3.95±0.08]. The fact that the fluorescence of 1 in water is exclusively enhanced in the presence of K⁺ and not by Na⁺ can be rationalized by the stronger hydration enthalpy of Na⁺. However, the fluorescence enhancement of 1 in the presence of K⁺ under simulated physiological conditions shows that the signaling transduction chain in this fluoroionophore works well in water.

The dissociation constant (Kd) of 1+K⁺ amounts to ˜260 mM in solutions which approximates physiological ionic strength. To measure intracellular or extracellular concentrations of K⁺ (Kd around 140 or 4 mM) tuning of probe 1 towards a higher complex stability while maintaining the selectivity will be necessary.

To investigate the pH-sensitivity of 1, the fluorescence intensity was measured in water at different pH values. The resulting pKa of 1 is near 4.5 meaning that the triazole-substituted N-phenylaza-18-crown-6 is less pH-sensitive than the N(o-methoxyphenyl)aza-15-crown-5-naphthalimide Nat fluoroionophore (pKa ˜5.5) of the authors He et al.¹³

In summary, the inventors have shown for the first time that an electronically conjugated 1,2,3-triazole-fluoroionophore consisting of the signaling transduction chain: anilino-ionophore-1,2,3-triazol-1,4-diyl-fluorophore, works as an effective sensor for Na⁺ and K⁺ in acetonitrile with high cation-induced FEFs. Under simulated physiological conditions 1 selectively detects K⁺with the modest FEF being limited by the rather simple receptor unit. The inventors found that the substitution of ionophore and fluorophore on the 1,2,3-triazole ring has a basic influence on the quality of the fluoroionophore.

Recently He et al.^{13a} and the Verkman group^{13b} designed fluorescence switch-on PET sensors with high K⁺/Na⁺ selectivity and sensitivity for physiological K⁺ in the concentration range of 0-40 mM. In these K⁺fluoroionophores a [3.2.2]-cryptand represents the ionophore. A drawback of the [3.2.2]-cryptand fluoroionophores though, is the very expensive synthetic procedure.^{13c}

The inventors developed simple lariat aza-18-crown-6 ionophores, which show a high K⁺/Na⁺ selectivity and sensitivity under simulated physiological conditions. These novel lariat aza-18-crown-6 ionophores are also an object of the present invention.

The further preferred embodiments of the compounds of the present invention containing a 3(2-methoxyethoxy)phenyl-aza-18-crown-6 as K⁺/Na⁺ selective and sensitive ionophore are given in Scheme 3.

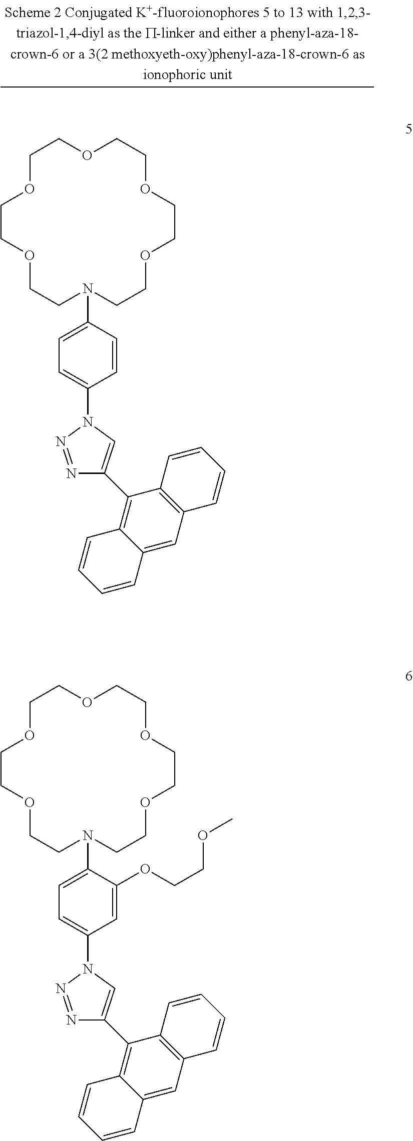 [SMILES: c1ccc2c(-c3cn(-c4ccc(N5CCOCCOCCOCCOCCOCC5)cc4)nn3)c3ccccc3cc2c1 ; COCCOc1cc(-n2cc(-c3c4ccccc4cc4ccccc34)nn2)ccc1N1CCOCCOCCOCCOCCOCC1]

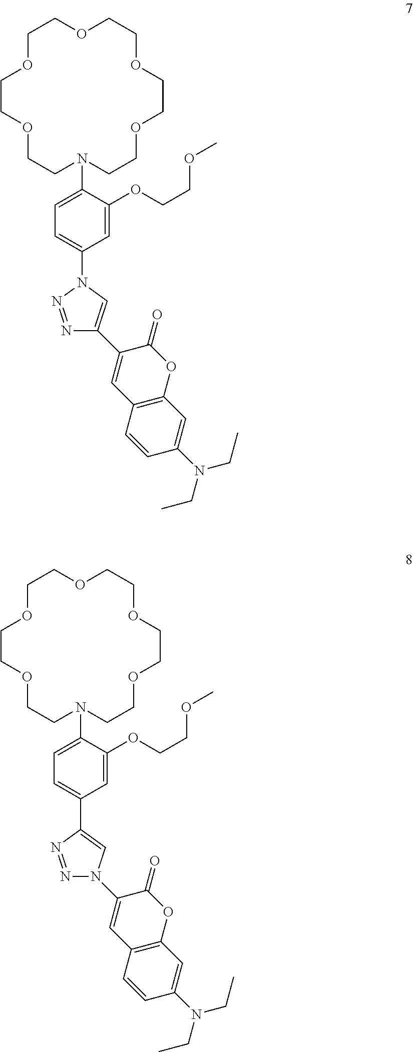 [SMILES: CCN(CC)c1ccc2cc(-c3cn(-c4ccc(N5CCOCCOCCOCCOCCOCC5)c(OCCOC)c4)nn3)c(=O)oc2c1 ; CCN(CC)c1ccc2cc(-n3cc(-c4ccc(N5CCOCCOCCOCCOCCOCC5)c(OCCOC)c4)nn3)c(=O)oc2c1]

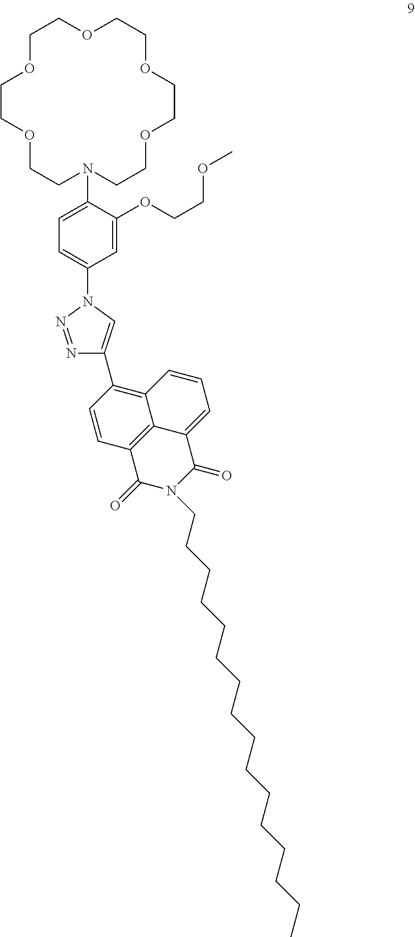 [SMILES: CCCCCCCCCCCCCCCCN1C(=O)c2cccc3c(-c4cn(-c5ccc(N6CCOCCOCCOCCOCCOCC6)c(OCCOC)c5)nn4)ccc(c23)C1=O]

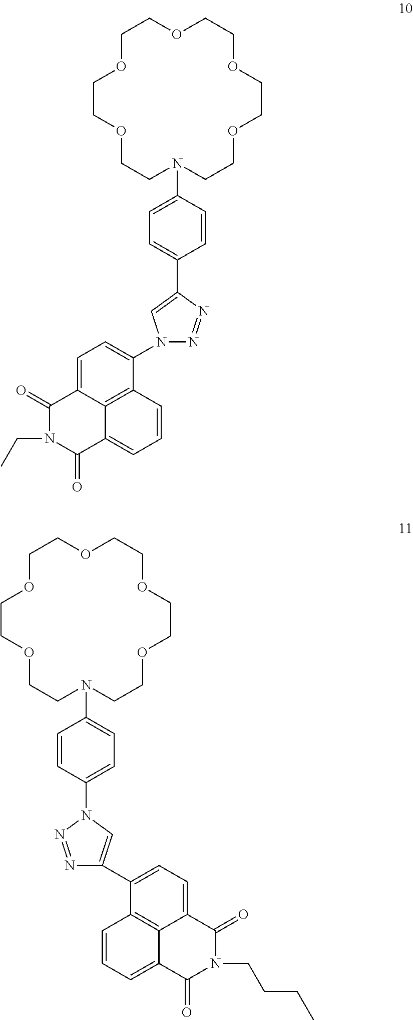 [SMILES: CCN1C(=O)c2cccc3c(-n4cc(-c5ccc(N6CCOCCOCCOCCOCCOCC6)cc5)nn4)ccc(c23)C1=O ; CCCCN1C(=O)c2cccc3c(-c4cn(-c5ccc(N6CCOCCOCCOCCOCCOCC6)cc5)nn4)ccc(c23)C1=O]

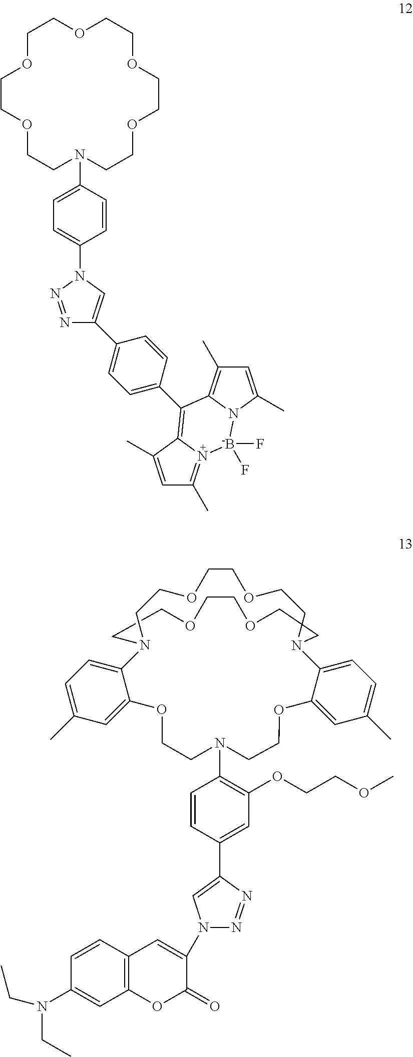

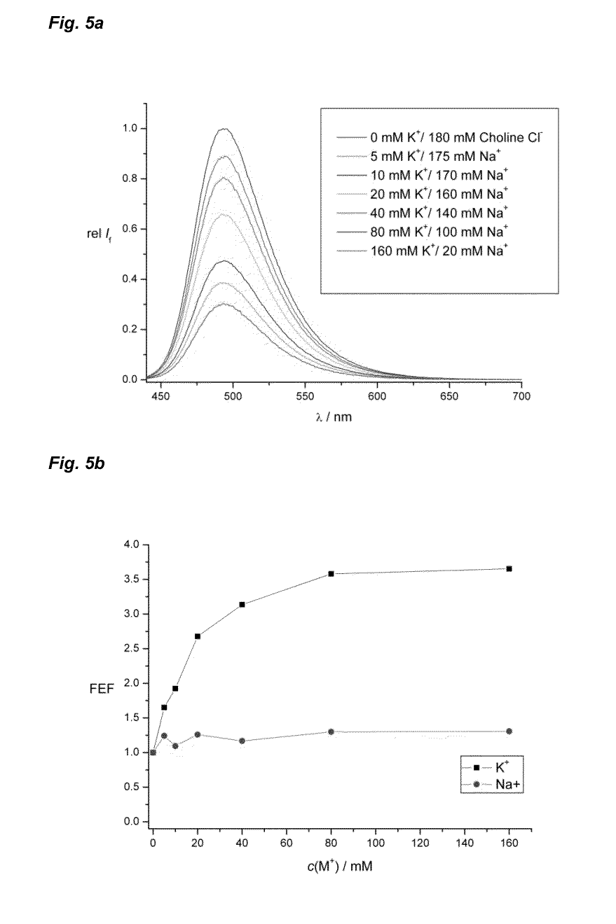

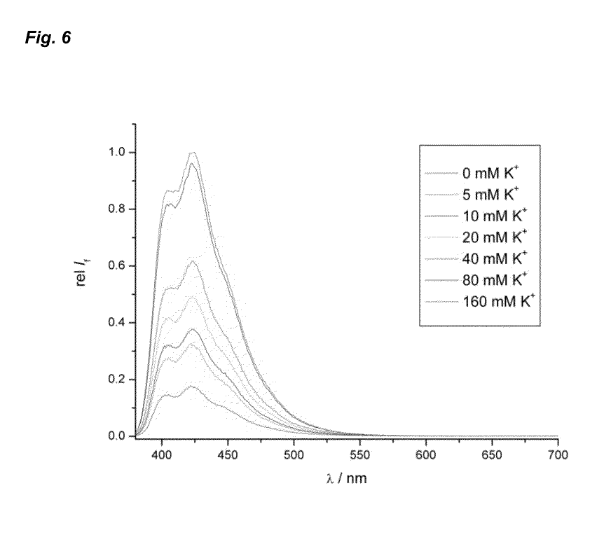

Compounds 6 to 9 have a 2-methoxyethoxy-lariate chain in close proximity to the aza-18-crown-6. This lariate group provides a higher binding selectivity for K. The fluorescence spectra are shown in FIGS. 4 to 7.

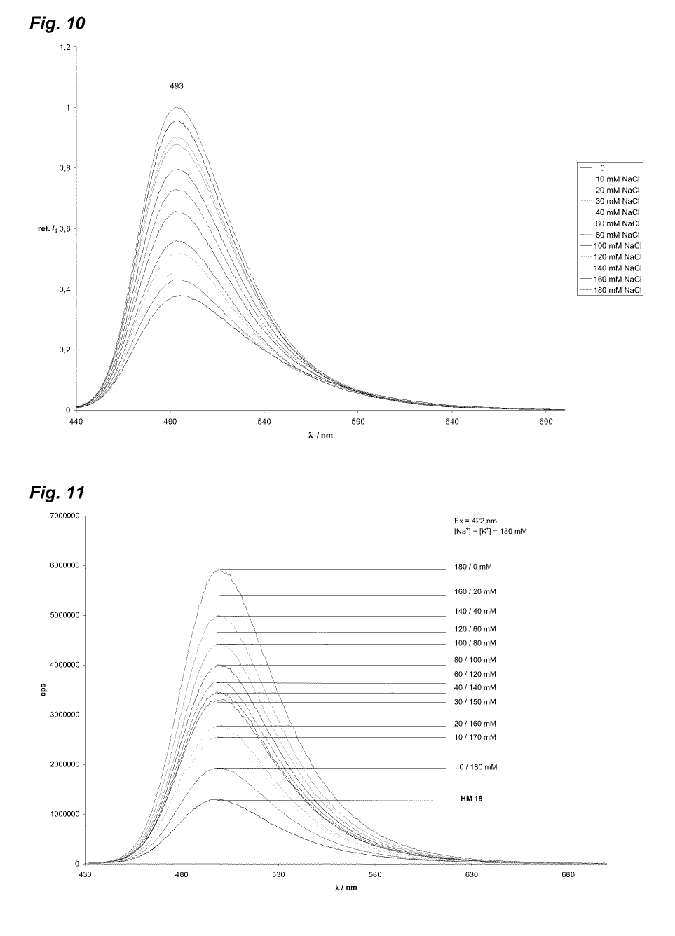

According to the invention Na⁺-fluoroionophores 14 and 15 with a 1,2,3-triazol-1,4-diyl as the π-linker (Scheme 4) are provided. These fluoroionophores show high Na⁺/K⁺-selectivity under simuated physiological conditions. The fluorescence spectra are shown in FIGS. 8 to 11.

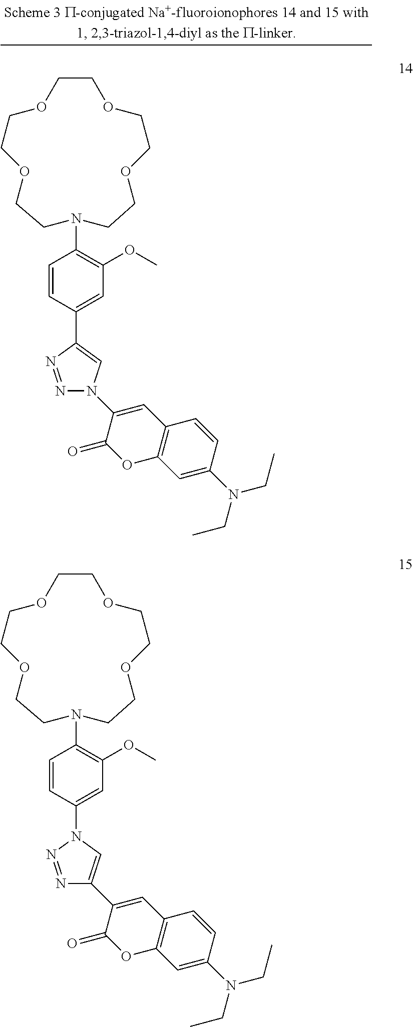 [SMILES: CCN(CC)c1ccc2cc(-n3cc(-c4ccc(N5CCOCCOCCOCCOCC5)c(OC)c4)nn3)c(=O)oc2c1 ; CCN(CC)c1ccc2cc(-c3cn(-c4ccc(N5CCOCCOCCOCCOCC5)c(OC)c4)nn3)c(=O)oc2c1]

The following examples explain the present invention in more detail. The embodiments described in the examples are not limiting the scope of the invention. The examples should only serve as preferred embodiments and a skilled artesian will derive other embodiments from those examples without any undue burden.

### EXAMPLES

### Fluorescence Measurements

Fluorescence titration spectra of the compounds (c=5·10⁻⁶ mol·l⁻¹) in acetonitrile were recorded 5 min after the addition of 0.02 ml of volumetric standard solution of NaPF₆ or KPF₆ (c=5′10⁻⁵-5′10⁻² mol·l⁻¹), respectively. Titration was continued until no change in fluorescence enhancement was observed. Fluorescence Quantum yields were determined using a PL Quantum Yield measurement System C9920-2 of Hamamatsu, Japan.

Fluorescence spectra in aqueous solutions were carried out in buffered saline solutions (10 mm Tris-buffer, pH=7.2) using a 1 mM ligand solution in DMSO. The water used was purified by a Milli-Q-Deioniser from Millipore®. To obtain physiological conditions, the salt solutions contained constant concentrations of CaCl₂ (2 mM) and MgCl₂ (2 mM). The physiological solutions were varied with regard to the concentrations of respectively KCl or NaCl and constant ionic strength was adjusted to a typical ionic strength in physiological systems (180 mM) by choline chloride.

The dye was exposed to different saline solutions containing 0, 5, 10, 20, 40, 80, 160 mM KCl or NaCl, respectively (Table 2 and 3). Each salt solution was mixed with 1 mm DMSO solution of the compound (990/10 v/v) to give a final ligand concentration of 10 μm. For each measurement, a freshly prepared dye mixture was used and every experiment was repeated with n=4. Fluorescence response of the compounds was recorded exciting at the given wavelengths.

TABLE 2
Composition of the different aqueous solutions under simulated
physiological conditions with regard to K⁺- selectivity.

| KCl/mM | Choline Cl/mM | MgCl₂/mM | CaCl₂/mM |
|---|---|---|---|
| 0 | 180 | 2 | 2 |
| 5 | 175 | 2 | 2 |
| 10 | 170 | 2 | 2 |
| 20 | 160 | 2 | 2 |
| 40 | 140 | 2 | 2 |
| 80 | 100 | 2 | 2 |
| 160 | 20 | 2 | 2 |

TABLE 3
Composition of the different aqueous solutions under simulated
physiological conditions with regard to Na⁺- selectivity.

| NaCl/mM | Choline Cl/mM | MgCl₂/mM | CaCl₂/mM |
|---|---|---|---|
| 0 | 180 | 2 | 2 |
| 5 | 175 | 2 | 2 |
| 10 | 170 | 2 | 2 |
| 20 | 160 | 2 | 2 |
| 40 | 140 | 2 | 2 |
| 80 | 100 | 2 | 2 |
| 160 | 20 | 2 | 2 |

### General Methods and Procedures of Preparation of Compounds

All commercially available chemicals were used without further purification. Solvents were distilled prior use. ¹H and ¹³C NMR spectra were recorded on 300 MHz, 500 MHz or 600 MHz instruments. Data are reported as follows: chemical shifts in ppm (δ), multiplicity (s=singlet, d=doublet, t=triplet, m=multiplet, dd=doublet of doublets), integration, coupling constant (Hz). ESI spectra were recorded using a Micromass Q-TOF micro mass spectrometer in a positive electrospray mode. IR spectra were recorded using a Thermo Nicolet NEXUS FTIR instrument.

Air/water-sensitive reactions were performed in oven-dried glassware under an argon atmosphere. Column chromatography was performed with SiO₂ (Merck Silica Gel 60 (0.04-0.063 mesh)).

### Preparation of Precursors

The preparation of 3-azido-7-diethylaminocoumarine (60),¹⁴ 3-ethinyl-7-diethylaminocoumarine (61),¹⁵ N-phenylaza-18-crown-6 ether¹⁶ and N-(4-formyl)phenylaza-18-crown-6 ether¹⁷ has been described previously.

### 16-(4-ethynylphenyl)-1,4,7,10,13-pentaoxa-16-azacyclooctadecane (27)

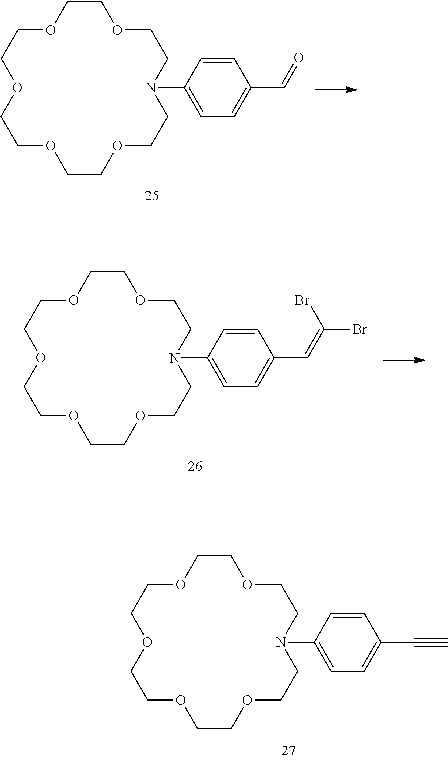 [SMILES: O=Cc1ccc(N2CCOCCOCCOCCOCCOCC2)cc1 ; BrC(Br)=Cc1ccc(N2CCOCCOCCOCCOCCOCC2)cc1 ; C#Cc1ccc(N2CCOCCOCCOCCOCCOCC2)cc1]

### N-4-(2,2-Dibromovinyl)phenylaza-18-crown-6 ether (26)

The preparation of 27 followed the literature and references therein according to a modified procedure.¹⁸

A suspension of zinc (1.69 g, 25.9 mmol) and carbon tetrabromide (8.59 g, 25.9 mmol) in dry CH₂Cl₂ (40 ml) was cooled to −15° C. with an ice-salt bath before a solution of triphenylphosphine (6.79 g, 25.9 mmol) in dry CH₂Cl₂ was added dropwise. The resulting yellow green mixture was kept at −15° C. for 30 min and was then stirred at RT for 3 h whereupon a solution of 25 (4.31 g, 11.7 mmol) in dry CH₂Cl₂ was added dropwise. The red brown mixture was stirred at RT for 2 h and then water (100 ml) was added. The organic phase was separated and washed with water (3×75 ml).The combined organic layer were dried with MgSO₄ and concentrated to give 26 as a light brown oil that was directly used for the next step.

Note that the product decomposes when concentrated to dryness resulting in a green solid. Storage at −20° C. is recommended.

HRMS (⁺ESI): m/z calcd for (M+H)⁺, 522.04, 524.05, 526.04. found, 522.11, 524.11, 526.11.

26 (2.91 g, 5.56 mmol) was dissolved in 25 ml dry THF and set under argon atmosphere immediately. It was cooled to −78° C. before n-butyllithium (7.5 ml, 1.6 M, solution in hexane) was added via a syringe. After stirring for 1 h at −78° C. the solution was allowed to warm up to room temperature and stirring was continued at this temperature for 1 h before H₂O (15 ml) was added cautiously. The reaction mixture was extracted with 3×CH₂Cl₂ and the combined organic layers were dried with MgSO₄ and concentrated to give 27 as a red brown oil, which was purified via chromatography (silica gel, CHCl₃/MeOH, 95/5 v/v). Yield: 20%, overall.

¹H NMR (CDCl₃, 300 MHz): δ=2.93 (s, 1H), 3.6 (m, 24H), 6.56 (d, 2H, J=9.23 Hz), 7.28 (d, 2H, J=8.85 Hz); ¹³C NMR (CDCl₃, 75 MHz): δ=51.66, 68.94, 71.15, 75.12, 85.17, 108.82, 111.62, 133.73, 148.45; HRMS (⁺ESI): m/z calcd for (M+H)⁺, 364.21. found, 364.26; IR(ATR, cm⁻¹): 718, 692, 1115, 2097, 2868, 3054.

### 16-(4-azidophenyl)-1,4,7,10,13-pentaoxa-16-azacyclooctadecane (28)

The preparation of the N-(4-nitro)phenylaza-18-crown ether and subsequent hydrogenation to the corresponding amine followed the literature.¹⁹

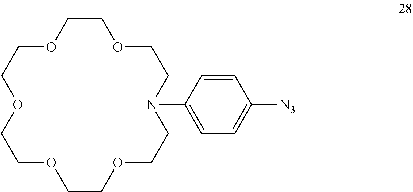 [SMILES: [N-]=[N+]=Nc1ccc(N2CCOCCOCCOCCOCCOCC2)cc1]

The synthesis of 28 followed the literature according to a modified procedure.²⁰ N-Anilino-4-aza-18-crown-6 ether (4.03 g, 11.4 mmol) was dissolved in 90 ml HCl (4M) and cooled to 0° C. A solution of NaNO₂ (0.784 g, 11.4 mmol) in 45 ml H₂0 was added dropwise. The mixture was stirred for 10 min before a solution of NaN₃ (1.1 g, 17 mmol) in 45 ml H₂O was added drop wise. Stirring was continued for another 10 min at 0° C. before the orange solution was allowed to warm up to room temperature and it was stirred for 14 h at ambient temperature. The reaction mixture was brought to pH=7 with Na₂CO₃, was extracted with 3×180 ml CHCl₃ and the combined organic layers were dried with MgSO₄. The solvent was removed under reduced pressure to yield 28 as brown oil (1.31 g, 30.3%). The product was used for the next reaction step without further purification.

¹H NMR (CDCl₃, 300 MHz): δ=3.57-3.69 (m, 24H), 6.68 (d, 2H, J=8.854 Hz), 6.88 (d, 2H, J=9.042 Hz); ¹³C NMR (CDCl₃, 75 MHz): δ=51.48, 68.74, 70.70, 112.97, 119.94, 127.55, 145.98;

HRMS (⁺ESI): m/z calcd for (M+H)⁺, 381. 21. found, 381.34, calcd for (M-N₂)⁺353.21. found, 353.30; IR (ATR, cm⁻¹): 1100, 1508, 2120, 2097, 2867.

### 16-(prop-2-ynyl)-1,4,7,10,13-pentaoxa-16-azacyclooctadecane (29)

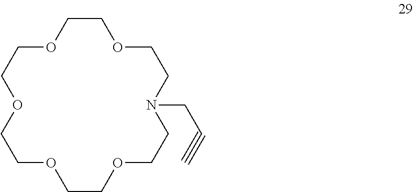 [SMILES: C#CCN1CCOCCOCCOCCOCCOCC1]

The synthesis followed the literature according to a modified procedure.²¹ Monoaza-18-crown-6 ether²² (0.6 g, 2.28 mmol) and propargylbromide (0.206 ml, 2.74 mmol) were dissolved in dry acetonitrile (70 ml) before Cs₂CO₃ (1.48 g, 4.56 mmol) was added. The suspension was stirred overnight at 85° C. After cooling to room temperature, the suspension was filtered and the filtrate was concentrated. The residue was purified by column chromatography on silica gel eluting with CHCl₃/MeOH (9/1 v/v) yielding 29 as a red oil (64%)

¹H NMR (CDCl₃, 300 MHz): δ=2.18 (t, 1H, J=2.45 Hz), 2.82 (t, 4H, J=5.28 Hz), 3.61-3.74 (m, 22H); ¹³C (CDCl₃, 75 MHz): δ=43.42, 53.29, 68.94, 70.52, 73.61, 78.85; IR (ATR, cm⁻¹): 1115, 2867, 3189; HRMS (⁺ESI): m/z calcd. for (M+H)⁺,302.20. found, 302.21.

### N,N-diethyl-4-ethynylaniline (32)

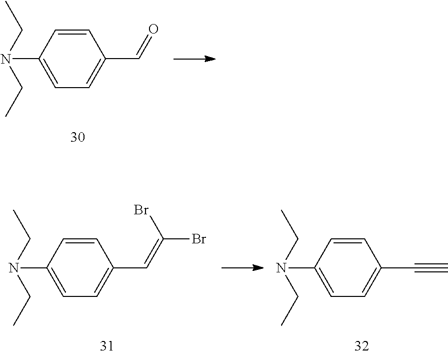

The synthesis followed the literature according to a modified procedure.²³

### 1,1-Dibromo-2-(4-N,N-diethylaminophenyl)ethane (31)

A mixture of zinc (4.9 g, 75 mmol), triphenylphosphine (19.67 g, 75 mmol), and carbon tetrabromide (24.87 g, 75 mmol) in dry dichloromethane (250 ml) was sonicated for 2 h while cooling with an ice bath. N,N-diethylaminobenzaldehyde (30) (5.30 g, 29.9 mmol) was added to the grayish suspension and it was stirred overnight resulting in a brownish suspension. The mixture was concentrated and petroleum ether (500 ml) was added, whereupon a tarry precipitate formed. It was washed with 1/1 CH₂Cl₂/light petroleum ether (2×100 ml). The combined organic phases were concentrated, and the residue was chromatographed on silica gel (150 g), eluting with 1/1 CH₂Cl₂/hexane to give 31 as a yellow oil (7.3 g, 21.0 mmol, 70%). The intermediate product was kept at −20° C. until the next step.

¹H NMR (CDCl₃, 300 MHz): δ=1.17 (t, 6H, J=6.94 Hz, CH₃), 3.37 (q, 4H, J=6.94 Hz, CH₂), 6.62 (d, 2H, J=7.88 Hz, Ar—H), 7.32 (s, 1H, CH), 7.49 (d, 2H, J=8.21 Hz); HRMS (⁺ESI): m/z calcd. for (M+H)⁺, 331.96, 333.96, 335.96. found, 331.93, 333.93, 335.94.

### N,N-diethyl-4-ethynylaniline (32)

A solution of 31 (4.3 g, 12.4 mmol) in dry THF (93 ml) was cooled to −78° C. before n-butyllithium (19 ml, 15% in hexane) was added dropwise. It was stirred for 45 min at this temperature before the reaction mixture was allowed to warm up to RT. After stirring for 1 h at RT, water (7 ml) was added slowly. The solvent was removed and the residue was taken up in diethyl ether. The organic layer was washed with water (30 ml) and brine (30 ml) and dried over MgSO₄. Concentration of the ether phase gave 32 as orange oil that was used in the next step without further purification (1.29 g, 6.96 mmol, 56.2%).

¹H NMR (CDCl₃, 300 MHz): δ=1.16 (t, 6H, J=7.25 Hz), 2.97 (s, 1H), 3.37 (q, 4H, J=7.25 Hz,), 6.57 (d, 2H, J=9.14 Hz), 7.34 (d, 2H, J=9.14 Hz), ¹³C (CDCl₃, 75 MHz): δ=12.46, 44.26, 74.41, 85.01, 107.47, 110.94, 133.39, 147.73, HRMS (⁺ESI): m/z calcd. for (M+H)⁺, 174.13. found, 174.16.

### 13-(4-Ethynyl-2-methoxyphenyl)-1,4,7,10-tetraoxa-13-azacyclopentadecane (40)

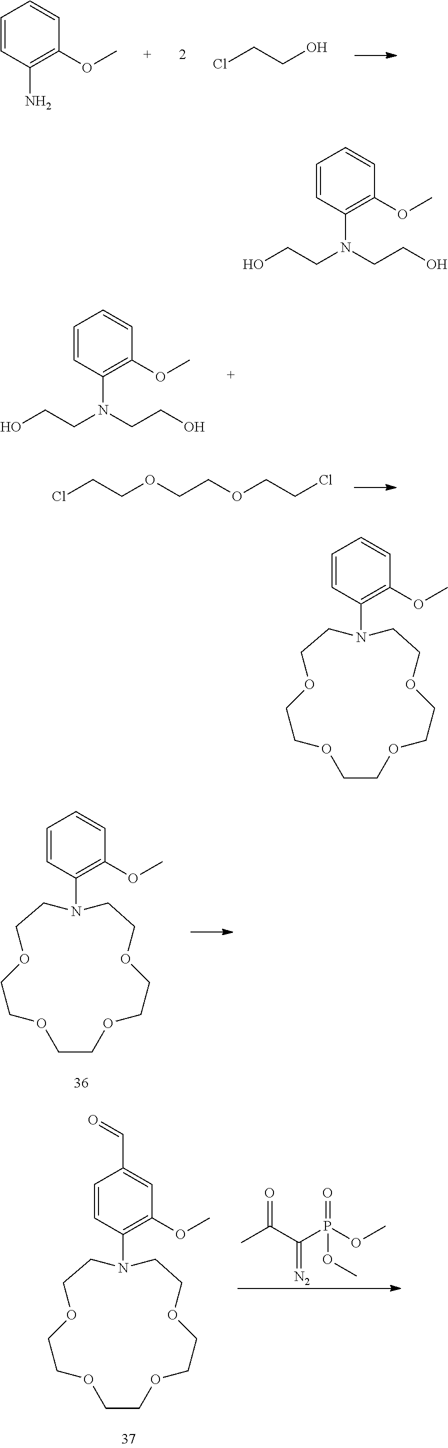

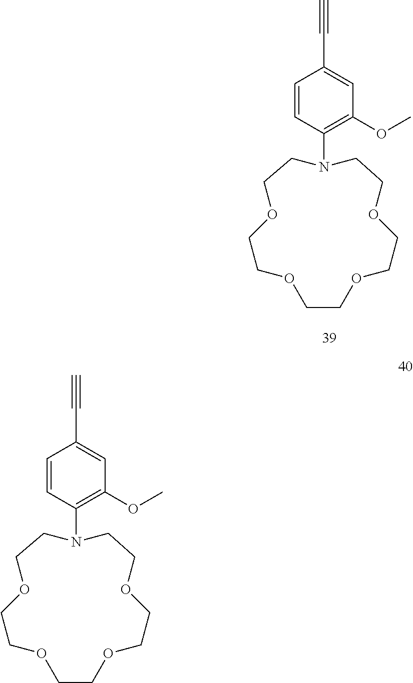 [SMILES: C#Cc1ccc(N2CCOCCOCCOCCOCC2)c(OC)c1 ; C#Cc1ccc(N2CCOCCOCCOCCOCC2)c(OC)c1]

The synthesis followed the literature according to a modified procedure.24

Into a stirred mixture of 860 mg (2.43 mmol) Aldehyd 37 and 673 mg (4.86 mmol) K₂CO₃ in 35 ml dry Methanol was added 564 mg (2.93 mmol) dimethyl-1-diazo-2-oxopropylphosphonate (Bestmann-Ohira Reagent). This mixture was stirred for 20 hours at ambient temperature. After addition of 60 ml CHCl₃ and extraction 3 times with water, the organic layer was separated, dried with MgSO₄ and concentrated in vacuum. The residue was purified by column chromatography on silica with CHCl₃/CH₃OH (95/5) as eluent. The product was obtained as a yellow oil (435 mg, 51%).

¹H NMR (300 MHz, CDCl₃): δ=2.99 (s, 1H), 3.44-3.48 (m, 4H), 3.58-3.66 (m, 16H), 3.77 (s, 3H),

¹³C NMR (75 MHz, CDCl₃): δ=52.85, 55.37, 69.94, 70.25, 70.34, 70.85, 75.57, 84.15, 113.94, 115.13, 119.06, 125.21, 140.93, 151.32

MS (EI) m/z (%): 349(100) [M]⁺.

IR (ATR, cm⁻¹): 2923 (s), 2855 (s), 2102 (m), 1507 (s), 1253 (s), 1110 (s).

Elemental analysis (%) calcd. for C₁₉H₂₇NO₅ (349.42): C, 65.31, H, 7.79, N, 4.01. found C, 65.12; H, 7.38; N, 3.98

### 13-(4-Azido-2-methoxyphenyl)-1,4,7,10-tetraoxa-13-azacyclopentadecane (43)

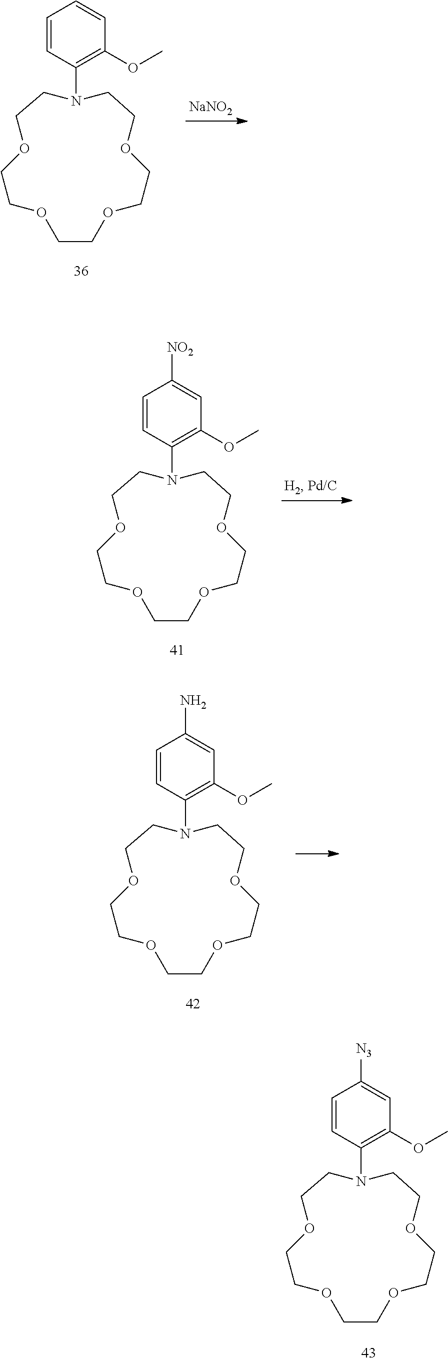

The synthesis of 43 followed the literature according to a modified procedure.²⁵

The appropriate aminocrownether (42) (807 mg, 2.37 mmol) was dissolved in 18 ml 4 M HCl and cooled. At 0° C. was slowly added a solution of 163.6 mg (2.37 mmol) sodium nitrite in 10 ml water. The solution was stirred for 10 minutes at 0° C. Then was added a solution of 230 mg (3.53 mmol) sodium azide in 10 ml water. The resulting solution was stirred for further 10 min at 0° C. and led warm up to room temperature. It was stirred overnight. The mixture was brought to pH 7 with potassium carbonate and extracted 3 times with CHCl₃. The combined organic layers were dried with MgSO₄ and concentrated in vacuum. The residue was purified by column chromatography on silica with CHCl₃/CH₃OH (95/5) as eluent. The product was obtained as a darkbrown oil (265 mg, 31%).

¹H NMR (300 MHz, CDCl₃): δ=3.39-3.4 (m, 4H), 3.60-3.68 (m, 16H), 3.79 (s, 3H)

¹³C NMR (75 MHz, CDCl₃): δ=53.13, 55.47, 70.08, 70.37, 70.48, 70.93, 103.25, 110.61, 122.16, 133.87, 137.25, 153.89

MS (EI) m/z (%): 366 (12) [M]⁺, 338 (100)[M-N₂]⁺.

IR (ATR, cm⁻¹): 2858 (s), 2102 (s), 1504 (s), 1234 (s), 1105 (s).

### N-n-butyl-4-ethinyl-1,8-naphthalimide (50)

### N-n-butyl-4-bromo-1,8-naphthalimide

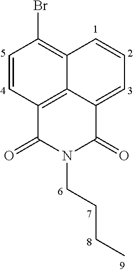 [SMILES: CCCCN1C(=O)c2cccc3c(Br)ccc(c23)C1=O]

The reaction followed the literature according to a modified procedure.²⁶ In a dry 250-ml round bottom flask, under a stream of argon gas, were placed the 4-bromo-1,8-naphthalic anhydride (95 w %, 1.35 g, 4.63 mmol, 1 equiv.) and 1-butylamine (99 w %, 410.35 mg, 5.5 mmol, 1.2 equiv.) in dry ethanol (96 ml) and the reaction mixture was heated at 60° C. for 16 h. After cooling to room temperature, the solid was filtered and washed with 200 ml H₂O to give the product as a light yellow solid.

Yield: 0.800 mg, 52%, mp: 100-102° C. (Lit.: 103-105° C., R_{f}=0.8 (n-hexane/ethyl acetate=4:1 v/v); ¹H NMR, (CDCl₃, 300 MHz) 6 (ppm), J (Hz): 8.63 (d, 1H, J=7.29, H¹), 8.53 (d, 1H, J=8.52, H³), 8.39 (d, 1H, J=7.86, H⁴), 8.01 (d, 1H, J=7.9, H⁵), 7.82 (tr, 1H, J=8.46, H²), 4.16 (t, 2H, J=7.55, H⁶), 1.71 (qu, 2H, J=7.5, H⁷), 1.45 (sx, 2H, J=7.5, H⁸), 0.97 (t, 3H, J=7.32, H⁹); ¹³C NMR, (CDCl₃, 75 MHz) δ (ppm): 163.74, 163.71, 133.30, 132.11, 131.30, 131.21, 130.74, 130.28, 129.12, 128.19, 123.30, 122.45, 40.53, 30.31, 20.52, 13.98;

### N-n-Butyl-4-(trimethylsilylethinyl)-1,8-naphthalimide

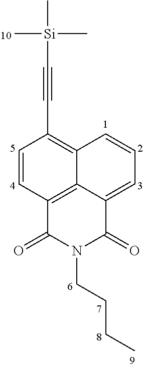 [SMILES: CCCCN1C(=O)c2cccc3c(C#C[Si](C)(C)C)ccc(c23)C1=O]

A dry 100-ml two-necked round-bottom flask was set under argon atmosphere before N-n-Butyl-4-Bromo-1,8-naphthalimide (0.8 g, 2.4 mmol, 1 equiv.), THF (8 mL), Pd(PPh₃)₂Cl₂ (33.8 mg, 0.048 mmol, 0.02 equiv.), PPh₃ (25.3 mg, 0.096 mmol, 0.04 equiv.), CuI (18.3 mg, 0.096 mmol, 0.04 equiv.) and dry TEA (8 ml) were added followed by the trimethylsilylacetylene (331.1 mg, 3.37 mmol, 1.4 equiv.). It was refluxed for 6 h. Then the reaction mixture was allowed to cool down to room temperature and the solvent was evaporated under reduced pressure. The crude product was purified by column chromatorgraphy (50×3 cm, silica gel, CH₂Cl₂=10:1 v/v) to give the productas a yellow solid.

Yield: 0.780 g, 92%, mp: 136.2-138.8° C., R_{f}=0.87; ¹H NMR, (CDCl₃, 300 MHz) 6 (ppm), J (Hz): 8.61 (dd, 2H, H⁴, H³), 8.50 (d, 1H, J=7.62, H¹), 7.88 (d, 1H, J=7.62, H⁵), 7.8 (t, 1H, J=7.62, H²), 4.17 (t, 2H, J=7.5, H⁶), 1.71 (qu, J=7.5, 2H, H⁷), 1.37-1.51 (m, 2H, J=7.53H⁸), 0.97 (t, 3H, J=7.3, H⁹), 0.36 (s, 9H, H¹⁰); ¹³C NMR, (CDCl₃, 75 MHz) δ (ppm): 164.14, 163.86, 132.49, 131.93, 131.68, 131.32, 130.35, 128.07, 127.63, 127.37, 123.11, 122.53, 105.37, 101.44, 40.48, 30.36, 20.54, 13.99, 0.002;

### N-n-Butyl-4-ethinyl-1,8-naphthalimide (50)

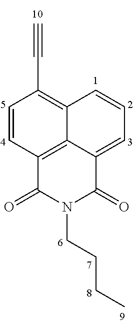 [SMILES: C#Cc1ccc2c3c(cccc13)C(=O)N(CCCC)C2=O]

N-n-Butyl-4-(trimethylsilylethinyl)-1,8-naphthalimide (710 mg, 2.03 mmol) was placed into a 100-ml round-bottom flask and in dry methanol (50 ml) and dry K₂CO₃ (1.09 g, 7.92 mmol, 3.9 equiv.) were added. It was heated for 16 h at room temperature. CH₂Cl₂ (40 ml) was added to the reaction mixture and the organic phase was washed with water (2×20 ml). The combined organic phases were dried over Na₂SO₄ and concentrated in vacuo. The crude product was purified by column chromatography (50×3 cm, silica gel, CH₂Cl₂/ethyl acetate=10:1 V/V) to give the product as a yellow solid. Yield: 0.200 g, 35%, mp.: 127.5-130.8° C., R_{f}=0.81; ¹H NMR, (CDCl₃, 600 MHz) 6 (ppm), J (Hz): 8.65 (d, 1H, J=8.43, H³), 8.63 (d, 1H, J=7.32, H⁴,), 8.53 (d, 1H, J=7.56, H¹), 7.93 (d, 1H, J=7.56, H⁵), 7.82 (t, 1H, J=8.25, H²), 4.17 (t, 2H, J=7.62, H⁶), 3.73 (s, 1H, H¹⁰), 1.71 (qu, 2H, J=7.56, H⁷), 1.44 (sx, 2H, J=7.56, H⁸), 0.97 (t, 3H, J=7.38, H⁹); ¹³C NMR, (CDCl₃, 75 MHz) δ (ppm): 164.05, 163.78, 132.24, 132.12, 131.77, 130.245, 128.09, 127.81, 126.30, 123.24, 123.07, 122.01, 86.52, 80.51, 40.50, 30.36, 20.52, 13.95;

### 6-azido-N-ethyl-1,8-naphthalimide (51) ²⁷

### N-n-ethyl-4-bromo-1,8-naphthalimide

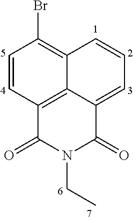 [SMILES: CCN1C(=O)c2cccc3c(Br)ccc(c23)C1=O]

The synthesis followed the literature according to a modified procedure.′^{1b}

A suspension of 4-bromo-1,8-naphthalic anhydride (2 g, 7.2 mmol, 1 equiv.) and ethylamine (70 w % in water,0.69 ml, 8.66 mmol, 1.2 equiv.) in 100 ml 1.4-dioxane was refluxed for 6 h giving a brownish solution. The solution was cooled to room temperature before it was poured onto 300 ml ice water resulting in an off white precipitate. After complete melting of the ice, the precipitate was collected by filtration and was washed with water. The resulting solid was dried in vacuo in a desiccator equipped with CaCl₂ to give the product as an off white solid which was directly used in the next step.

Yield: 1.72 g, 78%, mp: 145-146° C., ¹H NMR, (CDCl₃, 500 MHz) 6 (ppm), J (Hz): 8.62 (dd, 1H, H¹), 8.34 (dd, 1H, H³), 8.37 (d, 1H, J=7.85, H⁴), 8.0 (d, 1H, J=7.80, H⁵), 7.81 (tr, 1H, J=7.35, H²), 4.22 (q, 2H, J=7.15, H⁶), 1.32 (tr, 3H, J=7.10, H⁷); MS (EI): m/z calcd.: 303. found, 303+305: [m/z-CH₂CH₃]⁺ calcd.:275. found 275+277;

### 6-azido-N-ethyl-1,8-naphthalimide (51)

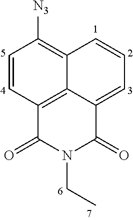 [SMILES: CCN1C(=O)c2cccc3c(N=[N+]=[N-])ccc(c23)C1=O]

The synthesis followed the literature and references therein. In a 25-ml round-bottom flask was placed the N-n-ethyl-4-bromo-1,8-naphthalimide (0.5 g, 1.64 mmol, 1 equiv.) and sodium azide (0.53 g, 8.2 mmol, 5 equiv.). Then N-methylpyrrolidinone (7 ml) was added and the mixture was heated at 110° C. for 1.5 h. The solution was allowed to cool to room temperature and diluted with 25 ml water. It was extracted with ethyl acetate (3×20 ml) and the combined organic phases were washed with brine. The organic phase was dried over MgSO₄ and concentrated under reduced pressure. The crude product was purified by column chromatography (silica gel, hexane/ethyl acetate=4:1 v/v) to give the product as a yellow solid.

Yield: 0.254 g, 58%, ¹H NMR, (CDCl₃, 300 MHz) 6 (ppm), J (Hz): 8.62 (dd, 1H, H¹), 8.56 (dd, 1H, H⁴), 8.41 (dd, 1H, H³), 7.72 (m, 1H, H²), 7.45 (dd, 1H, H⁵), 4.23 (q, 2H, J=7.14, H⁶), 1.32 (tr, 3H, J=7.08, H⁷); ¹³C NMR, (CDCl₃, 75 MHz) δ (ppm): 163.92, 163.51, 143.50, 132.26, 131,74, 129.27, 128.82, 126.96, 124.49, 122.85, 119.15, 114.78, 35.65, 13.48; MS (EI): m/z calcd.:266. found, 266; [m/z-N₂]⁺ calcd.:238. found 238; IR (KBr, cm⁻¹): 2131 (s, N₃).

### 10-ethynylanthracene (52)

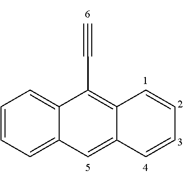 [SMILES: C#Cc1c2ccccc2cc2ccccc12]

The synthesis followed the literature.²⁸

Yield: 78%, ¹H NMR, (CDCl₃, 300 MHz) 6 (ppm)): 8.58 (dd, 2H, H¹), 8.46 (s, 1H, H⁵), 8.02 (dd, 2H, H⁴), 8.62-8.45 (m, 4H, H″), 3.99 (s, 1H, H⁶)

### General Procedure for the Synthesis of Azidoanthracene Derivatives

### 9-(azidomethyl)-anthracene (53)

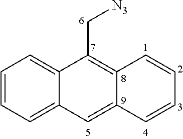 [SMILES: [N-]=[N+]=NCc1c2ccccc2cc2ccccc12]

The synthesis followed the literature according to a modified procedure.²⁹ A suspension of chloromethylanthracene (1 g, 4.41 mmol, 1 equiv.) and sodium azide (0.43 g, 6.62 mmol, 1.5 equiv.) in 30 ml acetonitrile was refluxed for 5 h. After the reaction was complete (monitoring via DC) the reaction mixture was cooled to room temperature and the resulting solid was filtered off. The solution was concentrated in vacuo and the resulting yellow solid was purified by column chromatography on silica gel (n-hexane/ethyl acetate=3:1 v/v).

Yield: 0.92 g (90%), mp.: 78-80° C., ¹H-NMR, (CDCl₃, 500 MHz) 6 (ppm), J (Hz): 8.5 (s, 1H, H⁵), 8.29 (d, 2H, J=8.85, H¹), 8.05 (d, 2H, J=8.40, H⁴), 7.60 (m, 2H, H²), 7.52 (m, 2H, H³), 5.31 (s, 2H, H⁶); ¹³C-NMR, (CDCl₃, 125 MHz) δ (ppm): 131.45, 130.79, 129.39, 129.09, 126.94, 125.87, 125.30, 123.31, 46.42; MS (EI): m/z calcd.:233. found, 233; FT-IR (KBr), cm⁻¹: 2095 (s, —N₃), 1444 (m, —CH₂—).

### 10-(azidomethyl)-9-carbonitrilanthracene (54)

The Synthesis of the 10-Bromomethyl-9-cyanoanthracene via 10-Methyl-9-cyanoanthracene followed the literature.³⁰

### 10-methyl-9-cyanoanthracene

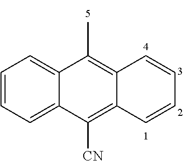 [SMILES: Cc1c2ccccc2c(C#N)c2ccccc12]

The product was collected after crystallization from acetic anhydride in form of yellow needles.

Yield: 81%, mp: 207-208° C.; ¹H-NMR, (CDCl₃, 300 MHz) 6 (ppm), J (Hz): 8.38 (d, 2H, J=8.61, H¹), 8.29 (d, 2H, J=8.79, H⁴), 7.69-7.5 (m, 4H, H^{2,3}), 3.09 (s, 3H, H⁵); ¹³C-NMR, (CDCl₃, 75 MHz) δ (ppm): 138.24, 132.98, 129.51, 128.45, 126.29, 126.17, 125.48, 117.83, 104.41, 14.91; HRMS (⁺ESI): m/z calcd for (M+H)⁺, 218.10. found, 218.15.

### 10-bromomethyl-9-cyanoanthracene

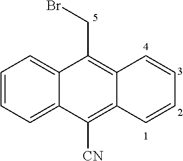 [SMILES: N#Cc1c2ccccc2c(CBr)c2ccccc12]

Recrystallization from n-hexanes/CHCl₃ at −20° C. gave the product as a yellow solid (needles).

Yield: 90,1% mp.: 248-254° C., Rf=0.33 (n-hexanes/ethyl acetate=8:2 v/v);

¹H-NMR, (CDCl₃, 300 MHz) 6 (ppm), J (Hz):8.49-8.43 (m, 2H, H¹), 8.39-8.31 (m, 2H, H⁴), 7.77-7.69 (m, 4H^{2,3}), 5.46 (s, 2H, H⁵); ¹³C-NMR, (CDCl₃, 75 MHz) δ (ppm): 135.07, 133.18, 128.95, 128.85, 127.68, 126.45, 124.42, 117.20, 107.84, 24.97; FT-IR (KBr,cm⁻¹): 3051 (w, Br—CH₂—), 2212 (s, —C≡N), 1444 (m, —CH₂—).

### 10-(azidomethyl)-9-carbonitrilanthracene (54)

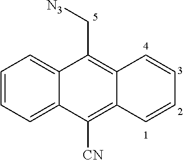 [SMILES: N#Cc1c2ccccc2c(CN=[N+]=[N-])c2ccccc12]

The synthesis followed the procedure of the preparation of 9-(azidomethyl)-anthracene as described above. The crude product was recrystallized from CH₂Cl₂/MeOH to give the product as yellow fluffy needles.

Yield: 39%, R_{f}=0.41; ¹H-NMR, (CDCl₃, 300 MHz) 6 (ppm), J (Hz): 8.48 (d, 2H, J=8.84, H¹), 8.35 (d, 2H, J=8.13, H⁴), 7.79-7.65 (m, 4H, H^{2,3}), 5.33 (s, 2H, H⁵); ¹³C-NMR, (CDCl₃, 75 MHz) δ (ppm): 133.04, 132.90, 129.94, 128.85, 127.84, 126.52, 124.48, 117.07, 108.08, 46.29; HRMS (⁺ESI): m/z calcd for (M+H)⁺, 259.10. found, 259.15; FT-IR (KBr) u (cm⁻¹): 2213 (s, —C≡N), 2100 (s, —N₃), 1444 (m, —CH₂—).

### 9-(azidomethyl)-10-methylanthracene (55)

### 9-(10-methyl)-bromomethyl anthracene³¹

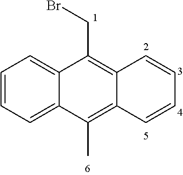 [SMILES: Cc1c2ccccc2c(CBr)c2ccccc12]

A 500-ml round-bottom flask, equipped with condenser, thermometer and stirbar was set under argon atmosphere and the anthraquinone (10.5 g, 0.050 mol) and dry THF (375 ml) were added. The mixture was cooled to −78° C. before a solution of methyl lithium (110 ml, 1.4 M in diethylether, 0.151 mol, 3.05 equiv.) was added cautiously via a syringe. The reaction mixture was slowly allowed to warm up to room temperature and was stirred at this temperature for 2 h before water (100 ml) was added. The mixture was extracted with CH₂Cl₂ (2×100 ml) and the combined organic phases were concentrated to give the 9,10-dihydroxy-9,10-dimethylanthracene as a white solid which was used in the next step without further purification. It was dissolved in THF (250 ml) and a solution of THF (130 ml) and HBr (150 ml,48 w %) was added drop wise. The reaction mixture was stirred at ambient temperature for 30 min where upon yellow crystals formed. The crystals were collected by filtration, washed with water and dried under vacuum to give the final product.

Yield: 10.16 g, 72%, mp: 177.5-179.0° C., R_{f}=0.25 (CHCl₃/MeOH=97:3 v/v); ¹H-NMR, (CDCl₃, 300 MHz) 6 (ppm), J (Hz): 8.35 (t, 4H, J=7.56, H^{1,5}), 7.65 (tr, 2H, J=6.9, J=8.2, H³), 7.54 (tr, 2H, J=6.9, J=8.2, H⁴), 5.55 (s, 2H, H¹), 3.09 (s, 3H, H⁶); ¹³C-NMR, (CDCl₃, 75 MHz) δ (ppm): 133.30, 130.28, 129.58, 126.39, 126.32, 125.67, 125.39, 124.26, 27.97, 14.74; HRMS (⁺ESI): m/z calcd for (M+H)⁺, 286.19. found, 286.32;

### 9-(azidomethyl)-10-methylanthracene (55)

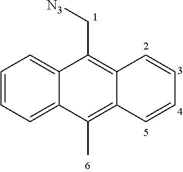 [SMILES: Cc1c2ccccc2c(CN=[N+]=[N-])c2ccccc12]

The synthesis followed the procedure of the preparation of 9-(azidomethyl)-anthracene as described above.

Yield: 90%, R_{f}=0.32; ¹H-NMR, (CDCl₃, 300 MHz) 6 (ppm), J (Hz): 8.39-8.31 (m, 4H, H^{2,5}), 7.63-7.53 (m, 4H, H^{3, 4}), 5.34 (5, 2H, H¹), 3.14 (5, 3H, H⁶); ¹³C-NMR, (CDCl₃, 75 MHz) δ (ppm): 132.91, 130.62, 130.03, 126.44, 125.74, 125.22, 124.29, 46.69, 14.64; MS (EI): m/z: 247; [m/z-.N₂]=218; [m/z-.CH₂N₃]=205; FT-IR (KBr), cm⁻¹: 2214 (s, C≡N), 2114 (5, N₃), 1444 (m, CH₂).

### N,N-bis(2-hydroxyethyl)-2-methoxyethyloxyaniline

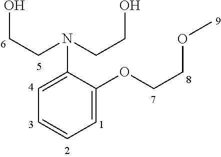 [SMILES: COCCOc1ccccc1N(CCO)CCO]

The Synthesis followed the literature according to a modified procedure.³² A mixture of N-(2-methoxyethoxy)-2-nitrobenzole³³ (21.9 g; 0.131 mol; 1 equiv.). 2-chlorethanol (52.77 g; 0.656 mol; 5 equiv.) and CaCO₃ (18.37; 0.184 mol; 1.4 equiv.) in dist. water (300 ml) was stirred for 6 days at 60° C. After cooling to room temperature, Na₂CO₃ (75.0 g; 0.7 mol; 5.34 equiv.) was added and it was stirred for 40 Min at 60° C. after which the solid is filtered off. The aqueous phase is extracted tertbutyl-methyl-ether (3×500 ml). The combined organic phases are dried over MgSO₄ and concentration in vacuo gave the crude product as a brownish oil, which was purified by column chromatography (silica gel. ethyl acetate). Yield: (30.1 g. 90%).

¹H NMR (CDCl₃. 300 MHz): δ=3.15 (tr. 4H. H⁶). 3.43 (s. 3H. H⁹). 3.47 (tr. 4H. H⁵). 3.74 (tr. 2H. H⁸). 4.11 (tr. 2H. H⁷). 6.91 (dd. 1H. H⁴). 6.98 (tr. 1H. H²). 7.11 (tr. 1H. H³). 7.22 (d. 1H. H¹); ¹³C NMR (CDCl₃. 75 MHz): δ=57.93; 58.93; 59.66; 67.80; 70.70; 113.33; 122.23; 125.51; 126.01; 139.28; 155.25; HRMS (⁺ESI): m/z calc. for (M+H)⁺. 256.15. found. 256.13.

### 2-methoxyethoxyphenylaza-18-crown-6-ether

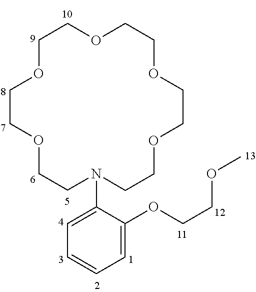 [SMILES: COCCOc1ccccc1N1CCOCCOCCOCCOCCOCC1]

The synthesis followed the literature according to a modified procedure.³⁴ The N,N-bis(2-hydroxyethyl)-2-methoxyethyloxyaniline (15.46 g; 60.55 mmol; 1 equiv.) was dissolved in 440 ml dry acetonitrile and set under argon atmosphere. Under a stream of argon sodiumhydride (80% ig; 4.5 g; 2.86 equiv.) was added to the reaction mixture over a period of 1 h. The 1,17-ditosyl-3,6,9,12,15-pentaoxaheptadecane (30.4 g; 60.55 mmol; 1 equiv.) was dissolved in 216 ml dry acetonitrile and added dropwise to the refluxing reaction mixture over 4 h. It was heated to reflux for 11 h before the yellow suspension was allowed to cool to room temperature. It was filtered and the solvent was removed to give a brown oil. which was purified by column chromatography (silica gel. CHCl₃. MeOH, 95/5, v/v) to give the product as a light brown oil.

¹H NMR (CDCl₃. 300 MHz): δ=3.41 (s. 3H. H¹³). 3.45-3.66 (m. 24H. H⁵⁻¹⁰). 3.73 (tr. 2H. J=4.9 Hz. H¹²). 4.10 (tr. 2H. J=4.9 Hz. H¹³). 6.82-7.12 (m. 4H. H¹⁻⁴); ¹³C NMR (CDCl₃. 75 MHz): δ=56.70; 59.91; 59.83; 67.83; 69.57-71.18; 114.39; 121.24; 122.21; 124.21; 151.71; 153.9; HRMS (⁺ESI): m/z ber. für (M+H)⁺. 255.16; gefunden. 255.13.

### N-2-methoxyethoxy-4-nitrophenylaza-18-crown-6-ether

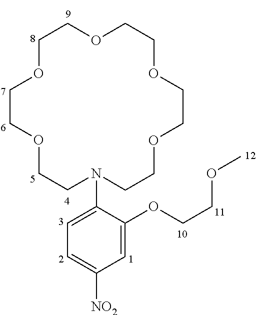 [SMILES: COCCOc1cc([N+](=O)[O-])ccc1N1CCOCCOCCOCCOCCOCC1]

The synthesis followed the literature according to a modified procedure.³⁵ The 2-methoxyethoxyphenylaza-18-crown-6-ether (4.4 g; 10.65 mmol; 1 equiv.) was dissolved in a mixture of dist. water (340 ml) and glacial acetic acid (34 ml) before a solution of NaNO₂ (0.81; 11.7 mmol; 1.1 equiv.) in deionized water was added drop wise over a period of 10 min. It was stirred for 16 h at room temperature. The deep orange reaction mixture was neutralized with LiOH followed by extraction with CH₂Cl₂ (3×100 ml) The combined organic phases were dried over MgSO₄ and the solvent was removed in vacuo to give the crude product as an orange oil. Purification by column chromatography yielded the product as an orange oil (silica gel. CH₃Cl/MeOH, 95/5, v/v) (1.1 g. 22.4%).

¹H NMR (CDCl₃. 300 MHz): δ=3.40 (s. 3H. H¹²). 3.60-3.70 (m. 24H. H⁴⁻⁹). 3.73 (m, 2H, H¹¹). 4.14 (m, 2H, H¹⁰). 6.88 (d. 1H. J=9.04 Hz. H³). 7.65 (d, 1H. J=2.5 Hz, H¹). 7.80 (dd, 1H, H²); HRMS (⁺ESI): m/z calc. (M+H)⁺. 459.23. found, 459.26. ¹³C NMR (CDCl₃. 75 MHz): δ=52.83, 58.82, 67.91, 69.93, 70.52, 70.55, 72.57, 70.64, 70.69, 70.77, 108.20. 115.65, 118.57, 139.07, 146.32, 148.38; HRMS (⁺ESI): m/z calc. for (M+H)⁺. 459.23. found. 459.25.

### N-3-(-2-methoxyethoxyphenylaza-18-crown-6-)aniline

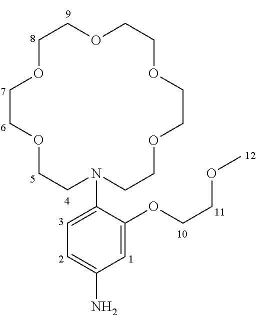 [SMILES: COCCOc1cc(N)ccc1N1CCOCCOCCOCCOCCOCC1]

The N-2-methoxyethoxy-4-nitrophenylaza-18-crown-6-ether (0.22 g. 0.48 mmol) was dissolved in dry methanol (30 ml) After addition of Pd/C (30 mg) it was hydrated in an autoclave for 16 h at 75 bar. The catalyst was filtered of through a bed of Celite® and the solvent was removed in vacuo to yield a colorless oil (0.2 g; 97%). Note that the compound decomposes quickly when exposed to air, resulting in a purple colour. That's why it was used in the next step without further purification.

HRMS (⁺ESI): m/z calc. for (M+H)⁺. 429.26. found. 429.20.

### N-4-(azido)-2-methoxyethoxyphenylaza-18-crown-6-ether (56)

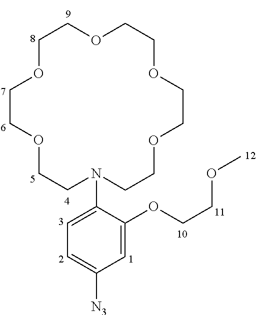 [SMILES: COCCOc1cc(N=[N+]=[N-])ccc1N1CCOCCOCCOCCOCCOCC1]

Due to the instability of the N-3-(-2-methoxyethoxyphenylaza-18-crown-6-)aniline. the reaction was carried out under argon atmosphere and in oven dried glassware. The aqueous solutions were degassed and flushed with argon prior use.

The N-3-(-2-methoxyethoxyphenylaza-18-crown-6-)aniline (0.2 g;0.467 mmol; 1 equiv.) was dissolved in HCl (3.6 ml; 4M) gelost and cooled to 0° C. before a solution of NaNO₂ (32 mg; 0.467 mmol; 1 equiv.) in 1.8 ml H₂O was slowly added via a syringe. The reaction mixture was stirred at this temperature for 10 min. before a solution of NaN₃ (0.45 mg. 0.7 mmol; 1.5 equiv.) in 1.8 ml H₂0 was added dropwise followed by 10 in of stirring at 0° C. The solution was allowed to warm up to room temperatur and stirred at ambient temperatuer for 14 h. The reaction mixture was neutralized with Li₂CO₃ before it was extracted with 3×50 ml CHCl₃. The organic layers were combined, dried with MgSO₄, filtered and the solvent was removed under reduced pressure. The crude product was purified by column chromatography (silica gel. CH₃Cl/MeOH (95/5) yielding the desired product as light yellow oil (0.13 g. 61.3%).

¹H NMR (CDCl₃. 300 MHz): δ=3.4 (s. 3H. H¹²). 3.52-3.68 (m. 24H. H⁴⁻⁹). 3.75 (tr. 2H. J=4.9 Hz. H¹¹). 4.10 (tr. 2H. J=4.9 Hz. H¹⁰). 6.51 (s. 1H. H³). 6.60 (d. 1H. J=8.48. H²). 7.09 (d. 1H. J=8.48 Hz. H²); ¹³C NMR (CDCl₃. 75 MHz): δ=53.18. 59.06. 67.82. 69.76. 70.31. 70.55. 70.72. 70.61. 71.00. 105.02. 111.48. 123.52. 134.51. 136.95. 153.87; HRMS (⁺ESI): m/z calc. for (M+H)⁺found. 455.17.

IR (ATR. cm⁻¹): 2106 (strong. —N₃)

### 6-ethynyl-2-hexadecyl-1.8-naphthalimid (57)

The synthesis of the 6-ethynyl-2-hexyl-1.8-naphthalimidfollowed the literature according to a modified procedure.³⁶

### 6-bromo-2-hexadecyl-1.8-naphthalimid

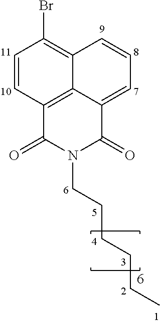

The 4-bromonaphthalene anhydride (1.0 g. 3.61 mmol. 1 Eq.) and n-butylamine (1.04 g. 4.32 mmol. 1.2 Äq.) were stirred in dry ethanol (96 ml) at 60 C for 16 h. The reaction mixture was allowed to warm up to room temperature and before the solid was filtered off. It was washed with 200 ml H₂O and dried in high vacuo to give the product as a light yellow solid (1.74 g; 93%)

¹H NMR (CDCl₃. 300 MHz): δ=0.89 (tr. 3H. H¹). 1.25-1.48 (m. 24H. H²⁻⁴). 1.73 (qu. 2H. H⁵). 4.17 (tr. 2H. H⁶). 7.85 (tr. 1H. J=7.35 Hz. H⁸). 8.06 (d. 1H. J=7.72 Hz. H¹¹). 8.57 (d. 1H. J=7.91 Hz. H¹⁰). 8.57 (dd. 1H. H⁷). 8.66 (d. 1H. J=7.35 Hz. H⁹); ¹³C NMR (CDCl₃. 75 MHz): δ=14.50. 23.08. 27.52. 28.47. 29.75. 29.95. 30.00. 30.03. 30.05. 30.06. 30.09. 32.32. 41.02. 122.69. 123.55. 128.42. 129.34. 130.50. 130.96. 131.43. 131.52. 132.33. 133.50. 163.90. 163.92; MS (EI): m/z calcd.: 505. found. 499+501;

### 2-hexadecyl-6-((trimethylsilyl)ethynyl)-1.8-naphthalimid

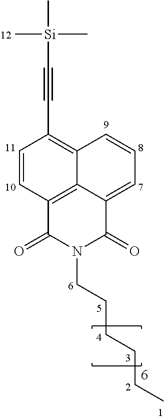

Under argon atmosphere. to a dry flask provided with the 6-bromo-2-hexadecyl-1.8-naphthalimid (0.916 g; 1.83 mmol; 1 equiv.). Pd(PPh₃)₂Cl₂ (26.0 mg. 0.037 mmol. 0.02 equiv.). PPh₃ (19.0 mg. 0.037 mmol. 0.04 equiv.). CuI (14.0 mg. 0.037 mmol. 0.04 equiv.) and dry triethylamine were added. Dry THF (10 mL) was transfered into the flask via a canula followed by the dropwise addition of trimethylsilylacetylene (0.252 mg. 2.56 mmol. 1.4 equiv.). The reaction mixture was heated to reflux for 6 h and then allowed to cool down to room temperature. The solvent was removed and the residue was taken up CHCl₃. Water was added and it was extracted with 3×30 ml CHCl₃. The organic layers were combined. dried over MgSO₄ and filtered. The solvent was removed in vacuo to yield the desired product quantitatively as a grey solid (0.94 g. 100%).

¹H NMR (CDCl₃. 300 MHz): δ=0.35 (s. 9H. J=6.97 Hz. H¹²). 0.86 (tr. 3H. H¹). 1.23-1.34 (m. 24H. H²⁻⁴). 1.71 (qu. 2H. J=7.54 Hz. H⁵). 4.14 (tr. 2H. J=7.72 Hz. H⁶). 7.80 (tr. 1H. J=7.35. H⁸). 7.89 (d. 1H. J=7.35 Hz. H⁷). 8.51 (d. 1H. J=7.72 Hz H¹¹). 8.62 (dd. 2H. H^{9,10}); ¹³C NMR (CDCl₃. 75 MHz): δ=9.08. 14.03. 22.64. 27.13. 28.12. 29.31. 29.34. 29.51. 29.56. 29.60. 29.62. 29.63. 29.65. 31.89. 40.57. 46.15. 101.32. 105.20. 122.45. 123.36. 127.46. 130.16. 131.15. 131.48. 132.29. 163.163.94; MS (EI): m/z calcd.: 517. found. 517;

### 6-ethynyl-2-hexadecyl-1.8-naphthalimid (57)

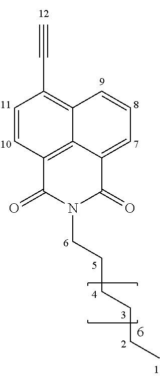

The 2-hexadecyl-6-((trimethylsilyl)ethynyl)-1.8-naphthalimid (198 mg. 0.383 mmol; 1 equiv.) was dissolved in 10 ml dry methanol. dry K₂CO₃(212 mg. 1.53 mmol. 4 equiv.) was added and it was stirred for 50 h at roomtemperature The red brown suspension was filtered and the collected solid was purified by column chromatography (80×3 cm. Kieselgel. Dichlormethan/Essigsaureethylester=95:5 v/v) to give the pure product as a light yellow solid (0.91 g. 53%).

¹H NMR (CDCl₃. 300 MHz): δ=0.87 (tr. 3H. J=6.78 Hz. H¹). 1.24-1.44 (m. 26H. H²⁻⁴). 1.72 (qu. 2H. J=7.53 Hz. H⁵). 3.73 (s. 1H. H¹²). 4.19 (tr. 2H. J=7.53 Hz. H⁶). 7.82 (tr. 1H. J=7.35 Hz. H⁸). 7.96 (d. 1H. J=7.72 Hz. H¹¹). 8.26 (d. 1H. J=7.53 Hz. H¹⁰). 8.64 (dd. 2H. H^{7,9}); ¹³C NMR (CDCl₃. 75 MHz): δ=14.22. 22.83. 27.32. 28.30. 29.50. 29.52. 29.70. 29.56. 28.80. 29.82. 29.85. 32.08. 40.79. 80.56. 86.50. 123.16. 123.30. 126.31. 127.80. 128.14. 130.24. 131.76. 131.79. 132.16. 132.23. 163.76. 164.03; MS (EI): m/z calcd.: 445. found. 445;

### General Procedure of the CuAAC Reaction

The Cu(I) catalyzed reaction between an azide and an alkyne (CuAAC) has been performed as follows:

All reactions were performed on a mmol-scale. To a solution of azide (1 eq) and alkyne (1 eq) in THF/H₂O (3/1) was added CuI (5 mol %) and sodium ascorbate (2.5 mol %). It was stirred overnight at 50° C.

The THF was removed under reduced pressure and the residue was taken up in CHCl₃. It was washed with water and the organic phase was dried over MgSO₄. The organic phase was concentrated and the residue was chromatographed on silica gel eluting with CH₂Cl₂/MeOH (95/5).

### Example 1

### Synthesis of 3-{4-[4-(1,4,7,10,13-pentaoxa-16-azacyclooctadecan-16-yl)phenyl]-1H-1,2,3-triazol-1-yl}-7-(diethylamino)-2H-chromen-2-one (1)

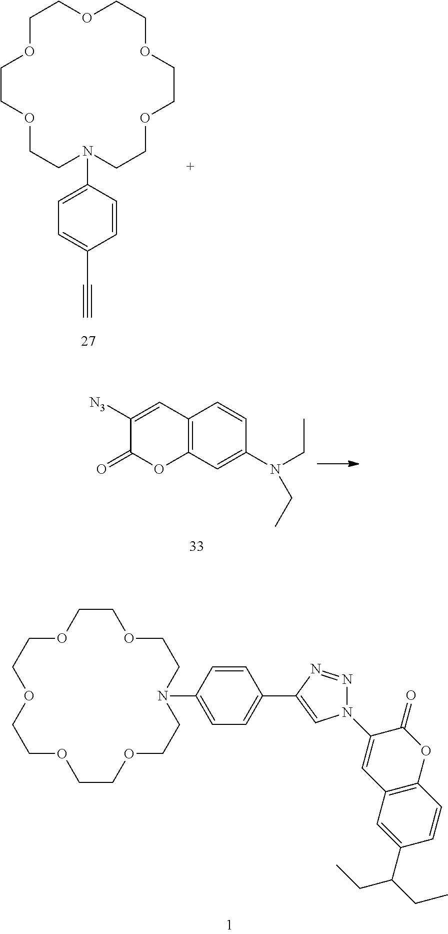 [SMILES: C#Cc1ccc(N2CCOCCOCCOCCOCCOCC2)cc1 ; CCN(CC)c1ccc2cc(N=[N+]=[N-])c(=O)oc2c1 ; CCC(CC)c1ccc2oc(=O)c(-n3cc(-c4ccc(N5CCOCCOCCOCCOCCOCC5)cc4)nn3)cc2c1]

The synthesis followed the general procedure of the CuAAC reaction. Yield: 49%, ¹H NMR (CDCl₃, 500 MHz): δ=1.23 (t, 6H, J=7.25 Hz), 3.44 (q, 4H, J=7.25 Hz,), 3.64-3.67 (m, 20H), 3.72 (t, 4H, J=5.68 Hz), 6.55 (s, 1H), 6.66 (dd, 1H), 6.75 (d, 2H, J=8.52 Hz), 7.41 (d, 1H, J=8.83 Hz), 7.74 (d, 2H, J=8.83 Hz), 8.15 (s, 1H), 8.38 (s, 1H); ¹³C (CDCl₃, 75 MHz): δ=13.53, 46.05, 52.38, 69.78, 71.89, 98.08, 108.27, 110.91, 112.67, 118.12, 119.22, 119.70, 127.96, 130.95, 135.34, 148.82, 149.06, 152.40, 156.79, 158.02; HRMS (⁺ESI): m/z calcd. for (M+H)⁺, 622.32. found, 623.33; UV/Vis (Acetonitrile), λₘₐₓ (ε)=410 nm (21228 M⁻¹ cm⁻¹), λₘₐₓ (ε)=288 nm (20211 M⁻¹ cm⁻¹).

### Example 2

### Synthesis of 3-{1-[4-(1,4,7,10,13-pentaoxa-16-azacyclooctadecan-16-yl)phenyl]-1H-1,2,3-triazol-4-yl}-7-(diethylamino)-2H-chromen-2-one (2)

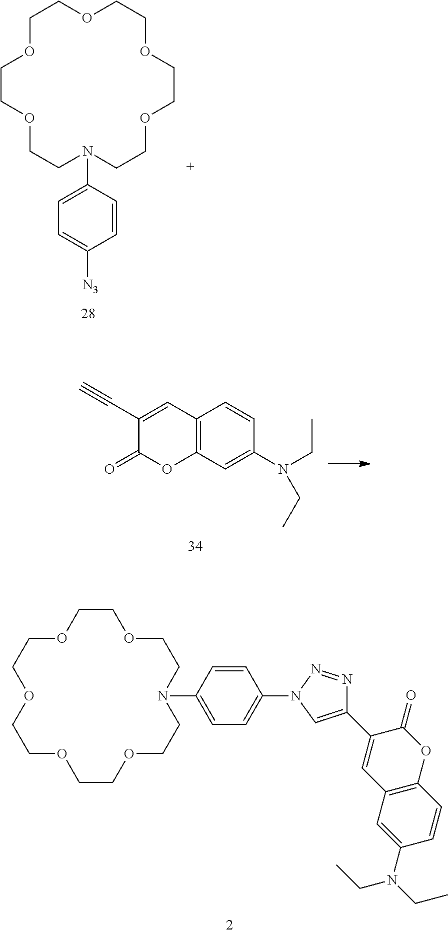 [SMILES: [N-]=[N+]=Nc1ccc(N2CCOCCOCCOCCOCCOCC2)cc1 ; C#Cc1cc2ccc(N(CC)CC)cc2oc1=O ; CCN(CC)c1ccc2oc(=O)c(-c3cn(-c4ccc(N5CCOCCOCCOCCOCCOCC5)cc4)nn3)cc2c1]

The synthesis followed the general procedure of the CuAAC reaction.

Yield: 55%, ¹H NMR (CDCl₃, 600 MHz): δ=1.23 (t, 6H, J=7.25 Hz), 3.44 (q, 4H, J=7.25 Hz,), 3.63-3.67 (m, 20H), 3.72 (t, 4H, J=5.68 Hz), 6.53 (s, 1H), 6.62 (dd, 1H), 6.76 (d, 2H, J=8.48 Hz), 7.41 (d, 1H, J=8.85 Hz), 7.76 (d, 2H, J=8.48 Hz), 8.60 (s, 1H), 8.64 (s, 1H); ¹³C (CDCl₃, 150 MHz): δ=12.54, 44.94, 51.51, 68.58, 70.84, 97.13, 108.83, 109.44, 110.87, 111.93, 120.54, 122.12, 126.56, 129.60, 138.51, 142.06, 148.26, 150.82, 156.11, 160.79; HRMS (⁺ESI): m/z calcd. for (M+H)⁺, 622.32. found, 623.35; UV/Vis (Acetonitrile), λₘₐₓ (ε)=413 nm (40057 M⁻¹ cm⁻¹), λₘₐₓ (ε)=293 nm (18136 M⁻¹ cm⁻¹).

### Example 3

### Reference Compound

### Synthesis of N,N-Diethyl-4-[1-(7-diethylaminocoumarin-3-yl)-1H-1,2,3-triazol-4-yl)]aniline (3)

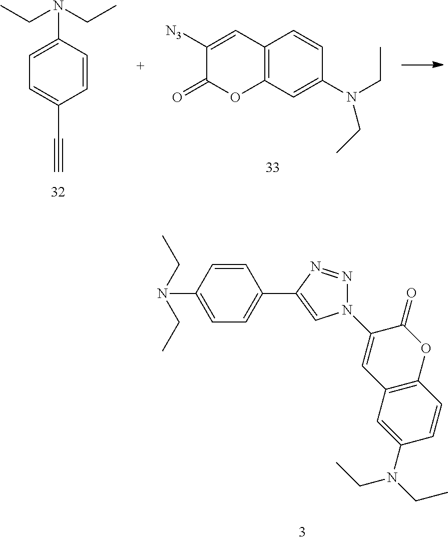 [SMILES: C#Cc1ccc(N(CC)CC)cc1 ; CCN(CC)c1ccc2cc(N=[N+]=[N-])c(=O)oc2c1 ; CCN(CC)c1ccc(-c2cn(-c3cc4cc(N(CC)CC)ccc4oc3=O)nn2)cc1]

The synthesis followed the general procedure of the CuAAC reaction. Yield: 60%, ¹H NMR (CDCl₃, 300 MHz): δ=1.17-1.27 (m, 12H), 3.37-3.50 (m, 8H), 6.57 (s, 1H), 6.68 (dd, 1H), 6.75 (d, 2H, J=8.48 Hz), 7.42 (d, 1H, J=8.85 Hz), 7.76 (d, 2H, J=8.1 Hz), 8.44 (s, 1H), 8.65 (s, 1H), ¹³C (CDCl₃, 75 MHz): δ=12.43, 12.62, 29.69, 44.39, 44.98, 97.08, 107.28, 109.99, 111.80, 117.31, 117.57, 118.61, 127.06, 129.91, 134.15, 147.75, 148.25, 151.41, 155.72, 157.01; HRMS (⁺ESI): m/z calcd. for (M+H)⁺, 431.23. found, 432.32; UV/Vis (acetonitrile), λₘₐₓ (ε)=410 nm (29501 M⁻¹ cm⁻¹), 248 nm (22823 M⁻¹ cm⁻¹).

### Example 4

### Reference Compound

### Synthesis of 3-(4-((1,4,7,10,13-pentaoxa-16-azacyclooctadecan-16-yl)methyl)-1H-1,2,3-triazol-1-yl)-7-(diethylamino)-2H-chromen-2-one (4)

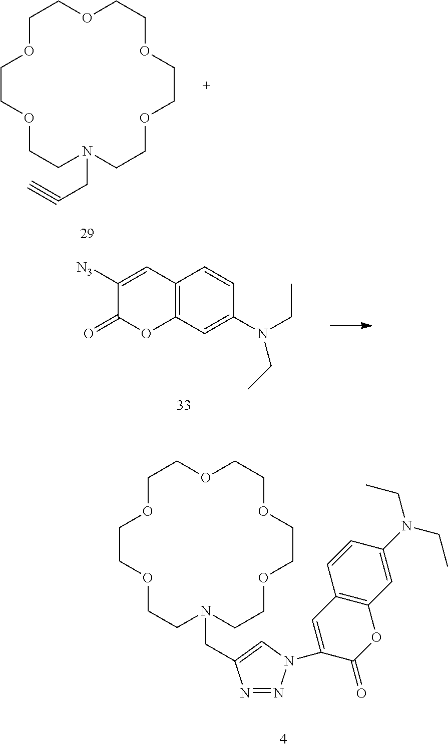 [SMILES: C#CCN1CCOCCOCCOCCOCCOCC1 ; CCN(CC)c1ccc2cc(N=[N+]=[N-])c(=O)oc2c1 ; CCN(CC)c1ccc2cc(-n3cc(CN4CCOCCOCCOCCOCCOCC4)nn3)c(=O)oc2c1]

The synthesis followed the general procedure of the CuAAC reaction.

Yield: 23%, ¹H NMR (CDCl₃, 300 MHz): δ=1.14 (t, 6H, J=7.16 Hz), 2.70 (m, 4H), 3.36 (q, 4H, J=7.16 Hz,), 3.54-3.68 (m, 20H), 3.79 (s, 2H), 6.42 (s, 1H), 6.60 (dd, 1H), 7.40 (d, 1H, J=9.04 Hz), 8.24 (s, 1H), 8.33 (s, 1H); ¹³C (CDCl₃, 75 MHz): δ=12.76, 45.34, 49.51, 53.78, 67.91, 69.72, 97.16, 107.21, 110.60, 124.15, 129.08, 130.67, 131.26, 136.14, 152.12, 156.27, 127.46; HRMS (⁺ESI): m/z calcd. for (M+H)⁺,560.31. found, 560.43; UV/Vis (acetonitrile), λₘₐₓ (ε)=408 nm (10534 M⁻¹ cm⁻¹), 246 nm (11149 M⁻¹ cm⁻¹).

### Example 5

### Synthesis of 7-(diethylamino)-3-(4-(3-methoxy-4-(1,4,7,10-tetraoxa-13-azacyclopentadecan-13-yl)phenyl)-1H-1,2,3-triazol-1-yl)-2H-chromen-2-one (14)

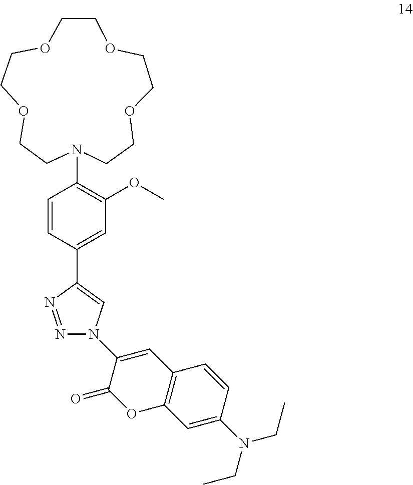 [SMILES: CCN(CC)c1ccc2cc(-n3cc(-c4ccc(N5CCOCCOCCOCCOCC5)c(OC)c4)nn3)c(=O)oc2c1]

A mixture of compound 40 (130 mg, 0.37 mmol), 3-azido-7-diethylaminocoumarin 60 (96.1 mg, 0.37 mmol), copper sulfate pentahydrate (4.6 mg, 5 mol %) and sodium ascorbate (7.3 mg, 10 mol %) in 6 ml THF/water (2/1) was stirred at 60° C. for 48 hours. To the reaction mixture was added water (5 mL) and it was extracted 3 times with CHCl₃(3×10 mL). The combined organic layers were dried with MgSO₄ and concentrated in vacuum. The residue was purified by column chromatography on silica with CHCl₃/CH₃OH (95/5) as eluent. The product was obtained as a dark yellow oil, which crystallised upon standing in a freezer. (90 mg, 40%).

¹H NMR (500 MHz, CDCl₃): δ=1.24 (t, 6H, ³J=7.09 Hz, 1-H), 3.45 (q, 4H, ³J=7.09 Hz, 2-H), 3.51-3.55 (m, 4H, 21-H), 3.64-3.70 (m, 16H, 22-H, 23-H, 24-H, 25-H), 3.93 (s, 3H, 20-H), 6.56 (d, 1H, ⁴J=2.21 Hz, 3-H), 6.68 (dd, 1H, ³J=8.83 Hz, ⁴J=2.36 Hz, 5-H), 7.17 (d, 1H, ³J=7.09 Hz, 16-H), 7.38 (d, 1H, ³J=7.88 Hz, 15-H), 7.43 (d, 1H, ³J=8.99 Hz, 6-H), 7.46 (s, 1H, 19-H), 8.44 (s, 1H, 9-H), 8.74 ppm (s, 1H, 12-H); ¹³C NMR (125 MHz, CDCl₃): δ=12.39 (C1), 44.96 (C2), 53.05 (C21), 55.63 (C20), 70.08, 70.37, 70.49, 70.90 (C22, C23, C24, C25), 97.00 (C3), 107.13 (C7), 109.26 (C19), 110.05 (C5), 116.98 (C10), 118.38 (C15), 119.63 (C12), 120.52 (C16), 124.07 (C18), 129.96 (C6), 134.34 (C9), 139.92 (C13), 147.68 (C14), 151.50 (C4), 152.66 (C17), 155.74 (C8), 156.95 ppm (C11)

ESI-MS: m/z calcd. for [M+H]⁺608.31. found 608.38;

IR (KBr, cm⁻¹): 1130 (s), 1239 (s), 1602 (s),1728 (s), 2855 (s), 2923 (s)

UV/Vis (CH₃CN): λₘₐₓ (ε)=267 (4352), 413 nm (8649).

### Example 6

### Synthesis of 7-(diethylamino)-3-(1-(3-methoxy-4-(1,4,7,10-tetraoxa-13-azacyclopentadecan-13-yl)phenyl)-1H-1,2,3-triazol-4-yl)-2H-chromen-2-one (15)

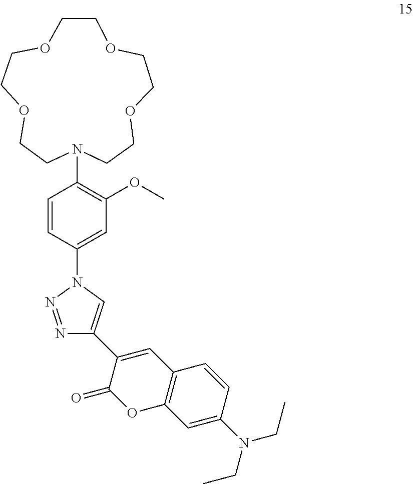 [SMILES: CCN(CC)c1ccc2cc(-c3cn(-c4ccc(N5CCOCCOCCOCCOCC5)c(OC)c4)nn3)c(=O)oc2c1]

A mixture of compound 43 (117.5 mg, 0.32 mmol), 3-acetylen-7-diethylaminocoumarin 61 (77.4 mg, 0.32 mmol), Cu/C (16 mg, 20 mol %) and Triethylamin (32.5 mg, 0.32 mmol) was stirred in THF (4 mL) for 48 hours. Cu/C was filtered off through Celite, washed several times with CHCl₃ and the solvent was removed under reduced pressure. The residue was purified by column chromatography on silica with CHCl₃/CH₃OH (95/5) as eluent. The product was obtained as a dark yellow oil, which crystallised upon standing in a freezer. (91 mg, 47%).

¹H NMR (300 MHz, CDCl₃): δ=1.22 (t, 6H, ³J=7.06 Hz, 1-H), 3.43 (q, 4H, ³J=7.06 Hz, 2-H), 3.51-3.55 (m, 4H, 21-H), 3.65-3.69 (m, 16H, 22-H, 23-H, 24-H,25-H), 3.91 (s, 3H, 20-H), 6.54 (d, 1H, ⁴J=1.98 Hz, 3-H), 6.63 (dd, 1H, ³J=8.76 Hz, ⁴J=2.31 Hz, 5-H), 7.13-7.23 (m, 2H, 15-H, 16-H), 7.32 (s, 1H, 19-H), 7.41 (d, 1H, ³J=8.85 Hz, 6-H), 8.65 (s, 1H, 9-H), 8.66 ppm (s, 1H, 13-H); ¹³C NMR (75 MHz, CDCl₃): δ=12.42 (C1), 44.82 (C2), 53.03 (C21), 55.78 (C20), 70.03, 70.42, 70.94, (C22, C23, C24, C25), 97.03 (C3), 104.73 (C19), 108.67 (C7), 109.32 (C5), 110.59 (C14), 112.45 (C16), 120.40 (C15), 122.05 (C13), 129.49 (C6), 131.15 (C10), 138.49 (C9), 140.25 (C12), 142.14 (C18), 150.78 (C4), 152.83 (C17), 156.00 (C8), 160.62 ppm (C11)

ESI-MS: m/z calcd. for [M+H]⁺608.31. found 608.35;

IR (KBr, cm⁻¹): 1130 (s), 1230 (s), 1600 (s), 1694 (s), 1720 (s), 2861 (s), 2969 (s)

UV/Vis (CH₃CN): λₘₐₓ (ε)=259 (14131), 410 nm (34604)

Elemental analysis (%) calcd. for C₃₂H₄₁N₅O₇ (607.70): C, 63.25, H, 6.80, N, 11.52. found C, 62.91; H, 6.69; N, 11.12.

### Example 7

### Synthesis of 6-(1-(4-(1,4,7,10,13-pentaoxa-16-azacyclooctadecan-16-yl)phenyl)-1H-1,2,3-triazol-4-yl)-2-butyl-1H-benzo[de]isoquinoline-1,3(2H)-dione (11)

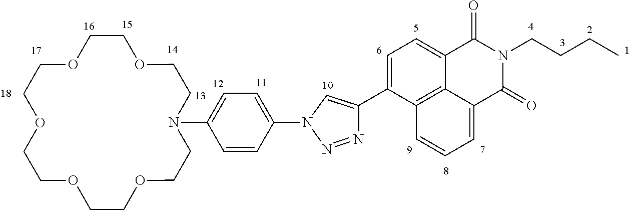 [SMILES: CCCCN1C(=O)c2cccc3c(-c4cn(-c5ccc(N6CCOCCOCCOCCOCCOCC6)cc5)nn4)ccc(c23)C1=O]

The synthesis followed the general procedure of the CuAAC reaction using precursors 28 and 50.

The crude product was purified by column chromatography (65×2 cm, silica gel, CHCl₃/MeOH=98:2 v/v) to yield as a yellow solid.

Yield: 0.086 g, 49%, mp: 136.1-139.0° C., R_{f}=0.52; ¹H NMR, (CDCl₃, 600 MHz) 6 (ppm), J (Hz): 9.1 (dd, 1H, H⁹), 8.63 (dd, 1H, H⁷), 8.61 (d, 1H, J=8.55, H⁵), 8.26 (s, 1H, H¹⁰), 7.99 (d, 1H, J=7.56, H⁶), 7.79 (dd, 1H, H⁸), 7.61, (d, 2H, J=9.12, H¹¹), 6.81 (d, 2H, J=9.12, H¹²), 4.18 (t, 2H, J=7.62, H⁴), 3.74-3.64 (m, 24H, H¹³⁻¹⁸), 1.76-1.69 (m, 2H, H³), 1.45 (sx, 2H, J=7.62, H²), 0.98 (t, 3H, J=7.38, H¹); ¹³C NMR, (CDCl₃, 150 MHz) δ (ppm): 164.34, 164.06, 148.68, 146.15, 145.66, 134.32, 132.92, 131.52, 130.81, 129.40, 128.95, 127.48, 127.35, 125.96, 122.94, 122.56, 121.52, 120.06, 112.96, 111.99, 70.98-70.82, 68.8, 68.60, 51.59, 40.41, 30.32, 20.52, 13.99; HRMS (⁺ESI): m/z calcd for (M+H)⁺, 658.32. found, 658.35;

### Example 8

### Synthesis of 6-(4-(4-(1,4,7,10,13-pentaoxa-16-azacyclooctadecan-16-yl)phenyl)-1H-1,2,3-triazol-1-yl)-2-ethyl-1H-benzo[de]isoquinoline-1,3(2H)-dione (10)

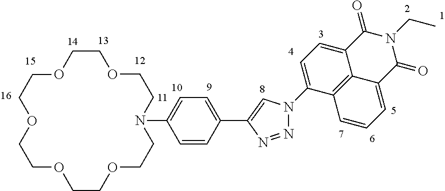 [SMILES: CCN1C(=O)c2cccc3c(-n4cc(-c5ccc(N6CCOCCOCCOCCOCCOCC6)cc5)nn4)ccc(c23)C1=O]

The synthesis followed the general procedure of the CuAAC reaction using precursors 27 and 51.

The crude product was purified by column chromatography (65×2 cm, silica gel, CHCl₃/MeOH=95:5 v/v) to yield as an orange solid.

Yield: 0.024 g, 19%, mp: 213.6-216.4° C., R_{f}=0.14; ¹H NMR, (CDCl₃, 600 MHz) δ (ppm), J (Hz): 8.71 (t, 2H, J=7.47 H³, H⁵), 8.36 (d, 1H, J=8.1, H⁷), 8.09 (s, 1H, H⁸), 7.89 (d, 1H, J=7.47, H^{a}), 7.83 (tr, 1H, J=5.26, H⁶), 7.79, (d, 2H, J=8.7, H⁹), 6.79 (d, 2H, J=8.82, H¹⁰), 4.28 (q, 2H, J=7.12, H²), 3.74-3.68 (m, 24H, H¹¹⁻¹⁶), 1.36 (t, 3H, J=7.12, H¹); ¹³C NMR, (CDCl₃, 150 MHz) δ (ppm): 163.68, 163.16, 149.13, 148.48, 138.59, 132.29, 130.82, 129.87, 129.29, 128.63, 127.31, 126.71, 123.93, 123.46, 123.19, 120.20, 117.23, 112.01, 71.04-70.93, 68.82, 51.50, 35.93, 13.47; HRMS (⁺ESI): m/z calcd for (M+H)⁺, 630.29. found,630.14

### Example 9

### Synthesis of 16-(4-(4-(anthracen-9-yl)-1H-1,2,3-triazol-1-yl)phenyl)-1,4,7,10,13-pentaoxa-16-azacyclooctadecane (5)

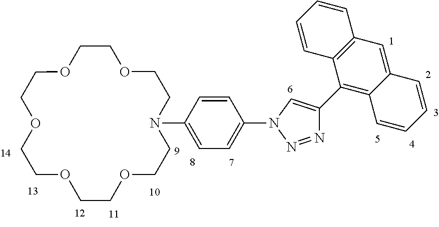 [SMILES: c1ccc2c(-c3cn(-c4ccc(N5CCOCCOCCOCCOCCOCC5)cc4)nn3)c3ccccc3cc2c1]

The synthesis followed the general procedure of the CuAAC reaction using precursors 28 and 52.

The crude product was purified by column chromatography (65×2 cm, silica gel, CHCl₃/MeOH=95:5 v/v) to yield as a light brown oil.

Yield: 23%, ¹H NMR (CDCl₃, 300 MHz): δ=3.53-3.758 (m, 24H, H⁹¹⁴), 6.31 (d, 2H, J=9.231, H⁸), 7.40-7.55 (m, 4H, H^{4,2}), 7.7 (d, 2H, J=9.231, H⁷), 8.03 (m, 4H, H^{5,2}), 8.1 (s, 1H,H⁶), 8.57 (s, 1H, H¹); ¹³C (CDCl₃, 75 MHz): δ=51.16, 68.37, 70.85, 111.24, 119.95, 122.07, 124.54, 125.21, 125.48, 126.07, 126.89, 128.64, 131.02, 131.34, 145.62, 148.02; HRMS (⁺ESI): m/z calcd. (M+H)⁺, 583.34. found, 583.29; UV/Vis (MeCN,) λₘₐₓ (ε)=388 nm (5596 M⁻¹ cm⁻¹), 420 nm (5782 M⁻¹ cm⁻¹).

### Example 10

### Synthesis of 7-(diethylamino)-3-(1-(3-(2-methoxyethoxy)-4-(1,4,7,10,13-pentaoxa-16-azacyclooctadecan-16-yl)phenyl)-1H-1,2,3-triazol-4-yl)-2H-chromen-2-one (7)

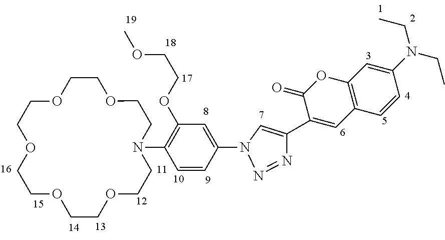 [SMILES: CCN(CC)c1ccc2cc(-c3cn(-c4ccc(N5CCOCCOCCOCCOCCOCC5)c(OCCOC)c4)nn3)c(=O)oc2c1]

The synthesis followed the general procedure of the CuAAC reaction using precursors 56 and 61.

The crude product was purified by column chromatography (65×2 cm, silica gel. CHCl₃/MeOH=95:5 v/v) to yield as a yellow oil.

Yield: 13%, HRMS (⁺ESI): m/z calc. (M+H)⁺. 696.36. found. 696.47.

### Example 11

### Synthesis of 16-(4-(4-(anthracen-9-yl)-1H-1,2,3-triazol-1-yl)-2-(2-methoxyethoxy)phenyl)-1,4,7,10,13-pentaoxa-16-azacyclooctadecane (6)

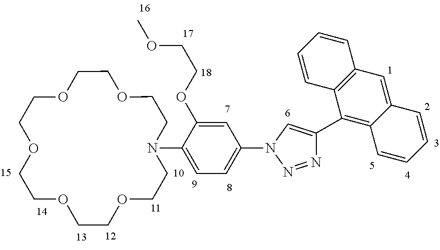 [SMILES: COCCOc1cc(-n2cc(-c3c4ccccc4cc4ccccc34)nn2)ccc1N1CCOCCOCCOCCOCCOCC1]

The synthesis followed the general procedure of the CuAAC reaction using precursors 56 and 52.

The crude product was purified by column chromatography (65×2 cm, silica gel. CHCl₃/MeOH=98:2 v/v) to yield 5 as a light brown oil.

Yield: 19.3%; ¹H NMR (CDCl₃. 600 MHz): δ=3.35-3.75 (m. 27H). 3.81 (tr. 2H). 4.28 (tr. 2H). 7.31 (d. 1H). 7.45 (m. 5H). 7.58 (s. 1H). 7.91 (d. 2H). 8.05 (d. 2H). 8.21 (s. 1H). 8.55 (s. 1H);

### Example 12

### Synthesis of 2-hexadecyl-6-(1-(3-(2-methoxyethoxy)-4-(1,4,7,10,13-pentaoxa-16-azacyclooctadecan-16-yl)phenyl)-1H-1,2,3-triazol-4-yl)-1H-benzo[de]isoquinoline-1,3(2H)-dione (9)

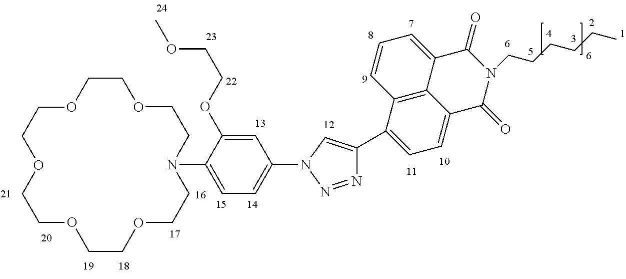

The synthesis followed the general procedure of the CuAAC reaction using precursors 56 and 57.

The crude product was purified by column chromatography (65×2 cm, silica gel. CHCl₃/MeOH=95:5 v/v) to yield 9 as a light brown oil.

Yield: 24%; ¹H NMR (CDCl₃ 600 MHz): δ=0.9 (tr. 3H). 1.27 (m. 24H). 1.39 (qu. 2H). 1.47 (qu. 2H). 1.78 (qu. 2H). 3.40-3.78 (m. 27H). 3.83 (tr. 2H). 4.28 (tr. 2H). 7.21 (d. 1H). 7.35 (d. 1H). 7.50 (s. 1H). 7.86 (tr. 1H). 8.09 (d. 1H). 8.49 (s. 1H). 8.7 (tr. 2H). 9.17 (d. 1H).

### Example 13

### Synthesis of 7-(diethylamino)-3-{4-3-[2-methoxyethoxy)-4{bis(4-methylbenzo[5,6,17,18](O,N,N′,O′)-]-1,7,16, -triaza-cryptand-[3,2,1]-phen-4-yl]-1H-1.2.3-triazol-1yl}-2H-chromen-2-one (13)

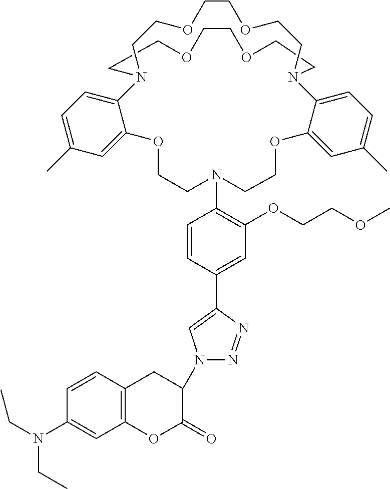 [SMILES: CCN(CC)c1ccc2c(c1)OC(=O)C(n1cc(-c3ccc(N4CCOc5cc(C)ccc5N5CCOCCOCCN(CCOCCOCC5)c5ccc(C)cc5OCC4)c(OCCOC)c3)nn1)C2]

The synthesis followed the general procedure of the CuAAC. Yield: 62%, HRMS (⁺ESI): m/z calc. (M+H)⁺. 976.52. found. 976.59.

### REFERENCES

1 a) E. Tamanini, A. Katewa, L. M. Sedger, M. H. Todd and M. Watkinson, Inorg. Chem, 2009, 48, 319; b) E. Tamanini, K. Flavin, M. Motevalli, M. H. Todd and M. Watkinson, Inorg. Chem., 2010, 49, 3798; c) S. Huang, R. J. Clark, L. Zhu, Org. Lett. 2007, 9, 4999;
2 S. Maisonneuve, Q. Fang, J. Xie, Tetrahedron, 2008, 64, 8716.
3 K. Varazo, F. Xie, D. Gulledge, Q. Wang. Tetrahedron Lett., 2008, 49, 5293.
4 H.-C. Hung, C.-W. Cheng, Y.-Y. Wang, Y.-J. Chen, W.-S. Chung, Eur. J. Org. Chem., 2009, 15, 6360.
5 D. Maity, T. Govindaraju, Inorg. Chem., 2010, 49, 7229.
6 P. D. Jarowski, Y.-L. Wu, B. Schweizer, F. Diederich, Org. Lett., 2008, 10, 3347.
7 J. D. Lewis and J. M. Moore, Dalton Trans., 2004, 1376.
8 K. Sivakumar, F. Xie and Q. Wang, Org. Lett., 2004, 6, 4603.
9 J. W. Sibert and V. Lynch, Inorg. Chem., 2007, 46, 10913; J. W. Sibert, P. B. Forshee, Inorg. Chem., 2002, 41(23), 5928.
10 D.-N. Lee, G.-J. Kim, H.-J. Kim, Tetrahedron Lett., 2009, 50, 4766.
11 E. Tamanini, S. E. J. Rigby, M. Motevalli, M. H. Todd, M. Watkinson, Chem. Eur., J. 2009, 15, 3720.
12 The FEFs were determined by division of the relative fluorescence intensities at the λₘₐₓ of the complexed and free fluoroionophores.
13 a) H. He, M. A. Mortellaro, M. J. P. Leiner, R. J. Fraatz, J. K. Tusa, J. Am. Chem. Soc., 2003, 125, 1468; b) P. Padmawar, X. Yao, O. Bloch, G. T. Manley, A. S. Verkman, Nat. Methods., 2005, 2, 825; R. D. Carpenter, A. S. Verkman, Org. Lett., 2010, 12, 1160; c) H. He, M. A. Mortellaro, M. J. P. Leiner, S. T. Young, R. J. Fraatz, J. K. Tusa, Anal. Chem., 2003, 75, 549.
14 K. Sivakumar, F. Xie, B. M. Cash, S. Long, H. N. Barnhill and Q. Wang, Org. Lett., 2004, 6, 4603-4606.
15 a) J.-S. Wu, W.-M. Liu, X.-Q. Zhuang, F. Wang and S.-T. Lee, Org. Lett., 2007, 9, 33-36.
   b) D.-N. Lee, G.-J. Kim, H.-J. Kim, Tetrahedron Lett., 2000, 50, 4766-4768.
16D. Jiménez, R. Martinez-Máñez, F. Sancenón, and E. García-Breijo, Eur. J. Inorg. Chem. 2005, 2393-2403
17 J. P. Dix and F. Vögtle, Chem. Ber. 1980, 113, 457.
18 J. D. Lewis, J. M. Moore, Dalton Trans., 2004, 1376-1385.
19 J. W. Sibert, V. Lynch, Inorg. Chem., 2007, 46, 10913-10925.
20 P. A. S. Smith and J. H. Boyer, Org. Synth., 1963, 4, 74.
21 E. Tamanini, S. E. J. Rigby, M. Motevalli, M. H. Todd, M. Watkinson, Chem. Eur. J. 2009, 15, 3720-3728.
22 M. Hirokazu; S. Furuyoshi, Y. Nakatsuji, M. Okahara. Bull. Chem. Soc. Jpn.1983, 56, 212-218.
23 R. Wortmann, C. Glania, P. Krämer, R. Matschiner, J. J. Wolff, S.

Kraft, B. Treptow, E. Barbu, D. Längle and G. Görlitz, Chem. Eur. J., 1997, 3, 1765-1773.
24 Synlett 1996, 521-522
25 Chem. Commun. 2011, 47, 4685-4687
26 Huimin Guo, Maria L. Muro-Small, Shaomin Ji, Jianzhang Zhao, Felix N. Castellano, Inorg. Chem. 2010, 49, 6802-6804
27 T. Gunnlaugsson, Paul. E. Kruger and G. M. Hussey, J. of. Org. Chem., 2005, 70(25), 10875-10878.
28 Q. Xiao, T. Brown, Tetrahedron, 2007, 63, 3483.
29 K.-C. Chang, I.-H. Su, A. Senthilvelan, W.-S. Chung, Org. Lett. 2007, 9(17), 3363-3366
30 A. Torrado, B, Imperiali, J. Org. Chem. 1996, 61, 8940-8948
31 Liu et al., Journal of Polymer Science: Part A: Polymer Chemistry 2001, 39, 1495-1504
32 Kreicberga. E. Jecs. V. Kampars. Rigas Tehniskas Universitates Zinatniskie Raksti. Serija 1: Materialzinatne un Lietiska Kimija. 2005. 10. 46-53.
33 H. He und J. K. Tusa. J. Am. Chem. Soc. 2003. 125. 1468-1469
34 D. Jimenez und E. García-Breijo. Eur. J. Inorg. Chem. 2005. 2393-2403
35 T. Gunnlaugsson und M. Nieuwenhuyzen. J. Chem. Soc. Trans. 1. 2002. 1954-1962
36 H. Guo und F. N. Castellano. Inorg. Chem. 2010. 49. 6802-6804.

1. Fluoroionophoric compound of the general formula I
Ionophore-n-Linker-Fluorophore (I)
wherein
the Ionophore is an anilino containing crown ether or cryptand with one or more anilino donor moieties as electron donors, forming a stable complex with an alkali metal ion
the π-Linker is an aromatic or heteroaromatic conjugative linking moiety, and
the Fluorophore is an electron acceptor moiety.

2. Compound, according to claim 1, wherein the ionophore is selected from the group consisting of 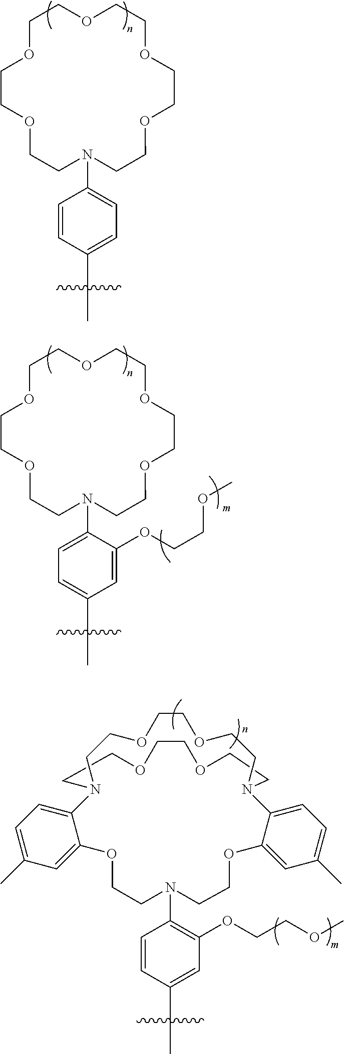
wherein
n is a number selected from 0 and 1,
m is a number selected from 0, 1, and 2, and
wherein the phenyl ring is optionally substituted with halogen, nitro, amino, hydroxyl, lower alkyl or lower alkoxy, wherein the lower alkyl or lower alkoxy are optionally substituted with halogen, nitro, amino, hydroxyl or lower alkyl or lower alkoxy, and wherein the phenyl ring may optionally be a part of a condensed aromatic system, that is optionally substituted with halogen, nitro, amino, hydroxyl, or lower alkyl or lower alkoxy or phenyl, optionally substituted with halogen, nitro, amino, hydroxyl or lower alkyl or lower alkoxy.

3. Compound, according to claim 1, wherein the π-Linker is selected from the group consisting of an aromatic or heteroaromatic moiety, wherein the aromatic moiety refers to a 6- to 14-membered monocyclic, bicyclic or tricyclic aromatic hydrocarbon ring system that is unsubstituted or optionally substituted with one or more substituents, and wherein the heteroaromatic moiety refers to an aromatic heterocyclic ring of 5 to 14 members and having at least one heteroatom selected from nitrogen, oxygen and sulfur, and containing at least 1 carbon atom, including monocyclic, bicyclic, and tricyclic ring systems, that is unsubstituted or optionally substituted with one or more substituents.

4. Compound, according to claim 3, wherein the π-Linker is selected from the group consisting of phenyl and naphthyl, that are unsubstituted or optionally substituted with one or more substituents, and triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, quinazolinyl, pyrimidyl, azepinyl, oxepinyl, naphthothiazolyl, quinoxalinyl, that are unsubstituted or optionally substituted with one or more substituents.

5. Compound, according to claim 1, wherein the π-Linker is selected from the group consisting of isomeric disubstituted 1,2,3-triazoles, preferably: 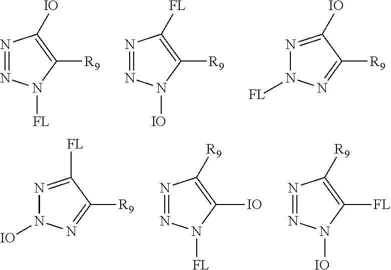
wherein
IO is the ionophore according to claim 1;
Fl is the fluorophore according to claim 1;
R₉ is selected from hydrogen, halogen, nitro, amino, hydroxyl, lower alkyl and lower alkoxy, optionally substituted with halogen, nitro, amino, hydroxyl or lower alkyl or lower alkoxy.

6. Compound, according to claim 1, wherein the π-Linker is selected from the group consisting of substituted 1,4-triazoles, namely 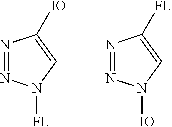
wherein IO is the ionophore and Fl is the fluorophore.

7. Compound, according to claim 1, wherein the fluorophore moiety is represented by the formula 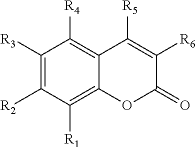
wherein
R¹, R⁴, R⁵═H, lower alkyl, CF₃, MeO, halogen, NO₂, CN,
R₂, R₃═H, NH₂, N(lower alkyl)₂, lower alkyl, optionally substituted with carboxyl or carbonyl, diethyl amino
R⁶=alkinyl, azide.

8. Compound, according to claim 1, wherein the fluorophore moiety is selected from the group consisting of 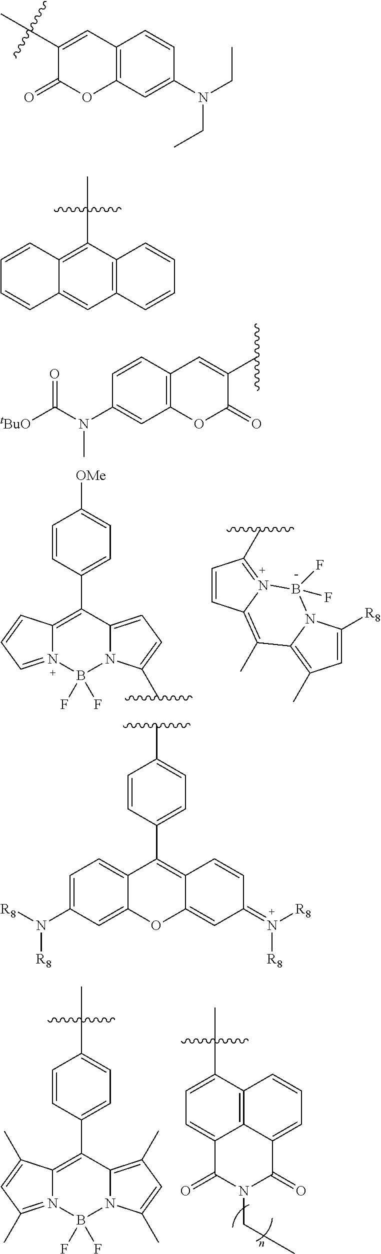
wherein
n is integer ranging from 0 to 15;
R₈ is selected from hydrogen, lower alkyl or lower alkoxy;
and which are optionally substituted with halogen, nitro, amino, hydroxyl, lower alkyl or lower alkoxy.

9. Compound, according to claim 1, namely
3-{4-[4-(1,4,7,10,13-pentaoxa-16-azacyclooctadecan-16-yl)phenyl]-1H-1,2,3-triazol-1-yl}-7-(diethylamino)-2H-chromen-2-one,
3-{1-[4-(1,4,7,10,13-pentaoxa-16-azacyclooctadecan-16-yl)phenyl]-1H-1,2,3-triazol-4-yl}-7-(diethylamino)-2H-chromen-2-one,
7-(diethylamino)-3-{1-[3-(2-methoxyethoxy)-4-(1,4,7,10,13-pentaoxa-16-azacyclooctadecan-16-yl)phenyl]-1H-1,2,3-triazol-4-yl}-2H-chromen-2-one,
7-(diethylamino)-3-{4-[3-(2-methoxyethoxy)-4-(1,4,7,10,13-pentaoxa-16-azacyclooctadecan-16-yl)phenyl]-1H-1,2,3-triazol-1-yl}-2H-chromen-2-one,
7-(diethylamino)-3-{4-3-[2-methoxyethoxy)-4{bis(4-methylbenzo[5,6,17,18](O,N,N′, O′)-]-1,7,16, -triaza-cryptand-[3,2,1]-phen-4-yl]-1H-1.2.3-triazol-1-yl}-2H-chromen-2-one,
7-(diethylamino)-3-{4-[3-(2-methoxyethoxy)-4 {bis(4-methylbenzo[5,6,17,18](O,N,N′, O′)-]-1,7, 16, -triaza-cryptand-[3,2,1]-phen-4-yl]-1H-1.2.3-triazol-1-yl}-2H-chromen-2-one,
3-{4-[4-(1,4,7,10-tetraoxa-13-azacyclopentadecan-13-yl)phenyl]-1H-1,2,3-triazol-1-yl}-7-(diethylamino)-2H-chromen-2-one,
3-{1-[4-(1,4,7,10-tetraoxa-13-azacyclopentadecan-13-yl)phenyl]-1H-1,2,3-triazol-4-yl}-7-(diethylamino)-2H-chromen-2-one,
7-(diethylamino)-3-{4-[3-methoxy-4-(1,4,7,10-tetraoxa-13-azacyclopentadec an-13-yl)phenyl]-1H-1,2,3-triazol-1-yl}-2H-chromen-2-one,
7-(diethylamino)-3-{1-[3-methoxy-4-(1,4,7,10-tetraoxa-13-azacyclopentadecan-13-yl)phenyl]-1H-1,2,3-triazol-4-yl}-2H-chromen-2-one,
7-(diethylamino)-3-{4-[3-methoxy-4 {bis(4-methylbenzo[5,6,17,18](O,N,N′,O′)-]-1, 7, 16, -triaza-cryptand-[3,2,1]-phen-4-yl]-1H-1.2.3-triazol-1yl}-2H-chromen-2-one,
7-(diethylamino)-3-{1-[3-methoxy-4 {bis(4-methylbenzo[5,6,17,18](O,N,N′,O′)-]-1,7, 16, -triaza-cryptand-[3,2,1]-phen-1-yl]-1H-1.2.3-triazol-1yl}-2H-chromen-2-one,
6-{4-[4-(1,4,7,10,13-pentaoxa-16-azacyclooctadecan-16-yl)phenyl]-1H-1,2,3-triazol-1-yl}-2-ethyl-1H-benzo[de]isoquinoline-1,3(2H)-dione,
6-{1-[4-(1,4,7,10,13-pentaoxa-16-azacyclooctadecan-16-yl)phenyl]-1H-1,2,3-triazol-4-yl}-2-ethyl-1H-benzo[de]isoquinoline-1,3(2H)-dione,
2-ethyl-6-{4-[3-(2-methoxyethoxy)-4-(1,4,7,10,13-pentaoxa-16-azacyclooctadecan-16-yl)phenyl]-1H-1,2,3-triazol-1-yl}-1H-benzo[de]isoquinoline-1,3(2H)-dione,
2-ethyl-6-{1-[3-(2-methoxyethoxy)-4-(1,4,7,10,13-pentaoxa-16-azacyclooctadecan-16-yl)phenyl]-1H-1,2,3-triazol-4-yl}-1H-benzo[de]isoquinoline-1,3(2H)-dione,
2-ethyl-6-{4-[3-(2-methoxyethoxy)-4 {bis(4-methylbenzo[5,6,17,18](O,N,N′,O′)-]-1, 7, 16, -triaza-cryptand-[3, 2, 2]-phen-1-yl]-1H-1.2.3-triazol 1-yl}-1H-benzo[de]isoquinoline-1,3(2H)-dione,
2-ethyl-6-{1-[3-(2-methoxyethoxy)-4 {bis(4-methylbenzo[5,6,17,18](O,N,N′,O′)-]-1, 7, 16, -triaza-cryptand-[3, 2, 2]-phen-4-yl]-1H-1.2.3-triazol 1-yl}-1H-benzo[de]isoquinoline-1,3(2H)-dione,
6-{4-[4-(1,4,7,10-tetraoxa-13-azacyclopentadecan-13-yl)phenyl]-1H-1,2,3-triazol-1-yl}-2-ethyl-1H-benzo[de]isoquinoline-1,3(2H)-dione,
6-{1-[4-(1,4,7,10-tetraoxa-13-azacyclopentadecan-13-yl)phenyl]-1H-1,2,3-triazol-4-yl}-2-ethyl-1H-benzo[de]isoquinoline-1,3(2H)-dione,
2-ethyl-6-{4-[3-methoxy-4-(1,4,7,10-tetraoxa-13-azacyclopentadecan-13-yl)phenyl]-1H-1,2,3-triazol-1-yl}-1H-benzo[de]isoquinoline-1,3(2H)-dione,
2-ethyl-6-{1-[3-methoxy-4-(1,4,7,10-tetraoxa-13-azacyclopentadecan-13-yl)phenyl]-1H-1,2,3-triazol-4-yl}-1H-benzo[de]isoquinoline-1,3(2H)-dione,
2-ethyl-6-{4-[3-methoxy-4{bis(4-methylbenzo[5,6,17,18](O,N,N′,O′)-]-1,7,16, -triaza-cryptand-[3,2,1]-phen-1-yl]-1H-1.2.3-triazol 1-yl}-1H-benzo[de]isoquinoline-1,3(2H)-dione,
2-ethyl-6-{1-[3-methoxy-4 {bis(4-methylbenzo[5,6,17,18](O,N,N′,O′)-]-1,7,16, -triaza-cryptand-[3,2,1]-phen-4-yl]-1H-1.2.3-triazol 1-yl}-1H-benzo[de]isoquinoline-1,3(2H)-dione,
7-{4-[4-(1,4,7,10,13-pentaoxa-16-azacyclooctadecan-16-yl)phenyl]-1H-1,2,3-triazol-1-yl}-5,5-difluoro-1,3,9,10-tetramethyl-5H-dipyrrolo [1,2-c: 1′,2′-f][1,3,2] diazaborinin-4-ium-5-uide,
7-{1-[4-(1,4,7,10,13-pentaoxa-16-azacyclooctadecan-16-yl)phenyl]-1H-1,2,3-triazol-4-yl}-5,5-difluoro-1,3,9,10-tetramethyl-5H-dipyrrolo [1,2-c:1′,2′-f][1,3,2] diazaborinin-4-ium-5-uide,
5,5-difluoro-7-{4-[3-(2-methoxyethoxy)-4-(1,4,7,10,13-pentaoxa-16-azacyclooctadecan-16-yl)phenyl]-1H-1,2,3-triazol-1-yl}-1,3,9,10-tetramethyl-5H-dipyrrolo [1,2-c:1′,2′-f][1,3,2]diazaborinin-4-ium-5-uide,
5,5-difluoro-7-{1-[3-(2-methoxyethoxy)-4-(1,4,7,10,13-pentaoxa-16-azacyclooctadecan-16-yl)phenyl]-1H-1,2,3-triazol-4-yl}-1,3,9,10-tetramethyl-5H-dipyrrolo [1,2-c: 1′,2′-f][1,3,2]diazaborinin-4-ium-5-uide,
5,5-difluoro-7 {4-[3-(2-methoxyethoxy)-4(bis(4-methylbenzo[5,6,17,18](O,N,N′,O)′)-]-1,7, 16, -triaza-cryptand-[3, 2, 2]-phen-4-yl]-1H-1.2.3-triazol-1-yl}-4-1,3,9,10-tetramethyl-5H-dipyrrolo[1,2-c:1′,2′-f][1,3,2]diazaborinin-4-ium-5-uide,
5,5-difluoro-7 {1-[3-(2-methoxyethoxy)-4(bis(4-methylbenzo[5,6,17,18](O,N,N′,O′)-]-1,7,16, -triaza-cryptand-[3, 2, 2]-phen-1-yl]-1H-1.2.3-triazol-1-yl}-4-1,3,9,10-tetramethyl-5H-dipyrrolo[1,2-c:1′,2′-f][1,3,2]diazaborinin-4-ium-5-uide,
7-{4-[4-(1,4,7,10-tetraoxa-13-azacyclopentadecan-13-yl)phenyl]-1H-1,2,3-triazol-1-yl}-5,5-difluoro-1,3,9,10-tetramethyl-5H-dipyrrolo[1,2-c:1′,2′-f][1,3,2]diazaborinin-4-ium-5-uide,
7-{1-[4-(1,4,7,10-tetraoxa-13-azacyclopentadecan-13-yl)phenyl]-1H-1,2,3-triazol-4-yl}-5,5-difluoro-1,3,9,10-tetramethyl-5H-dipyrrolo[1,2-c:1′,2′-f][1,3,2]diazaborinin-4-ium-5-uide,
5,5-difluoro-7-{4-[3-methoxy-4-(1,4,7,10-tetraoxa-13-azacyclopentadecan-13-yl)phenyl]-1H-1,2,3-triazol-1-yl}-1,3,9,10-tetramethyl-5H-dipyrrolo [1,2-c:1′,2′-f][1,3,2] diazaborinin-4-ium-5-uide,
5,5-difluoro-7-{1-[3-methoxy-4-(1,4,7,10-tetraoxa-13-azacyclopentadecan-13-yl)phenyl]-1H-1,2,3-triazol-4-yl}-1,3,9,10-tetramethyl-5H-dipyrrolo [1,2-c:1′,2′-f][1,3,2] diazaborinin-4-ium-5-uide,
5,5-difluoro-7 {4-[3-methoxy-4(bis(4-methylbenzo[5,6,17,18](O,N,N′,O′)-]-1,7,16, -triaza-cryptand-[3, 2, 1]-phen-4-yl]-1H-1.2.3-triazol-1-yl}-4-1,3,9,10-tetramethyl-5H-dipyrrolo[1,2-c:1′,2′-f][1,3,2]diazaborinin-4-ium-5-uide,
5,5-difluoro-7 {1-[3-methoxy-4(bis(4-methylbenzo[5,6,17,18](O,N,N′,O′)-]-1,7,16, -triaza-cryptand-[3, 2, 2]-phen-1-yl]-1H-1.2.3-triazol-1-yl}-4-1,3,9,10-tetramethyl-5H-dipyrrolo[1,2-c:1′,2′-f][1,3,2]diazaborinin-4-ium-5-uide,
10-(4-{4-[4-(1,4,7,10,13-pentaoxa-16-azacyclooctadecan-16-yl)phenyl]-1H-1,2,3-triazol-1-yl}phenyl)-5,5-difluoro-1,3,7,9-tetramethyl-5H-dipyrrolo[1,2-c:1′,2′-f][1,3,2]diazaborinin-4-ium-5-uide,
10-(4-{1-[4-(1,4,7,10,13-pentaoxa-16-azacyclooctadecan-16-yl)phenyl]-1H-1,2,3-triazol-4-yl}phenyl)-5,5-difluoro-1,3,7,9-tetramethyl-5H-dipyrrolo[1,2-c:1′,2′-f][1,3,2]diazaborinin-4-ium-5-uide,
5,5-difluoro-10-(4-{4-[3-(2-methoxyethoxy)-4-(1,4,7,10,13-pentaoxa-16-azacyclooctadecan-16-ylphenyl]-1H-1,2,3-triazol-1-yl)phenyl\-1,3,7,9-tetramethyl-5H-dipyrrolo[1,2-c:1′,2′-f][1,3,2]diazaborinin-4-ium-5-uide,
5,5-difluoro-10-(4-{1-[3-(2-methoxyethoxy)-4-(1,4,7,10,13-pentaoxa-16-azacyclooctadecan-16-yl)phenyl]-1H-1,2,3-triazol-4-yl}phenyl)-1,3,7,9-tetramethyl-5H-dipyrrolo[1,2-c:1′,2′-f][1,3,2]diazaborinin-4-ium-5-uide,
5,5-difluoro 10-(4-{4-[3-(2-methoxyethoxy)-4(bis(4-methylbenzo[5,6,17,18](O,N,N′, O′)-]-1,7,16, -triaza-cryptand-[3, 2, 2]-phen-4-yl]-1H-1.2.3-triazol 1-yl}1,3,7,9-tetramethyl-5H-dipyrrolo)(1,2-c:1′,2′-f)(1,3,2)diazaborinin-4-ium-5-uide,
5,5-difluoro 10-(4-{1-[3-(2-methoxyethoxy)-4(bis(4-methylbenzo[5,6,17,18](O,N,N′, O′)-]-1,7,16, -triaza-cryptand-[3, 2, 2]-phen-4-yl]-1H-1.2.3-triazol 4-yl}1,3,7,9-tetramethyl-5H-dipyrrolo)(1,2-c:1′,2′-f)(1,3,2)diazaborinin-4-ium-5-uide,
10-(4-{4-[4-(1,4,7,10-tetraoxa-13-azacyclopentade can-13-yl)phenyl]-1H-1,2,3-triazol-1-yl}phenyl)-5,5-difluoro-1,3,7,9-tetramethyl-5H-dipyrrolo[1,2-c:1′,2′-f][1,3,2]diazaborinin-4-ium-5-uide,
10-(4-{1-[4-(1,4,7,10-tetraoxa-13-azacyclopentadecan-13-yl)phenyl]-1H-1,2,3-triazol-4-yl}phenyl)-5,5-difluoro-1,3,7,9-tetramethyl-5H-dipyrrolo[1,2-c:1′,2′-f][1,3,2]diazaborinin-4-ium-5-uide,
5,5-difluoro-10-(4-{4-[3-methoxy-4-(1,4,7,10-tetraoxa-13-azacyclopentadecan-13-yl)phenyl]-1H-1,2,3-triazol-1-yl}phenyl)-1,3,7,9-tetramethyl-5H-dipyrrolo[1,2-c:1′,2′-f][1,3,2]diazaborinin-4-ium-5-uide,
5,5-difluoro-10-(4-{1-[3-methoxy-4-(1,4,7,10-tetraoxa-13-azacyclopentadecan-13-yl)phenyl]-1H-1,2,3-triazol-4-yl}phenyl)-1,3,7,9-tetramethyl-5H-dipyrrolo[1,2-c:1′,2′-f][1,3,2]diazaborinin-4-ium-5-uide,
5,5-difluoro 10-(4-[3-methoxy-4{bis(4-methylbenzo[5,6,17,18](O,N,N′,O′)-]-1,7,16,-triaza-cryptand-[3,2,1]-phen-4-yl]-1H-1.2.3-triazol-1-yl}-(1,3,7,9-tetramethyl-5H-dipyrrolo)(1,2-c:1′,2′-f)(1,3,2)diazaborinin-4-ium-5-uide, and
5,5-difluoro 10-(1-[3-methoxy-4{bis(4-methylbenzo[5,6,17,18](O,N,N′,O′)-]-1,7,16,-triaza-cryptand-[3,2,1]-phen-1-yl]-1H-1.2.3-triazol-1-yl}-(1,3,7,9-tetramethyl-5H-dipyrrolo)(1,2-c:1′,2′-f)(1,3,2)diazaborinin-4-ium-5-uide.

10. Complex, consisting of a compound according to claim 1 and an alkali metal cation.

11. Complex, according to claim 10, wherein the alkali metal cation is selected from the group consisting of Na⁺ and K⁺.

12. Method for the determination of metal cations in a sample, comprising the steps of
a) contacting the metal cations with at least one compound according to claim 1,
b) forming a complex consisting of the at least one compound and an alkali metal cation, whereupon fluorescence and/or luminescence appears or changes,
c) measuring the resulting fluorescence and/or luminescence.

13. Method, according to claim 12, wherein the metal cations are Na⁺ or K⁺, or a combination thereof.

14. Method, according to claim 12, wherein the at least one compound selectively complexes the metal cations to be determined.

15. Use of a compound according to claim 1 for the qualitative and/or quantitative determination of metal cations in a sample.
